(19) United States
(12) Patent Application Publication
(10) Pub. No.: US 2023/0352147 A1
(43) Pub. Date: Nov. 2, 2023

(54) SYSTEM AND METHOD FOR IMAGE QUALITY REVIEW OF MEDICAL IMAGES

(71) Applicant: Densitas Incorporated, Halifax (CA)

(72) Inventors: Mohamed Abdolell, Halifax (CA); Ryan Duggan, Miramichi (CA); Desmond Chung, Thornhill (CA); Dan Barzilai, Hod HaSharon (IL)

(21) Appl. No.: 18/025,798

(22) PCT Filed: Sep. 13, 2021
(86) PCT No.: PCT/CA2021/051270

§ 371 Date: Mar. 10, 2023

Related U.S. Application Data
(60) Provisional application No. 63/077,699, filed on Sep. 13, 2020.

(51) Int. Cl. G16H 30/20; G16H 40/20; G16H 30/40; G06T 7/00

(52) U.S. Cl. CPC G16H 30/20; G16H 40/20; G16H 30/40; G06T 7/0012; G06T 2207/30068

(57) ABSTRACT

Various embodiments are described herein for methods and devices for performing image quality review of medical images. In a first aspect, one of the methods include receiving an indication that an image study is being retrieved for viewing at a computing device by a user and an image study ID for the image study; retrieving image quality data that corresponds to the image study based on the image study ID, the image quality data being retrieved from a database; generating an image quality Graphical User Interface (GUI); and displaying the image quality GUI along with at least some of the image quality data at the computing device.

### CROSS-REFERENCE TO RELATED APPLICATION

This application claims the benefit of U.S. Provisional Patent Application No. 63/077,699 filed Sep. 13, 2020, and the entire contents of U.S. Provisional Patent Application No. 63/077,699 are hereby incorporated herein in its entirety.

### FIELD

Various embodiments are described herein that generally relate to systems and methods for identifying, selecting and displaying images, as well as displaying and possibly revising image quality data during review of medical images, and creating medical imaging workflows for improving the image quality review process.

### BACKGROUND

The lack of systematic processes related to clinical image quality assessment has been identified as a challenge in the medical imaging community. For example, the lack of standardized, efficient and systematic processes related to mammographic image quality review in breast cancer screening practice is a challenge and a focus of national mammography accreditation programs, particularly as it relates to conformity or non-conformity of mammograms acquired during mammographic exams with established mammography quality criteria. Various initiatives have been undertaken to identify and emphasize the need for ongoing mammography facility review of clinical image quality.

Quality Assurance (QA) processes in medical imaging are meant to ensure that reviewers (IP, LIP, QC Managers) are able to provide timely, specific feedback to the Medical Imaging Technologist (MIT) acquiring medical images on a routine basis. However, while medical imaging has progressed from film-based image acquisition to digital image acquisition, a large component of the medical image review and QA process has remained relatively unchanged.

For example, current QA processes in medical imaging are typically subject to: (a) a lack of standardization due to subjectivity and poor reproducibility in image quality review assessments between reviewers (IP, LIP, QC Managers) and MITs; (b) delayed and limited communication of QA review results; limited insights into historical QA review results; (c) limited capacity for benchmarking performance; and (d) time consuming and inefficient administrative tasks for tracking corrective actions.

Taking account of image quality during initial interpretation of medical images as well as in QA processes is important. For example, some studies have shown that image quality errors occur in as much as nearly 50% of all acquired mammograms, and patient positioning is the single most frequently occurring factor impacting image quality and accounts for nearly 80% of all image quality errors (Taplin 2002, Moieria 2005).

Therefore, current QA processes in medical imaging are prohibitively resource intensive, time consuming and subjective, which compromise the implementation of comprehensive, standardized QA processes across entire patient populations. This typically results in implementation of QA processes that are restricted in scope of image quality evaluation and in extent of patient population capture.

Furthermore, there can be missed diagnoses when images that have poor quality are not identified and reviewed. In the case of mammography, missed cancers during screening leads to delayed diagnosis and delayed treatment which is associated with poorer patient outcomes. The ability of a radiologist to detect breast cancer from mammograms depends on the image quality (Ekpo 2014) and the sensitivity of poor-quality screening mammograms is approximately 18% less than for those that are of high quality (Taplin 2002).

Poor image quality can also lead to increases in false positives, which then increases the level of anxiety for patients, as well as increases patient dose and health system costs (Guertin 2018). Whereas higher image quality has been linked to increases in cancer detection rates (Taplin 2002), as well as decreases in patient radiation dose (O'Leary 2011), interval cancer rates (Taplin 2002), and stage at cancer diagnosis (Rauscher 2013).

### SUMMARY OF VARIOUS EMBODIMENTS

In accordance with one aspect of the teachings herein, there is provided a computer-implemented method for performing image quality review of medical images, wherein the method is performed by a processor and the method comprises: receiving an indication that an image study is being retrieved for viewing at a computer device by a user and an image study ID for the image study; retrieving image quality data that corresponds to the image study based on the image study ID, the image quality data being retrieved from a database; generating an image quality Graphical User Interface (GUI); and displaying the image quality GUI along with at least some of the image quality data at the computing device.

In at least one embodiment, the image quality GUI that is displayed comprises a window that includes an image quality metric that summarizes the image quality of the image study.

In at least one embodiment, the method further comprises displaying additional measures in the image quality GUI where the additional measures are related to one or more images of the image study and comprise: breast density data, cancer risk data, a priority score or any combination thereof.

In at least one embodiment, the image quality GUI is generated and displayed when the image quality metric indicates the image study is inadequate for making a correct diagnosis by comparing the image quality criterion an image quality criterion.

In at least one embodiment, the method further includes presenting the user with a list of follow-up tasks including any combination of: (a) displaying an enhanced view of the image quality data for the image study; (b) scheduling a follow-up visual quality review of the image study; (c) sending an electronic message with the image study ID for electronic documentation and creation of a report of a review of the image study; (d) sending an electronic notification message to prioritize the image study for review; (e) sending an electronic notification message to perform a follow-up action on a patient from whom the image study was obtained; and (f) sending an electronic request message to another user to review the image study to provide a second assessment.

In at least one embodiment, the image quality GUI that is displayed comprises a subwindow having a plurality of image quality data for different images of the image study.

In at least one embodiment, the subwindow further includes images of the image study.

In at least one embodiment, the image quality data shown in the subwindow comprises names of image parameter feature scores and scores or image quality symbols for the image parameter feature scores.

In at least one embodiment, the image quality symbols comprise error symbols or pass symbols and when the user selects an edit function in the image quality GUI the method further includes displaying an opposite image quality symbol for any image quality symbols selected by the user.

In at least one embodiment, the method comprises displaying an input button in the image quality GUI for allowing the user to select that the image study is to be sent for Visual Quality Review (VQR) and the method comprises flagging the image study for VQR upon receipt of the input button being selected by the user.

In at least one embodiment, the method comprises generating a Visual Quality Review (VQR) recommendation and displaying the VQR recommendation at the computing device.

In at least one embodiment, the method comprises generating the VQR recommendation by comparing the image quality metric to an image quality threshold.

In at least one embodiment, wherein upon receiving a command from the user to send the image study to VQR, the method comprises electronically documenting that the image study is to be sent for VQR.

In at least one embodiment, wherein upon receiving a command from the user to send the image study to VQR, the method comprises updating the image quality GUI to display that the image study is to be sent for VQR.

In at least one embodiment, the method further comprises displaying a subwindow that includes breast density data in the image quality GUI.

In at least one embodiment, the method further comprises displaying a another subwindow that includes cancer risk data in the image quality GUI.

In at least one embodiment, the method further comprises displaying a an additional subwindow that includes priority score data in the image quality GUI.

In at least one embodiment, the method further comprises generating the priority score using the image quality data, the breast density data and the cancer risk data.

In at least one embodiment, the priority score is generated using a decision tree having a first level where the cancer risk data is stratified between a standard risk score and a priority risk score based on comparing a priority score value to a priority score threshold, a second level where the breast density data is stratified between high density or low density based on comparing a breast density value to a breast density threshold and a third level where the image quality data is stratified between high quality and poor quality based on comparing an overall image quality value for the image study to an image quality threshold.

In another aspect, in accordance with the teachings herein, there is provided at least one embodiment of an electronic device for providing image quality review of medical images in a medical imaging system, wherein the electronic device comprises: a memory unit that includes software instructions for visualizing image quality data; a network unit for communicating with other devices and software programs in the medical imaging system; and a processor unit in communication with the memory unit and the network unit, the processor unit having at least one processor that is configured to: receive an indication that an image study is being retrieved for viewing at a computing device by a user and an image study ID for the image study where the computing device is electronically connected to the medical imaging system; retrieve image quality data that corresponds to the image study based on the image study ID, the image quality data being retrieved from a database; generate an image quality Graphical User Interface (GUI); and display the image quality GUI along with at least some of the image quality data at the computing device.

In at least one embodiment, the at least one processor unit is further configured to perform the above-noted method.

In another aspect, in accordance with the teachings herein there is provided at least one embodiment of a computer readable medium comprising software instructions, which when executed by an electronic device, configure the electronic device to perform the above-noted method.

In another aspect, in accordance with the teachings herein, there is provided at least one embodiment of a computer-implemented method for automatically identifying image studies for image quality review using a medical imaging system, wherein the method is performed by a processor and the method comprises: receiving an indication that a new image study has been acquired and an image study ID for the new image study; obtaining image quality data for images in the new image study; determining when the image quality data meets Visual Quality Review (VQR) criteria; and updating a VQR worklist file to include the image study ID when the image quality data meets the VQR criteria.

In at least one embodiment, the method comprises: generating and displaying an image quality search Graphical User Interface (GUI) that provides input fields to allow a user to enter the VQR criteria; receiving the VQR criteria from the user; and saving the received VQR criteria.

In at least one embodiment, the method comprises displaying an input option for allowing the user to select one or more of views or a portion of the images in the new image study the VQR criteria are applied to.

In at least one embodiment, the method comprises (a) displaying input options to the user to allow the user to specify the VQR criteria that are applied to certain images of the new image study, (b) receiving at least one VQR criterion from the user or a user defined combination, via at least one logical operator, of at least two VQR criteria where each VQR criteria, where each VQR criteria involves an image parameter feature that is selected by the user, a comparison operator that is selected by the user and a threshold value that is selected by the user; and (c) storing these user selections for the VQR criteria.

In at least one embodiment, the method comprises keeping track when a number of image studies that were acquired by a given Medical Imaging Technologist (MIT) meet the VQR criteria over a certain time period, VQR review for the given MIT is electronically noted.

In at least one embodiment, the method comprises keeping track when a number of image studies that were acquired by a given MIT are flagged based on the VQR criteria and determining whether an overall image study quality, across all of the flagged image studies, for the MIT drops below a typical image quality level for the MIT over a certain time period.

In another aspect, in accordance with the teachings herein, there is provided at least one embodiment of a method for randomly generating a list of image studies for Visual Quality Review (VQR) using a medical imaging system, wherein the method is performed by a processor and the method comprises: displaying a first section for a random search Graphical User Interface (GUI), where the first section includes at least one first input option to allow a user to select one or more initial search criteria; displaying a second section for the random search GUI where the second section includes at least one second input option for allowing the user to select one or more stratifying factors; displaying a third section for the random search GUI where the third section includes a third input option to allow the user to specify a desired number of images studies for VQR for a given pairing of a Medical Imaging Technologist (MIT) and an Interpreting Physician (IP) in a random sample where the MIT is a person who acquires the image studies and the IP is a person who reviews image quality of the image studies; receiving the user selections for the input options; generating a random list of image studies for VQR using the user selections; and storing the random list of the image studies for VQR.

In at least one embodiment, the method further comprises displaying a recommended number of image studies for VQR for the given pairing of the MIT and the IP in the random sample.

In at least one embodiment, the method further comprises displaying a number of image studies for VQR based on the user selections.

In at least one embodiment, the at least one first input option includes an institution, a department for the institution, a date range, one or more MIT selections and/or one or more IP selections.

In at least one embodiment, the method further comprises displaying a potential number of image studies for VQR based on the user selections to the at least one first input option.

In at least one embodiment, the one or more stratifying factors include scanner model, breast density value and/or image quality metric score.

In another aspect, in accordance with the teachings herein, there is provided at least one method for electronically performing Visual Quality Review (VQR) on at least one image study that is acquired by a Medical Imaging Technologist (MIT) using a medical imaging system, wherein the method is performed by a processor and the method comprises: sending an electronic request to a reviewer to perform VQR on a first image study; retrieving image quality data that corresponds to the image study based on the image study ID, the image quality data being retrieved from a database; generating one or more image quality Graphical User Interfaces (GUIs) that include at least a portion of the image quality data; displaying the one or more image quality GUIs; and receiving and storing image quality feedback data from the reviewer.

In at least one embodiment, the one or more GUIs include a summary of the image quality data including image quality categories, an index of possible scores for the image quality categories and a score value for the image quality categories.

In at least one embodiment, the one or more GUIs include an image quality category; optionally a list of possible scores for the image quality category and a score value the image quality category; and a list of image quality parameter features for the image quality category.

In at least one embodiment, the one or more GUIs include input fields for the list of image quality parameter features of the image quality category for one or more images of the first image study.

In at least one embodiment, the image quality categories comprise positioning, compression, exam ID, artifacts, exposure, contrast, sharpness, noise or any combination thereof.

In at least one embodiment, the method further comprises generating an additional GUI to allow the reviewer to select whether to initiate a review follow-up or to indicate that the VQR is complete, and no further action is needed and receiving a selection from the reviewer.

In at least one embodiment, the method further comprises generating an automated recommendation on whether the first image study had an overall level of image quality that is acceptable for interpreting the image study to provide an accurate diagnosis and displaying the automated recommendation to the reviewer.

In at least one embodiment, the method further comprises generating the automated recommendation by generating a VQR score, comparing the VQR score to a VQR threshold; and determining whether initiation of a review follow-up is suitable based on the comparison.

In at least one embodiment, the VQR score is generated based on a weighted sum of image parameter feature scores across image quality categories from the image quality data for the first image study.

In at least one embodiment, the VQR threshold is a predefined value, a prognostic index based on a weighted score from regression coefficients or is determined using an algorithm where the VQR threshold is selected to identify patients who are likely to have a recall for further medical imaging to be performed on them due to inadequate image quality from at least one image study performed on the patient.

In at least one embodiment, the algorithm involves applying a statistical model to patient data to generate a classifier that employs a technical recall and no technical recall classes, where the statistical model uses a classification tree, logistic regression or Maximum Likelihood.

In another aspect, in accordance with the teachings herein, there is provided at least one embodiment of a method for electronically reviewing image quality of image studies acquired by a Medical Imaging Technologist (MIT) using a medical imaging system, wherein the method is performed by a processor and the method comprises: receiving an electronic request from a reviewer to perform a review on the MIT for a selected time period; retrieving image quality data that corresponds to image quality data for at least one image study performed by the MIT and retrieving MIT performance data for the MIT for the selected time period, the image quality data and performance data being retrieved from at least one database; generating a MIT review Graphical User Interface (GUI) that includes at least a portion of the image quality data and the MIT performance data; and displaying the MIT review GUI on a computing device used by the reviewer.

In at least one embodiment, the MIT review GUI includes images from an image study that the MIT performance is being reviewed for, and an image quality summary section that includes Visual Quality Review (VQR) results for one or more image quality categories.

In at least one embodiment, the MIT review GUI includes an assessment of whether the image study was acceptable for interpretation.

In at least one embodiment, the MIT review GUI further includes one or more image quality parameters for a selected image quality category for a selected time period.

In at least one embodiment, for a given image quality parameter, a text field is shown to display a name of the image quality parameter, a percentage indicator is shown to indicate a percentage of all of images that were acquired by the MIT during the selected time period that satisfy a particular operating point for the image quality parameter, and an identifier is shown to indicate a number of images that were unacceptable for the image quality parameter over the total number of images that were assessed.

In at least one embodiment, the MIT review GUI includes a progress chart for one or more image quality parameters to show a change in MIT performance for the one or more image quality parameters.

In at least one embodiment, the change in MIT performance is displayed with an extent to visually show if there is a big or small change in the MIT performance and a directionality to show if there is an improvement or worsening of the MIT performance.

In at least one embodiment, the method further comprises receiving a selected image quality parameter; generating a second performance graph for the MIT performance for the selected image quality parameter; displaying the MIT performance for the selected image quality parameter and displaying performance benchmark data for the selected image quality parameter in the MIT review GUI.

In at least one embodiment, the method further comprises displaying performance of the MIT for a subsequent VQR in the MIT review GUI by showing images from another image study that was reviewed for the subsequent VQR along with includes the VQR results for one or more image quality categories for the subsequent VQR.

In at least one embodiment, the MIT review GUI includes a subsequent review input option to allow the reviewer to add another subsequent VQR for review of the MIT.

In at least one embodiment, the method further comprises displaying a corrective actions section showing corrective actions that have been recommended to the MIT and additional data on whether the recommended corrective actions were taken for the MIT.

In at least one embodiment, the method comprises providing a corrective action input option to allow the reviewer to add input details for at least one new corrective action for the MIT to perform and saving any added new corrective actions.

In at least one embodiment, the method comprises providing comment text box to allow the reviewer to add comments related to progress or challenges of the MIT; or comments related to how any of the corrective actions were received and/or performed by the MIT; and saving any comments entered by the reviewer.

In at least one embodiment, the method comprises generating a review report GUI that is accessible by the MIT to provide the MIT with any recommended corrective actions to improve image quality performance; recording interaction of the MIT with the review report GUI and recording behaviour by the MIT in performing any of the recommended corrective actions.

In another aspect, in accordance with the teachings herein, there is provided an electronic device for electronically reviewing image quality of image studies acquired by a Medical Imaging Technologist (MIT) using a medical imaging system, wherein the electronic device comprises: a memory unit that includes software instructions for visualizing image quality data; a network unit for communicating with other devices and software programs in the medical imaging system; and a processor unit in communication with the memory unit and the network unit, the processor unit having at least one processor that is configured to perform any of the methods described in accordance with the teachings herein.

In another aspect, in accordance with the teachings herein, there is provided a computer readable medium comprising software instructions, which when executed by an electronic device, configure the electronic device to perform any of the methods described in accordance with the teachings herein.

Other features and advantages of the present application will become apparent from the following detailed description taken together with the accompanying drawings. It should be understood, however, that the detailed description and the specific examples, while indicating preferred embodiments of the application, are given by way of illustration only, since changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows a block diagram of an example embodiment of a medical imaging system that incorporates image quality at various stages of medical image review and quality assurance for a medical institution.

FIG. 2 shows a block diagram for an example embodiment of a medical image quality analysis server that can be used with the system of FIG. 1.

FIG. 3A shows a flow chart of an example embodiment of a method for notifying an Interpreting Physician when the image quality assessment for an image study that is being reviewed has inadequate image quality.

FIG. 3B shows an example embodiment of a Graphical User Interface (GUI) that can be used to provide the image quality notification for the method of FIG. 3A.

FIG. 4A shows a flow chart of an example embodiment of a method for providing image quality data when an Interpreting Physician is reviewing a selected image study.

FIG. 4B shows an example embodiment of a GUI for providing detailed image quality data along with a recommendation for Visual Quality Review (VQR).

FIG. 4C shows an example embodiment of a technique for determining a priority score that incorporates cancer risk prediction, breast density and image quality data and can be used to make a recommendation for VQR for the selected image study.

FIG. 4D shows an example of a pop-up window for the GUI of FIG. 4C where the pop-up window provides a rationale for recommending a VQR.

FIG. 4E shows an example embodiment of the GUI of FIG. 4D that is updated to show the reviewer's decision for performing a VQR.

FIG. 4F shows an example embodiment of a GUI for providing detailed image quality data while not presenting a recommendation for VQR but providing an input button allowing the reviewer to select VQR.

FIG. 5A shows a flow chart of an example embodiment of a method that uses randomization for automatically identifying image studies for image quality review.

FIG. 5B shows an example embodiment of a GUI that can be used with the method of FIG. 5A to set criteria for automatically identifying image studies for image quality review.

FIG. 6A shows a flow chart of an example embodiment of a method for randomly identifying image studies for image quality review.

FIG. 6B shows an example embodiment of a GUI that can be used with the method of FIG. 6A to set criteria for randomly identifying image studies for image quality review.

FIG. 7A shows an example embodiment of a method for investigating and recording operator performance during an initial or subsequent VQR of an image study.

FIG. 7B shows two portions of an example embodiment of a GUI for showing image quality scores indicating operator performance for several image acquisition errors.

FIG. 7C shows an example embodiment for determining a VQR score based on the image quality scores of FIG. 7B.

FIG. 7D shows an example embodiment of a GUI for allowing a reviewer to indicate whether the mammogram assessed with the errors shown in FIGS. 7G-7N is acceptable for interpretation by an interpreting physician.

FIG. 7E shows an example embodiment of a GUI for recommending whether to perform an operator review follow-up based on the VQR score following completed reviewer assessments shown in FIGS. 7B, 7C and 7D.

FIG. 7F shows the example embodiment of the GUI of FIG. 7E along with a pop-up window to recommend operator review follow-up.

FIGS. 7G-7N shows an example embodiment of GUIs for allowing a reviewer to enter further feedback for operator performance for errors (and optionally for future VQR modelling) during image acquisition of a mammogram for positioning, compression, exam ID, artifacts, exposure, contrast, sharpness, and noise.

FIG. 8A shows a flow chart for an example embodiment of a method for displaying MIT-specific quality performance metrics along with benchmarks, results from subsequent VQR(s), and documented corrective actions.

FIGS. 8B-8F show various portions of an example embodiment of a GUI for displaying the MIT-specific quality performance metrics along with benchmarks, results from subsequent VQR(s), and documented corrective actions.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have an electrical or electronic communication connotation. For example, as used herein, the terms coupled or coupling can indicate that two elements or devices can be directly connected to one another or connected to one another through one or more intermediate elements or devices via an electrical signal, an electrical connection, or a communication pathway depending on the particular context.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

In addition, it should be noted that the phrases “at least one of X and Y” or “X, Y or a combination thereof” is intended to mean X, Y or X and Y.

It should also be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term that it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

The embodiments of the systems and methods described herein are implemented using a combination of hardware and software. The embodiments described herein may be implemented with computer programs executing on programmable devices, each programmable device including at least one processor, a data storage system (including volatile memory or non-volatile memory or other data storage elements or a combination thereof), and at least one communication interface. For example, and without limitation, the programmable devices may be a server, a network appliance, an embedded device, a personal computer, a laptop, a personal data assistant, a smart-phone device, a tablet computer, or any other computing device capable of being configured to carry out the methods described herein where these devices may communicate using wired or wireless communications protocols as appropriate.

Program code may be applied to input data to perform the functions described herein and to generate output data. The output data may be displayed to a user via one or more output devices and/or electronically communicated to another devices. Each program may be implemented in a high-level procedural or object-oriented programming and/or scripting language, or both, to communicate with a computer system. The program code may be written in C++, C#, JavaScript, Python, MATLAB, or any other suitable programming language and may comprise modules or classes, as is known to those skilled in object-oriented programming. In either case, the language may be a compiled or interpreted language. However, the programs may be implemented in assembly or machine language, if desired. In any case, the language may be a compiled or an interpreted language. Each such computer program may be stored on a non-transitory computer-readable storage medium (e.g., ROM, magnetic disk, optical disc) that is readable by a general or special purpose computing device, for configuring and operating the computing device when the storage media or device is read by the computing device to perform one or more of the procedures in accordance with the teachings herein.

Furthermore, the functionality of the system, processes and methods of the described embodiments are capable of being distributed in one or more computer program products comprising a computer readable medium that bears computer usable instructions for one or more processors. The medium may be provided in various forms, including non-transitory forms such as, but not limited to, one or more diskettes, compact disks, tapes, chips, and magnetic and electronic storage media as well as transitory forms such as, but not limited to, wireline transmissions, satellite transmissions, internet transmission or downloads, digital and analog signals, and the like. The computer useable instructions may also be in various forms, including compiled and non-compiled code.

### Definitions

Throughout this specification and the appended claims various words and phrases are defined. Once defined, the use of these terms herein shall bear the defined following meanings.

The use of any of the terms “Graphical User Interface”, “GUI”, “window” or “pop-up message” in conjunction with describing the operation of any computing device, system or method described herein is meant to be understood as describing a user interface that is generated using software and shown on a display, monitor or screen for allowing a user to provide control inputs to control one or more of the methods described herein as well as to view image quality data, metrics or messages.

The terms “Quality Assurance” or “QA” mean the maintenance of a desired level of quality in a service or product, especially by means of attention to one or more stages of the process of performing image acquisition, delivery of resulting images and quality data regarding the images and/or production of reports on the performance of image acquisition and the resulting image quality.

The terms “Picture Archiving Communications System” or “PACS” mean a system for storing and allowing facile access to high-quality radiologic images and accompanying image meta-data. Such a system may be based on the DICOM (Digital Imaging and Communications in Medicine) standard, and may provide storage, access and manipulation services through network connections.

The terms “Radiology Information System” or “RIS” mean a software system for managing medical imaging operations and associated data, which may include imaging requests, status tracking of the imaging requests, and storage of reports from interpretations generated by the Reporting Services Software. The system may be accessed and manipulated through network connections.

The term “Reporting Services Software” means software that is used for reporting the interpretation of medical imaging studies. The report may be in the form of free-text (dictated or typed) or discrete data elements.

The term “Worklist Services Software” means software that manages a radiologist's reading workflow by presenting and organizing each radiologist's reading tasks. Some solutions may provide automatic organization of the reading tasks.

The term Medical Image Quality Assurance (MIQA) means networked software that is used for medical image-based quality assurance. In various embodiments described herein, MIQA can provide one or more of: (a) on-demand standardization and reproducibility of image quality reviews; (b) synchronous and non-synchronous feedback to MITs on positioning technique and performance; (c) efficient and effective processes for identifying poor quality images, and implementing, communicating and tracking of corrective actions triggered by poor quality images; (d) reduction of missed, delayed and limited implementation of corrective actions; (e) efficient and feasible compilation of comprehensive QA review results for imaging facility inspections/audits; and (f) storage and analytics on associated data.

The term “Context Sharing” means notification to MIQA from networked software (e.g., a PACS/RIS/Workstation/Worklist) of a medical imaging study that is currently under review at a computing device having a display by an Interpreting Physician or other reviewer that prompts the display of related data and information by MIQA software on the display of the computing device.

The term “Medical Images” means digital images created of various parts of the human body, or of material samples taken from the human body, for diagnostic or treatment purposes created with various techniques and processes such as, but not limited to, optical, X-ray, ultrasound, magnetic resonance, computed tomography (CT), or nuclear medicine such as positron-emission tomography (PET), for example.

The term “MQSA EQUIP” means the United States Food and Drug Administration Mammography Quality Standards Act, Enhancing Quality Using the Inspection Program.

The terms “Medical Imaging Technologist” or “MIT” mean an individual who is trained in the use of medical imaging equipment and the positioning of patients for acquiring medical images using imaging hardware when performing medical imaging examination.

The terms “Lead Interpreting Physician” or “LIP” mean the Interpreting Physician who is assigned the general responsibility for ensuring that a medical facility's quality assurance program meets all of the requirements.

The term “Medical Physicist” means a physicist trained to apply physics concepts, theory and methods to medicine and healthcare.

The term “QC Manager” means an individual who is responsible for those quality assurance responsibilities not assigned to the Lead Interpreting Physician or to the Medical Physicist.

The term “Reviewer” can be used to refer to an Interpreting Physician, a Lead Interpreting Physician, or a QC Manager.

The terms “Visual Quality Review” or “VQR” mean a visual evaluation of a particular set of medical images, performed by a Reviewer, that assesses various aspects of clinical image quality (e.g., positioning, exposure, contrast).

### DESCRIPTION OF VARIOUS EMBODIMENTS

The current practice for medical image quality assessment is a resource-intensive process performed by reviewers that is time-consuming and non-standardized. It is estimated to take an experienced reviewer over 10 minutes to perform a comprehensive image quality assessment. The resources required to perform these assessments across the board are simply not available, nor is it economically viable. For example, for mammographic imaging, millions of mammograms are performed annually, and comprehensive image quality reviews are only conducted on a very small sample of image studies by experienced reviewers, and because of the considerable associated time requirements comprehensive image quality reviews are not performed for the full population of women who have had a mammogram.

Accordingly, two challenges in clinical image quality review include: (1) evaluating medical images transactionally and comprehensively on-demand as they are acquired, and (2) collectively and concurrently evaluating large samples of medical images from entire patient populations for resource allocation, workload assignment, worklist filtering and prioritization, continuous quality improvement, service delivery, training, and continuing education.

Referring to the challenge of evaluating medical images comprehensively on-demand, there are several technical challenges including:
(1) there are no mechanisms for providing ongoing feedback on image quality;
(2) there are no systems in place that include mechanisms for documenting any needed corrective action nor the effectiveness of any corrective action taken;
(3) there are no systems in place for regular and comprehensive reviews of image quality attributes of a random sample of mammograms performed by each active MIT and accepted for interpretation by each active IP; (4) there is no documentation of clinical image quality review since the last inspection; and
(5) there is no system in place for MIT oversight, including review of the performance of active MITs and determining whether appropriate corrective actions were performed when needed.

Another issue is that mammography facility accreditation audits are designed to evaluate if a clinic can demonstrate their knowledge of what a properly acquired medical image should look like. Such audits are intended to evaluate competency, not performance. Despite the fact that clinics provide self-selected medical images that demonstrate adequate clinical image quality, some accreditation audits have yielded non-conformity rates as high as 10%. However, audits using random samples of digital images provide an estimate of the actual magnitude of image quality non-conformity rates (e.g., poor image quality rates) at the population level, and have yielded as high as 50% non-conformity rates. Prohibitively high costs and shortage of resources are a challenge facing healthcare systems globally, and it is not feasible for reviewers to perform comprehensive mammography image quality reviews on every single digital mammogram that is acquired, nor to implement continual quality control processes for on-demand comprehensive population-wide mammography image quality reviews.

The inventors have found that a technical solution to the various challenges listed above includes standardization through digitalization and enhanced visualization of QA processes for digital image acquisition at imaging facilities in accordance with at least one of the embodiments that are part of the teachings herein. This makes it possible to evaluate image quality on every acquired image in a patient population for comprehensive population-wide QA with a focus on reducing missed diagnoses, unnecessary imaging and poor prognosis. However, the inventors have also found that the technical solution to the challenges described herein also involves the timely and on-demand access to standardized image quality data during different stages of the medical image interpretation, review and quality assurance process.

In another aspect, in accordance with the teachings herein, at least one example embodiment is described herein to provide a technical solution identified for improved quality assurance processes which involves providing the ability to identify a random sample of mammograms for image quality review.

In another aspect, in accordance with the teachings herein, at least one example embodiment is described herein to provide a technical solution for incorporating the visualization of image quality data for performing comprehensive image quality reviews.

In another aspect, in accordance with the teachings herein, at least one example embodiment is described herein, to facilitate and improve efficiency and accuracy for digitalized benchmarking and monitoring performance of MITs across one or more medical institutions.

These various embodiments, whether performed separately or combined together, make it feasible to perform comprehensive mammography quality audits, and rapidly identify and resolve root-causes of non-conformities by using image quality data in a standardized manner. These embodiments include using an electronic database containing comprehensive image quality data on mammograms acquired from a population of women that can be accessed on-demand for analysis and workflow creation.

Although the various example embodiments described herein are with respect to mammographic images, it should be understood that the various teachings herein can be applied to retrieving and/or assessing image quality for digital medical images of other body parts of a patient's anatomy where the patient may be a person or an animal. For example, the teachings herein may be applied to chest images, cardiac images, bone images (e.g., including images of the hand, hip, knee, and/or spine), musculoskeletal (MSK) images, neurological images, oncology images, pediatric images, kidney images, orthopedic images and gastrointestinal images, for example, that may be obtained using a variety of imaging modalities such as X-ray, CT and MRI. For example, the medical system may be applied to (a) digital x-ray images of the chest, ribs, abdomen, cervical spine, thoracic spine, lumbar spine, sacrum, coccyx, pelvis, hip, femur, knee, tibia/fibula, ankle, foot, finger, hand, forearm, elbow, humerus, shoulder, sternum, AC joints, SC joints, mandible, facial bones, and/or skull; (b) digital CT images of the head, neck, chest, abdomen, pelvis, breast and/or extremities and (c) digital MRI images of the head, neck, chest, abdomen, pelvis, breast and/or extremities. Therefore, the digital mammographic images described herein are just one example of a medical images that can be assessed for quality using the teachings described herein.

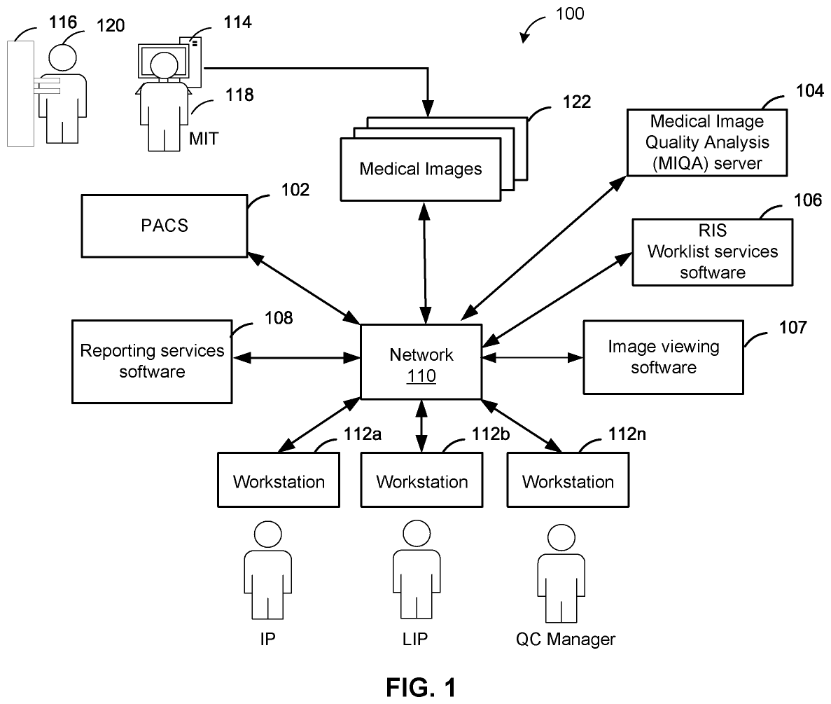

Referring now to FIG. 1, shown therein is an example embodiment of a medical imaging system 100 that incorporates image quality at various stages of medical image review as well as quality improvement and assurance for a medical institution using computing devices. The medical imaging system comprises a PACS 102, a MIQA server 104, RIS and worklist services software 106, image viewing software 107 and reporting services software 108 that communicate with one another via a network 110. The medical imaging system 100 is accessible by various computer workstations 112a to 112n that are used by different medical professionals for interpreting some of the medical images 122 and/or performing a quality review of some of the medical images 122. The RIS and worklist software 106 as well as the reporting software 108 may be executed using one or more servers. In this example, the workstations 112a to 112n may be used by IPs, LIPs and QC managers.

In other embodiments, the worklist services software 106 and the reporting services software 108 may be provided by a single software application, or by multiple collaborating software applications. Furthermore, the worklist services software 106 and the reporting services software 108 can be executed using separate unique servers, or they can both be executed on a single common server.

In some embodiments, the RIS and worklist services software 106, the image viewing software 107 and the reporting service software 108 may be provided by a single unified implementation or with other software systems. For example, the PACS 102 may also implement the RIS and worklist services software 106 and the image viewing software 107 in some embodiments

In another alternative embodiment, the PACS 102, the reporting services software 108, as well as the RIS and worklist services software 106 may all be executed using one server. In some cases, there may also be a single combined RIS/PACS solution that provides the functionality of the PACS 102, and the RIS and worklist services software 106.

In yet another example embodiment, the MIQA server 104 may be implemented on a virtual machine, so that a single set of underlying hardware resources (i.e., a single computer) may be shared by a number of ‘virtual machine’ instances. The operating system and software services running within each virtual machine, behave as if the operating system is executing on dedicated computing hardware while, in fact, the hardware is being shared across multiple virtual machines using a ‘hypervisor’, which is software that is used to host multiple virtual machines on a single piece of hardware. An example of a hypervisor is VMware ESXi.

In another embodiment, any of the PACS 102, the MIQA server 104, as well as the RIS and worklist services software 106 may be implemented using separate virtual machines which can operate on a single hardware server, but each virtual machine can be considered to be a complete and an independent computer system.

The medical images 122 are obtained by a MIT 118 who uses a computer system 114 to operate a medical imaging machine 116 to obtain medical images, which in this example embodiment is a mammographic machine used to obtain mammographic images, from a patient 120. The mammographic machine uses a parallel plate compression means to even the thickness and spread out a patient's breast tissue, delivers X-rays from an X-ray source to the compressed breast tissue, and then records the image with a detector. The medical imaging machine 116 and the computer system 114 may be co-located at a physical location with the medical imaging system 100. The computer system 114 may be a desktop computer, mobile device, laptop computer, or an embedded system associated with the medical imaging system itself. While there is only one depiction of a MIT 118, a computer system 114, and a medical imaging machine 116 in FIG. 1, it should be understood that there are typically many MIT 118 who may be working with different computer systems and medical imaging machines to obtain medical images 122 which are then sent over the network 110 for storage on the PACS 102. These medical images 122 and may be in a digital format. Alternatively, the medical imaging machine 116 may record the mammographic image on film, and the film image may then be separately digitized and transmitted to the PACS 102. In either case, the medical images that are assessed to determine quality metrics and reviewed for QA are in a digital form.

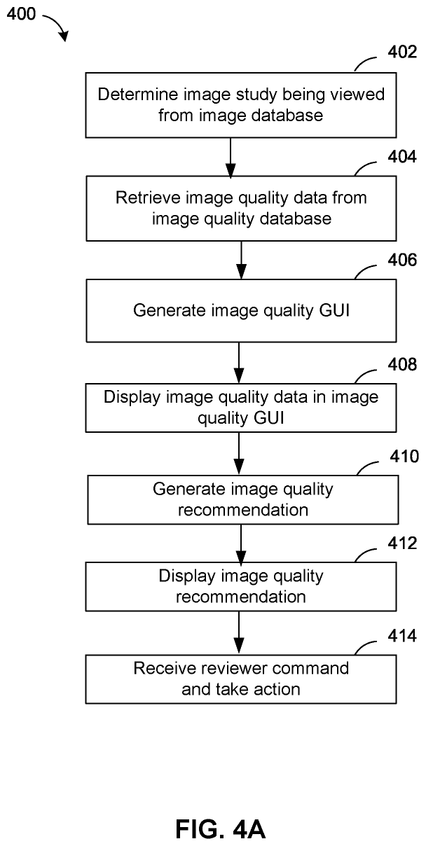

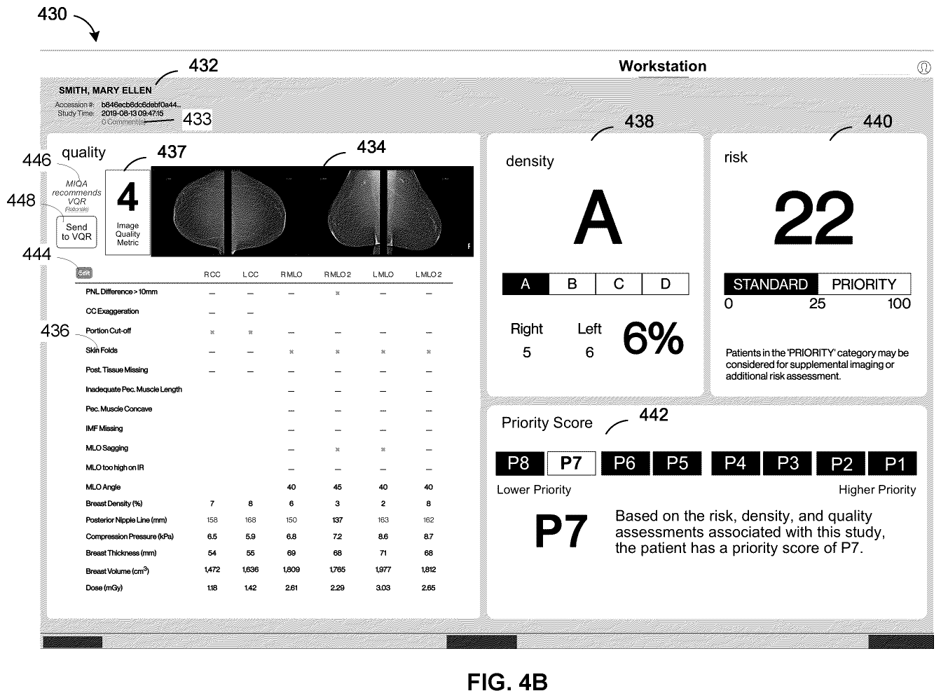

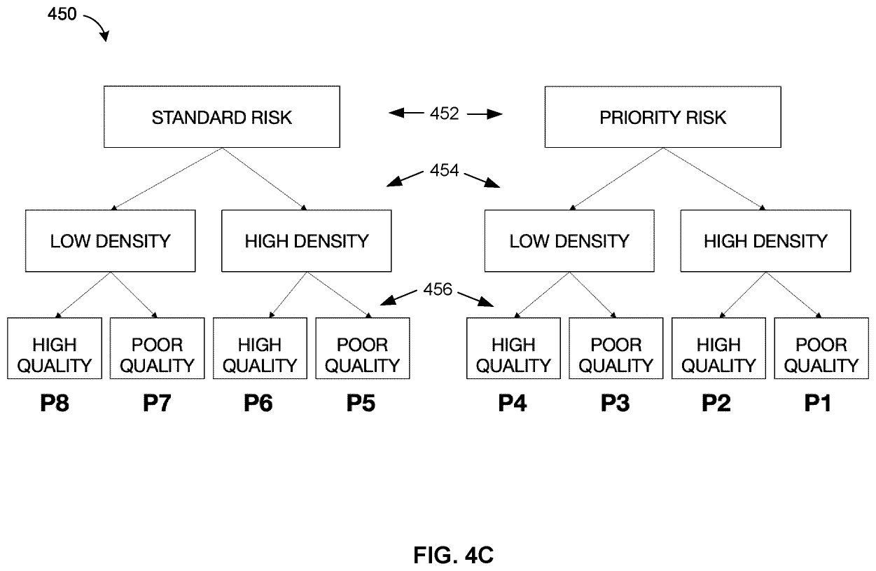

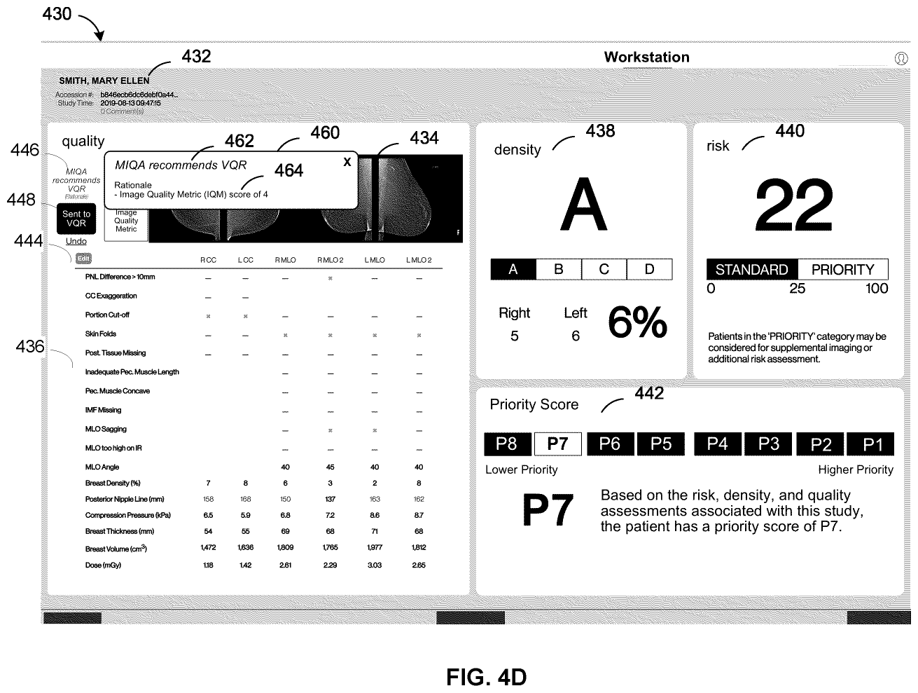

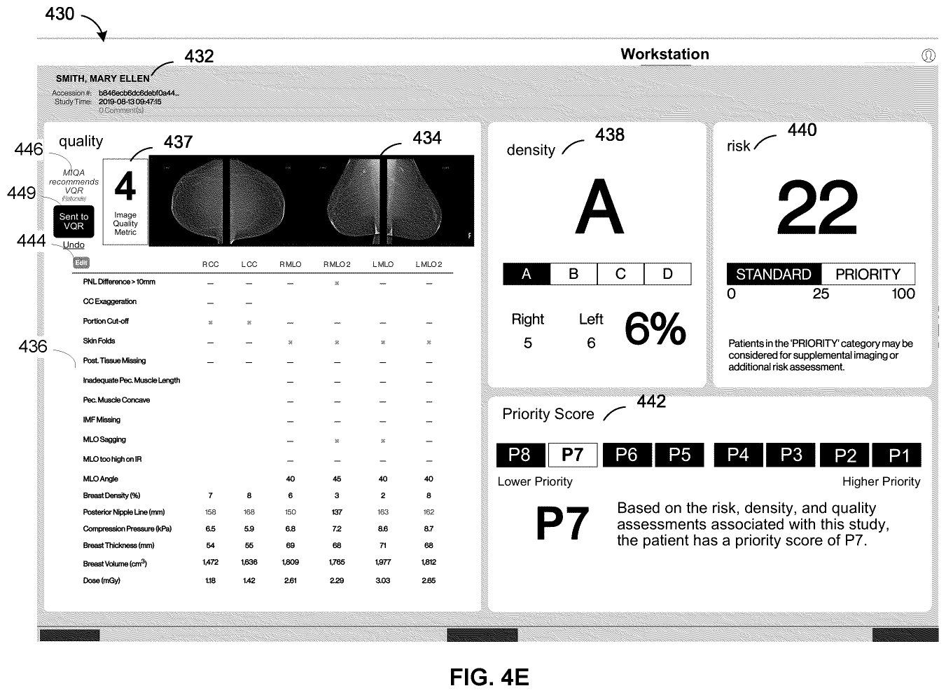

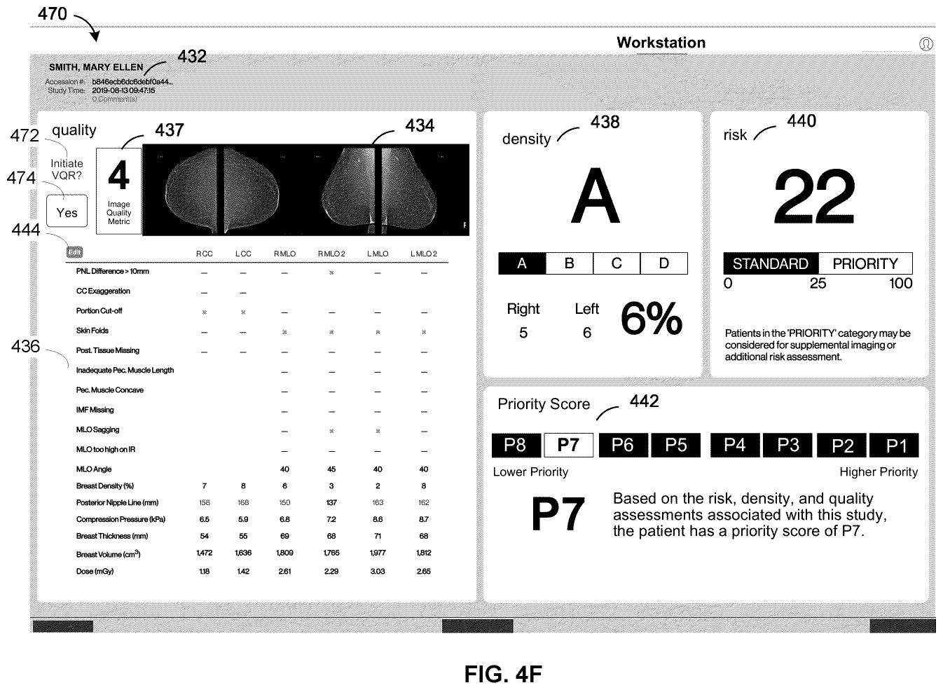

A given MIT 118 obtains a collection of different medical images from a given patient 120 during a patient examination session and the collection of medical images 122 can be referred to as an image study. The different images in an image study may include images that are taken of a region of interest or body part at different angles, or images that are taken of different positions of a portion of the target body part or a region of interest. For example, for mammography, a given image study typically includes images taken from certain views including a Right Craniocaudal (RCC) view, a Left Craniocaudal (LCC) view, a Right Mediolateral Oblique (RMLO) view and a Left Mediolateral Oblique (LMLO) view. There may be multiple images for each of the CC view and the MLO view, such as one image for each breast in each view. An example of images obtained from these views is shown near the top left corner of FIG. 4B.

The medical imaging system 100 may associate a plurality of metadata with the image data for the medical images 122. The image data may be in any format known in the art, such as JPEG, TIFF, or PNG. The image data may also be in any of the standard DICOM pixel data formats (which might use uncompressed, JPEG, JPEGLossless or other formats), packaged within a DICOM file object. The metadata may include acquisition settings data, patient data (such as patient ID, patient sex and patient age), image machine data, institution data, and MIT data. The metadata and the image data may be combined according to a standardized data format such as the DICOM data format. This metadata is further described in PCT application publication WO 2020/102914 which was published on May 28, 2020 and is hereby incorporated by reference in its entirety.

The PACS 102 is in network communication with other components of the medical image system 100 including the MIT computer system 114, the MIQA server 104, the reporting services software 108, the RIS and worklist services software 106 and the various workstations 112a to 112n. The PACS 102 receives the medical images 122 and the plurality of corresponding image metadata and stores them in a database from the medical imaging system 100.

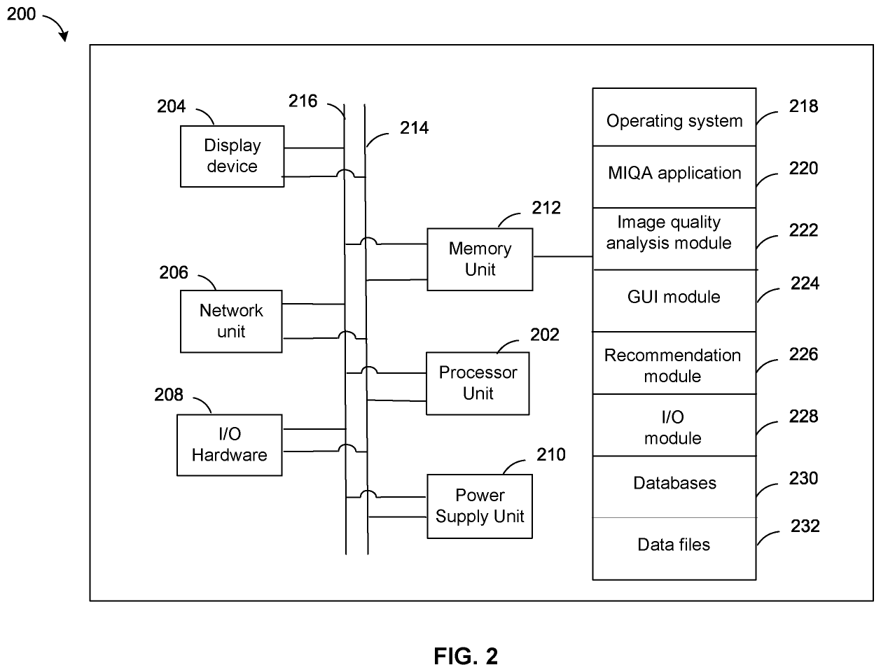

The MIQA server 104 executes various programs for analyzing the medical images 122 for one or more image studies and determining the corresponding image quality data. The image quality data may include one or more of IQPs, IQPFs, IQPSs, IQPI, SQPs, SQPSs and SQPIs, as described herein, with respect to the description of the MIQA server 104 with respect to FIG. 2. The image quality data may be stored on memory at the MIQA server 104 or on a separate data store. The image quality data may then be used to assist with interpretation of one or more medical images of a corresponding image study at the time of interpretation or during QA processes. In other alternative embodiments, other types of image quality data may be used to provide for enhanced visualization and assessment of medical images according to the various methods described herein.

The MIQA server 104 may retrieve the medical images 122 for one or more image studies from the PACS 102 just after the time of acquisition by the MIT 118 to give real-time feedback on image quality to the MIT 118 or at a later time to generate the image quality data for each image study. The MIQA server 104 can communicate with the PACS 102 through the network 110. In both of these cases, the MIQA server 104 may receive data from other sources such as an Electronic Medical Records (EMR) system, which may include or be part of the RIS and worklist services software 106, for data related to the patient and study before performing any image assessment. For example, patient data on their height and weight and whether there are any other known conditions (e.g., existing masses) might be used in image assessment.

The MIQA server 104 allows for medical imaging-based quality assurance during various stages of the interpretation and quality assurance processes at a medical institution. For example, the MIQA server 104 runs software programs that allow for the selection or automatic viewing of image quality data of medical imaging studies for Visual Quality Review (VQR) at the time of interpretation via Context Sharing, or by querying databases such as the databases 230 after interpretation, random review of MIT performance (based on input parameters from a user), or automated notifications and/or automated additions to imaging study worklists based on criteria that are set using image quality features or a study level quality score where these criteria are pre-determined and user-configurable. Image quality features and study level quality scores are two examples of image quality data, which is explained in further detail with respect to FIG. 2. A summary of the image-based QA activities for a particular organization and date range can also be automatically generated into a pre-formatted automated report for the purposes of demonstrating and/or maintaining an effective QA system.

When the MIQA server 104 is used during VQR, a database or other data store is updated to record all data and information related to the VQR. Accordingly, the performance of VQRs can be electronically documented, standardized and tracked where a given VQR has a status of pending, active, complete, archived, or deleted. When implementing a VQR using image quality data obtained by the MQA server 104, one or more GUIs can be pre-populated with certain image quality data which may be AI-derived. This streamlines the review completion for reviewers. A reviewer can choose to complete a review, requiring no further action, or they can choose to initiate a review follow-up that places greater scrutiny on the performance of the MIT who acquired the images that are being assessed in the VQR.

The VQRs that are identified for follow-up are electronically documented (e.g., tagged or flagged) to have an ‘active status’ and relevant image quality data is retrieved from a database or datastore to inform any follow-up actions triggered by the VQR. Such image quality data may include automated quality assessments for all images performed by a given MIT over a given period of time. In another embodiment, the Reviewer can alternatively or additionally initiate a subsequent random VQR for the given MIT. It is also possible for other Reviewers to engage with the active VQR. In some embodiments, additional information about corrective actions taken to improve MIT performance can be documented as part of the VQR follow-up. Reviewers can also sign off when they believe sufficient improvements on the part of the MIT have been made.

Accordingly, the MIQA server 104, in one or more embodiments, may execute software that provides functionality and benefits for medical organizations that include at least one of:
comprehensive, standardized, on-demand and continual quality assurance for entire populations (which is otherwise infeasible if solely done manually);
complete replacement of paper-based systems with an electronic software system and database;
AI-based image analysis which reduces the significant burden of image evaluation with reviewers overseeing MIT performance changes and measured responses to corrective actions;
recommendations of corrective actions to assist with performance improvements; and
automated reporting of results that may be used for various purposes such as MIT performance review and/or accreditation/inspection.

Accordingly, the MIQA server 104, in one or more embodiments, may execute software that provides functionality and benefits for various individuals concerned with imaging quality control at medical organizations including at least one of:
for the IP: a reduced administrative burden of overseeing a paper-based QA system, increased efficiency & reduced effort to complete VQRs, and/or a fulfillment of obligations under national regulatory requirements;
for the QC Manager: a standardized system for managing image quality at imaging facilities in preparation for quality inspections, and a software system that monitors IPs and MITs on image quality;
for the MIT: comprehensive and timely performance feedback based on complete body of work, and fulfillment of obligations under national regulatory requirements; and
for the Patient: improved confidence in a system that monitors the quality of every imaging study (including theirs), and not just a handful of image studies once a year.

The MIQA server 104 may execute software that is implemented using a web technology and data analysis technology stacks. In some cases, the MIQA server 104 can be scaled to add multiple discrete servers, by replicating some or all of the discrete services provided by a single MIQA server, across the multiple discrete servers. In this case, the databases used by MIQA software may be hosted across multiple servers, and similarly, the data files may be spanned across multiple servers to provide increased capacity. In some instances, the network bandwidth and/or processing requirements of the MIQA software that is used to perform image quality analysis may be replicated on multiple servers, and results of the analysis transmitted to other MIQA component servers via the network. These scaled solutions may be used for serving a larger medical organization with multi-jurisdictional deployments that may be province-wide, state-wide or nation-wide. In such cases, the MIQA server 104 can be replicated, and they may all push the image quality data into a single MIQA instance.

The RIS and worklist services software 106 are various programs that can be used for managing data and work tasks. For example, the worklist services software can be used to manage the workflow for an IP (e.g., a radiologist) by automatically organizing reading tasks for the IP. The worklist services software includes software instructions for managing the list of image studies that require reporting by radiologists. When there is a specific item of work (e.g., a report write-up for an image study) that must be completed by a radiologist, the worklist services software includes software instructions for tracking this. The RIS is a networked software system that can be used to manage medical imaging operations and associated data, including the archival of completed radiology reports and/or the capture (e.g., receival and recordal) of the reports generated by radiologists. When a radiologist has completed a piece of work, that report is sent to the RIS (and the RIS may forward that report to other systems, such as the MIQA server 104). The RIS and worklist services software 106 can electronically communicate with the reporting services software 108 through the network 110.

The image viewing software 107 may be implemented using various software packages, such as those that are commercially available. The image viewing software 107 allows the IP, LIP, QC manager or other reviewer to retrieve image studies from the PACS 102 and review the medical images of the image study on one of the workstations 112a to 112n. The image viewing software 107 may provide large, high resolution views of the medical images on the workstations 112a to 112n. Conventionally, the image viewing software 107 does not provide any information on image quality for the medical images that are being displayed.

The reporting services software 108 can be used to report the interpretation of medical imaging studies using text or other data elements. Accordingly, the IP uses the reporting services software 108 using the workstation 112a and the electronic network 110 to report their interpretation of one or more medical images of one or more medical imaging studies. For example, the reporting services software 108 can communicate with the PACS 102 to retrieve the medical images and allow the IP to view the medical images at their workstation 112a. The reporting services software 108 and the workstation 112a can also communicate with the MIQA server 104 via the network 110 to enable context-sharing so that the MIQA server 104 can provide image quality data in one or more GUIs to the workstation 112a where the image quality data has been determined for the medical images that the IP is viewing at the workstation 112a. This allows the IP to consider image quality data when interpreting medical images and generating reports, which may improve accuracy of the interpretation. The IP can then use the GUI provided by software that executes on the MIQA server 104 to include certain data or instructions in a report such as edited image quality data and/or instructions for other medical personnel, such as the LIP or QC Manager to review the images.

The LIP and QC Manager, via their workstations 112b and 112n, can also communicate with the MIQA server 104 via the network 110. The MIQA server 104 in turn can communicate with the reporting services software 108 and the PACS 102 to provide medical images, related image quality data and previously saved reports to the workstations 112b and 112n. The LIP and QC Manager can then review the medical images, related image quality data and previously saved reports and perform certain functions including revising the reports and/or determining corrective actions for the MIT who obtained the medical images. These various functions are discussed in further detail herein.

The workstations 112a, 112b and 112n are known computer systems that are used in medical imaging to allow the viewer, such as the IP, LIP or QC, to view larger higher resolution versions of the medical images that are retrieved from the PACS 102. Through context sharing, the MIQA server 104 can provide image quality data on a common display or another display at the workstations 112a, 112b and 112n to allow the image quality data to be considered at the same time as the medical images are viewed. This may be done using Context sharing software. In fact, the context sharing software can be used by the MIQA server 104 to communicate with the PACS 102 or the reporting services software 108 over the network 110.

In at least one embodiment, context sharing can be implemented by providing the MIQA server 104 with a listening service. For example, the image viewing software 107 that is used at the workstations 112a, 112b and 112n to view the medical images can be configured to send an electronic message over the network 110 to the MIQA server 104, whenever a new image study is retrieved and viewed at a workstation. This electronic message may include the identity of the reviewer, and the identity of the image study that was opened. When this electronic message is received by the MIQA server 104, the MIQA server 104 checks if the IP, LIP, QC manager or another reviewer has an open network (e.g., web/Internet/client) session, and if so, updates the session to display image quality data for the most recently opened image study at the corresponding workstation. Sometimes, the IP may retrieve and open the image study using the reporting services software 108, instead of the image viewing software 107. In this case, the reporting services software 108 sends the aforementioned electronic message to both the image viewing software 107, and the listening service of the MIQA server 104. The image viewing software 107 opens the desired image study and the software executed by the MIQA server 104 retrieves image quality data that corresponds to the image study and displays the image quality data in a GUI at the workstation 112a. The image viewing software 107 and the reporting services software 108 have tools that can be configured to notify compatible listeners with status updates, via the electronic messages, for when an image study is being retrieved and viewed at a workstation. The implementation of the status updates and transmission of the electronic messages over the network 110 may be done using standard software instructions such as by using HL7 messages, FHIR-compliant messages or JSON objects, for example, or by providing a REST API implementation that can receive an appropriate custom HTTP request, for example.

The LIP may use the workstation 112b to perform various aspects of the medical facility's quality assurance program to make sure that all compliance requirements are met. Accordingly, the LIP can view various medical images using the workstation 112b to review the performance of a MIT or an IP. In accordance with the teachings herein, the MIQA server 104 may execute software that provides various functions to allow the LIP to view image quality data while reviewing the medical images that have been obtained by a given MIT and/or interpreted by an IP so that the LIP can more easily fulfil the compliance requirements.

The QC manager may use the workstation 112n to perform various aspects of the medical facility's quality assurance program that are not assigned to the LIP. Accordingly, the QC manager can also view various medical images using the workstation 112n to review the performance of a MIT, an IP or even an LIP. In accordance with the teachings herein, the MIQA server 104 may execute software that provides various functions to allow the QC manager to view image quality data while reviewing the medical images that have been obtained by a given MIT, interpreted by an IP and/or reviewed by an LIP so that the QC manager can more easily fulfil the compliance requirements that are assigned to them.

The network 110 can include wired and/or wireless communication hardware that employs communication software for implementing communication protocols to allow the various devices and software systems of FIG. 1 to electronically communicate with one another. The communication hardware, communication software and communications protocols employed by the network 110 are known to those skilled in the art.

Referring now to FIG. 2, shown therein is a block diagram for an example embodiment of a MIQA server 200 that can be used with the system 100 of FIG. 1. The MIQA server 200 may be implemented using a suitable computing device and generally includes a processor unit 202, a display device 204, a network unit 206, I/O hardware 208, a power supply unit 210, and a memory unit 212 that can communicate using a bus 214 and can receive power from voltage rails 216 that are provided by the power supply unit 210. In alternative embodiments some of these elements may not be used. The MIQA server 200 executes software programs that enable to the MIQA server 200 to determine or obtain image quality data for one or more medical images and to display the image quality data to a MIT, IP, LIP, QC manager or other reviewer using one or more GUIs which facilitate various activities during medical image interpretation, medical image review or during various QA activities.

The processor unit 202 may include one processor, for example. Alternatively, there may be a plurality of processors that are used by the processor unit 202 and these processors may function in parallel and perform certain functions. The processor unit 202 controls the operation of the MIQA server 200. The processor unit 202 can be any suitable processor, controller or digital signal processor that can provide sufficient processing power depending on the configuration and operational requirements of the MIQA server 200. For example, the processor unit 202 may include a high-performance processor.

The display device 204 may be used to view a standard video output such as VGA or HDMI. The display device 204 can be any suitable display hardware that provides visual information depending on the configuration of the MIQA server 200. For instance, the display device 204 may be, but is not limited to, a computer monitor, an LCD display, or a touch screen depending on the particular implementation of the MIQA server 200. In some cases, the display device 204 may be used to provide one or more GUIs through an Application Programming Interface or a Web-based application that is accessible via the network unit 206. A user may then interact with one or more GUIs for configuring the MIQA server 200 to operate in a certain fashion.

The network unit 206 includes hardware that allows the processor unit 202 to send and receive data to and from other devices or computers. Accordingly, the network unit 206 includes various communication hardware for providing the processor unit 202 with an alternative way to communicate with other devices. For example, the communication hardware may include a network adapter, such as an Ethernet or 802.11x adapter, a modem or digital subscriber line, a BlueTooth radio or other short range communication device, and/or a long-range wireless transceiver for wireless communication. For example, the long-range wireless transceiver may be a radio that communicates utilizing CDMA, GSM, or GPRS protocol according to standards such as IEEE 802.11a, 802.11b, 802.11g, 802.11n or some other suitable standard. In some cases, the network unit 206 can include other connectivity hardware including a serial port, a parallel port and/or a USB port that provides USB connectivity.

The I/O Hardware 208 includes at least one input device and one output device. For example, the I/O hardware 208 can include, but is not limited to, a mouse, a keyboard, a touch screen, a thumbwheel, a track-pad, a track-ball, a card-reader, a microphone, a speaker and/or a printer depending on the particular implementation of the MIQA server 200. In some cases, all of the I/O functions that might be provided by the local I/O hardware 208 and connected devices, might be accessible via the network unit 206, so that the MIQA server 200, can be operated from a remote setting, such as when the MIQA server 200 is physically located in a remote and/or secure data center location.

The power supply unit 210 can be any suitable power source or power conversion hardware that provides power to the various components of the MIQA server 200. For example, in some cases the power supply unit 210 may include a surge protector that is connected to a mains power line and a power converter that is connected to the surge protector (both not shown). The surge protector protects the power supply unit 210 from any voltage or current spikes in the main power line and the power converter converts the power to a lower level that is suitable for use by the various elements of the MIQA server 200. In other embodiments, the power supply unit 210 may include other components for providing power or backup power as is known by those skilled in the art.

The memory unit 212 can include RAM, ROM, one or more hard drives, one or more flash drives and/or some other suitable data storage elements depending on the configuration of the MIQA server 200. The memory unit 212 stores software instructions for an operating system 218, a MIQA application 220, an image quality analysis module 222, a GUI module 224, a recommendation module 226, an I/O module 228, databases 230 and data files 232. Alternatively, or in addition thereto (for backup), the databases 230 and the data files 232 may be stored on separate data stores which may be co-located with the MIQA server 200 or remotely located from the MIQA server 200. The various software instructions, when executed, configure the processor unit 202 to operate in a particular manner to implement various functions and tools for the MIQA server 200. For example, the operating system 214 includes software instructions for operating a computing device, such as the MIQA server 200, as is known by those skilled in the art.

The MIQA application 220 includes software instructions, that when executed by the processor unit 202, configures the MIQA server 200 to provide various functions during medical image interpretation and various medical image quality assurance operations. Examples of the functions provided by the MIQA application 220 are methods 300, 400, 500, 600, 700, and 800 described herein. In providing the functionality of methods 300 to 800, the MIQA application 220 can configure the processor unit 202 to execute the software instructions of the image quality analysis module 224 for determining and/or obtaining image quality data for one or more medical images that are being viewed on one of the workstations 112a to 112n. The MIQA application 220 can also configure the processor unit 202 to execute the software instructions of the GUI module 224 for providing various GUIs to display certain image quality data and receive commands and input data from a user of one of the workstations 112a to 112n. Examples of the GUIs that may be used during the operation of any of the methods 300 to 800 are provided herein. The MIQA application 220 may also configure the processor unit 202 to execute the software instructions of the recommendation module 226 when performing certain functions in order to provide recommendations to the user of one of the workstations 112a to 112n for taking certain actions.

The image quality analysis module 222 includes software instructions that, when executed by the processor unit 202, configure the processor unit 202 to determine image quality data for medical images in situations where the image quality data has not been previously determined. The determined image quality data is then saved on the databases 230. Alternatively, for situations where the image quality data has already been determined for medical images that are being viewed at one of the workstations 112a to 112n, the image quality analysis module 222 can be used to retrieve the required image quality data from the databases 230. The type of image quality data that is retrieved depends on the GUI that is being used to display the image quality data on one of the workstations 112a to 112n.

The term image quality data can include various image quality scores or indices that may be defined for various parameters. For example, the term “Image Quality Parameter (IQP)” is used to refer to a feature or metric that is developed to identify a particular non-conformity that may be found in a type of medical image, such as a digital mammogram including “for processing” and “for presentation” mammograms. A list of example IQPs is provided in Table 1. It is understood that there may be other IQPs used by the example embodiments described herein, and variations thereof, and Table 1 is provided as an example and is not necessarily exhaustive.

TABLE 1
Some Example Image Quality Parameters (IQPs)

| IQP | Description |
|---|---|
| CC Exaggeration | Nipple is not in midline of the imaged breast |
| Portion Cut-off | Parts of the breast cut off by the edge of the |
| | image |
| Skin Folds | Skin folds visible |
| Posterior Tissues | Posterior breast tissue is not imaged |
| Missing | |
| Inadequate IMF | Inframammary fold not clearly seen or not imaged |
| Pectoralis Muscle | Shape of the pectoralis muscle in the MLO view |
| Concave | is not straight or convex |
| MLO Sagging | Breast orientation exhibits sagging in MLO view |
| MLO too high on IR | Breast is positioned too high on Image Receptor |
| IMF Missing | Inframammary fold, where the lower portion of the |
| | breast and chest meet, is not visible on the image |

The term “Image Quality Parameter Score” (IQPS) may be used to refer to image quality data that provides a predicted probability of the presence of an error for a given IQP in a medical image. In addition, an Image Quality Parameter Index (IQPI) and/or a distinct predicted class, which may also include a corresponding confidence for the class, may both be generated based on one or more Image Quality Parameter Features (IQPFs) and IQPFs. An IQPF represents a measurement of some aspect of the medical image that is directly or indirectly related to an IQP. A list of example IQPFs is provided in Table 2. It is understood that there may be other IQPFs used by the example embodiments described herein, and variations thereof, and Table 2 is provided as an example and is not necessarily exhaustive. The IQPFs are described in detail in published PCT application WO 2020/102914.

TABLE 2
Some Examples of Image Quality Parameter Features (IQPFs)

| Feature Name | Description |
|---|---|
| Compression Force | DICOM meta data: Compression force applied by |
| | imaging device (reported) |
| Compressed Breast Thickness | DICOM meta data: Thickness of breast reported |
| | by imaging device (reported) |
| Breast Area | Area of breast visible in the image (computed) |
| Compression Pressure | Compression pressure applied by imaging device = |
| | compression force/breast area (computed) |
| Breast Volume | Volume of breast = breast thickness * breast area |
| | (computed) |
| Patient Age at Mammography | DICOM meta data: Age |
| PNL_Angle | MLO - Angle of PNL line from a horizontal |
| | reference |
| PNL | MLO - Length of PNL |
| PNL_C | MLO - the horizontal distance from an intersection |
| | point to image edge |
| PNL_D | MLO - the distance from intersection point to |
| | image edge on the extension of the PNL |
| PNL_E | MLO - the distance along image edge, between |
| | the points where the PNL intersection's horizontal |
| | and the PNL extension line |
| PNL_F | MLO - PNL does not intersect the pectoralis |
| | muscle, and the distance of the end of the |
| | pectoralis muscle and the PNL |
| saggingPnlAngle | MLO - an angle of the nipple location to midpoint |
| | of posterior breast |
| saggingTissueAngle | MLO - an angle of the line formed from the nipple |
| | to the mid-point of posterior breast |
| pecWidth | MLO - Width in mm of the pectoralis muscle at top |
| | edge of MLO view |
| concavePecAngleDiff | MLO - a Difference between angles from the line |
| | segment aligned with the first superior one-third of |
| | the pec muscle, and the line segment aligned with |
| | the second superior one-third of the pec muscle |
| inadequateIMFCurve | MLO - Maximum curvature of skin line that forms |
| | the IMF |
| inadequateIMFHeight | MLO - Vertical distance of IMF location from |
| | bottom edge of image |
| inadequateIMFWidth | MLO - Horizontal distance of IMF location from the |
| | posterior edge of the image |
| tooHighOnIRImfRatio | MLO - Ratio of the IMF to whole vertical size of the |
| | breast (i.e., Ratio of distance from IMF to superior |
| | image edge, to the vertical length of the breast |
| | area) |
| tooHighOnIRNippleRatio | MLO - Ratio of the nipple to the whole vertical size |
| | of the breast (i.e., ratio of nipple distance to inferior |
| | image edge, to the vertical length of the breast |
| | area) |
| skinFoldsNumber | MLO - Number of bright blobs in the region near |
| | the IMF and the breast axillary tail where the axilla |
| | is closest to the topmost part of the breast in the |
| | MLO view |
| skinFoldsSize | MLO - Total size (i.e., sum of area) of bright blobs |
| | found in regions near IMF and breast axillary tail |
| posteriorTissuesTissueDensity | MLO - Statistics of the pixel intensity from dense |
| | tissue |
| posteriorTissuesTargetDensity | MLO - Statistics of pixel intensity calculated from |
| | rectangle target region inside the breast region, |
| | below pectoralis muscle and close to chest wall |
| cutOff | CC/MLO - Test if breast area touches edges of |
| | image |
| PNL | CC - Length of PNL Horizontal distance from |
| | nipple to pec muscle in CC view |
| CCExaggerationSymmetry | CC - Dividing by PNL, the symmetry measurement |
| | of medial and lateral portion of breast (i.e., |
| | difference between length of breast area on |
| | medial and lateral sides of the PNL, normalized by |
| | PNL length) |
| CCExaggerationAngle | CC - Angle of midpoint of the posterior breast to |
| | the nipple |
| CCExaggerationNipple | CC - Ratio of the nipple location to the whole |
| | vertical breast length |
| posteriorTissuesTissueDensity | CC - Statistics of the pixel intensity from dense |
| | tissue |
| posteriorTissuesTargetDensity | CC - Statistics of pixel intensity are calculated from |
| | rectangle target region inside breast region and |
| | close to chest wall |
| skinFoldsNumber | CC - Number of bright blobs found in regions near |
| | medial and lateral end of the breast |
| skinFoldsSize | CC - Total size of bright blobs found in regions |
| | near medial and lateral end of breast |
| imageHeight_MLO | MLO - Total vertical length of MLO image |
| imageHeight_CC | CC - Total vertical length of CC image |
| imageWidth_MLO | MLO - Total horizontal length of MLO image |
| imageWidth_CC | CC - Total horizontal length of CC image |
| breastWidth_MLO | MLO - distance from most anterior point on breast |
| | contour to anterior edge of MLO image |
| breastWidth_CC | CC - distance from most anterior point on breast |
| | contour to anterior edge of CC image |
| oppositeBreastWidth_CC | CC - imageWidth subtract breastWidth which is |
| | farthest anterior point of skin line to the anterior |
| | edge of the CC image |
| oppositeBreastWidth_MLO | MLO - imageWidth subtract breastWidth which is |
| | farthest anterior point of skin line to the anterior |
| | edge of the MLO image |
| lengthFromBottom_CC | CC - Length from most medial point on breast |
| | contour to medial edge of CC image |
| lengthFromBottom_MLO | MLO - Length from most inferior point on breast |
| | contour (excluding IMF) to inferior edge of MLO |
| | image |
| cutOffLength_A_CC | CC - Length of intersection between breast and |
| | image edge, when cut off is detected on anterior |
| | edge of CC image |
| cutOffLength_M_CC | CC - Length of intersection between breast and |
| | image edge, when cut off is detected on medial |
| | edge of CC image |
| cutOffLength_L_CC | CC - Length of the intersection between breast |
| | and image edge, when cut off is detected on |
| | lateral edge of CC image |
| cutOffLength_A_MLO | MLO - Length of intersection between breast and |
| | image edge, when cut off is detected on anterior |
| | edge of MLO image |
| cutOffLength_I_MLO | MLO - Length of intersection between breast and |
| | image edge, when cut off is detected on inferior |
| | edge of MLO image |
| lengthFromTop_CC | CC - Length from most lateral point on breast |
| | contour to lateral edge of CC image |
| inadequateIMFWidth_I_MLO | MLO - Horizontal distance of most inferior portion |
| | of IMF to posterior edge of MLO image |
| nipplePosition_A | MLO - Length from nipple to anterior edge of MLO |
| | image |
| nipplePosition_P | MLO - Length from the nipple to posterior edge of |
| | MLO image |
| nipplePosition_I | MLO - Length from nipple to inferior edge of MLO |
| | image |
| fromBottomNippleRatio | MLO - Ratio of lengthFromBottom to |
| | nipplePosition_I (calculated) |
| pecBreastWidth | MLO - Length of intersection between breast and |
| | superior edge of MLO image |
| thicknessRatio | MLO - Ratio of the breastWidth to pecBreastWidth |

The same terminology can be expanded to an overall image quality score, and/or an Image Quality Index (IQI) for individual medical images or medical images that are part of an image study. The IQI can be generated from one or more IQP scores in that the predicted image quality parameter scores may then be combined to determine an image quality index and/or image level classification.

These aforementioned scores are derived for image quality parameters from the image data, and at least one of the metadata information and the clinical patient data. These parameters may also include the positioning parameters of the patient 120 based upon their location at the medical imaging device 116 during image collection/acquisition, physical parameters of the patient, and image quality parameter features. The parameters may correspond to known deficient conditions or non-conformity in the mammographic images. For example, a particular parameter ‘posterior tissues missing cc’ may have a predicted numerical value that is between 0 and 100. Indexing of the score for the parameter ‘posterior tissues missing cc’ may produce an indexed prediction of “Bad” for a range 90-100, “Acceptable” for a range of 50-90, “Good” for the range of 20-50 and “Great” for the range of 0 to 20. This is just one example of the indexing that may be done.

The various image quality scores are predicted in the sense that a predictive model is used to determine the score which may involve inputting covariates into the predictive model and the predictive model predicts the probability of the event (which is the presence of an image quality error amongst the plurality of image quality errors). The predicted image quality parameter scores can be considered to correspond to the probability that the non-conforming conditions that correspond to those parameters exist in a given mammographic image. Likewise, the predicted study quality parameter scores may correspond to the probability that the non-conforming conditions that correspond to those parameters exist in the study (e.g., set of images).

An overall predicted image quality score may be a gestalt measure and may use as inputs the IQPFs and the IQPSs. An IQI is a mapping from a predicted image quality score to a discrete, categorical or ordinal scale. The indexing may be performed based on statistical regression or machine learning using supervised or unsupervised approaches. The IQI may provide concrete decision points. For example, an MIT may decide to perform a mammographic image collection a second time to resolve non-conforming conditions based on the IQI and/or indexed image quality parameter scores. The image quality score or an image quality index may be a decimal number between 0 and 1. Alternatively, the image quality score or the image quality index may be expressed, for example, as pass/fail classifications of the image quality for one or more images. Similarly, the indexing may be for non-binary classifications, such as “perfect”, “good”, “moderate” and “inadequate”.

Likewise, the image quality image data may include a plurality of predicted study quality parameters, a corresponding plurality of predicted study quality parameter scores, and/or an overall predicted study quality score. Alternatively, or in addition thereto, for either the overall image quality assessment or the overall study quality assessment, the image quality data may also include a predicted class that is generated by a classifier model based on the predicted image/study quality parameter scores.

The aforementioned scores, parameters and indices can also be determined for all of the images in an image study for determining a plurality of predicted study quality parameters, a corresponding plurality of predicted study quality parameter scores, a plurality of a predicted study quality parameter features, a plurality of study quality parameter feature scores, a plurality of study quality parameter indices, and/or an overall predicted study quality score/index. For example, a study quality parameter feature represents a measurement of some aspect of the images in an image study which affects the overall image study quality. A list of example SQPFs is provided in Table 3. It is understood that there may be many other SQPFs used by the example embodiments described herein, and variations thereof, and Table 3 is provided as an example and is not necessarily exhaustive.

### Table 3—Some Examples Study Quality Parameter Features (SQPFs)

TABLE 3
Some Examples Study Quality Parameter Features (SQPFs)

| Feature Name | Description |
|---|---|
| PNL length difference | Difference in the length of |
| | the PNL as measured in the |
| | CC and MLO views for one breast. |
| Tissue cutoff | Test if a study that includes |
| over all | multiple MLO images for a |
| MLOs per side | single side, has tissue cutoff |
| | on either the superior (top) |
| | or inferior (bottom) edges. |
| Tissue cutoff over all | Test if a study that includes |
| CCs per side | multiple CC images for a |
| | single side, has tissue cutoff |
| | on either the medial (bottom) |
| | or lateral (top) edges. |
| Posterior tissues | Test if a study has posterior |
| missing in both CC and | tissue missing in both the CC |
| MLO per side | and MLO images for a single side. |
| Portion Cut-off overall | Parts of the breast cut off |
| | by the edge of the image not |
| | visible in any of multiple repeated views. |
| Posterior Nipple Line | Distance from nipple to |
| | pec. muscle along a line |
| | perpendicular to pec muscle, |
| | are not <1 cm of each |
| | other, when measured in |
| | CC and MLO views. |
| Posterior Tissues | Posterior breast tissue not |
| Missing Overall | visible in either CC or MLO |
| | view of the breast. |

The overall image study quality score can be derived from a model that takes IQPFs, IQPSs, IQPIs, SQPFs, SQPSs, and SQPIs as inputs. The study quality score may reflect the predicted probability of non-conformity for the plurality of images in the image study, a minimum of the IQPs for the plurality of images in the study, a maximum of the IQPs for the plurality of images in the study, or another statistical summary measure of the underlying IQPs of the images that are part of the study. The predicted study quality scores may be combined to determine a gestalt (or overall) study quality index and/or study quality classification.

Techniques for determining the various types of image quality data described above are provided in PCT application publication WO 2020/102914.

While the image quality analysis module 222 is described herein as operating on standard “for presentation” mammographic images as recorded by the MIT 118, it should be understood that the methodology described herein for using various image and study parameters, determining scores comprising predicted probabilities for those parameters, and generating an index and/or a classification, may be done on medical images that are collected/acquired on film and then digitized as well as raw medical images also known as “for processing” mammographic images.

The GUI module 224 includes software instructions that, when executed by the processor unit 202, configure the processor unit 202 to provide a visual display of image quality data, messages and/or reports to a user of one of the workstations 112a to 112n or another computing device that can access the medical imaging system 100 according to a certain layout for each user interface and also to receive inputs from the user. The GUI module 224 includes software instructions that, when executed by the processor unit 202, configure the processor unit 202 to change the image quality data that is shown on the current GUI depending on when the user is editing the image quality data or shows a different GUI depending on the user inputs.

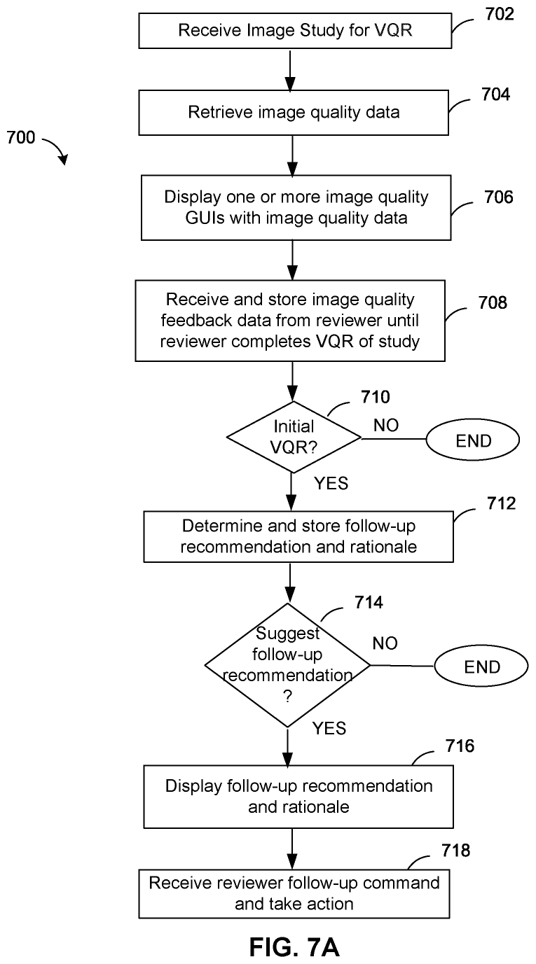

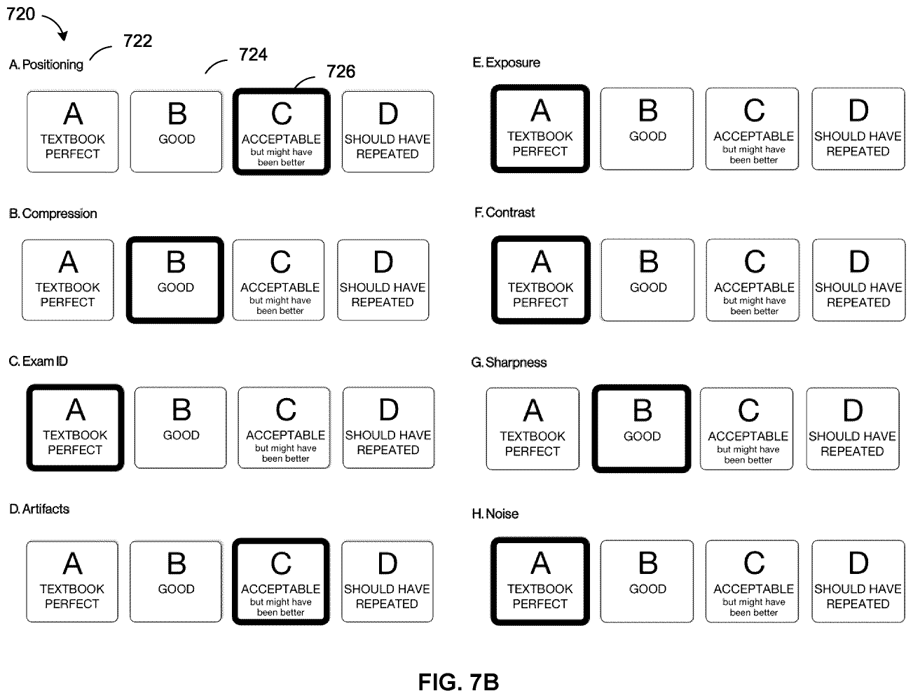

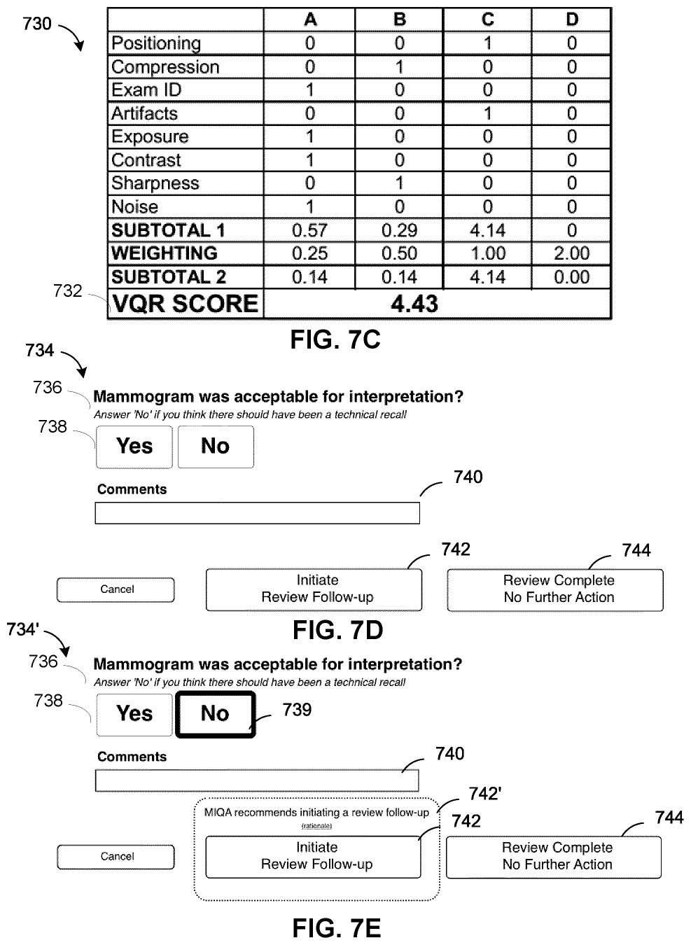

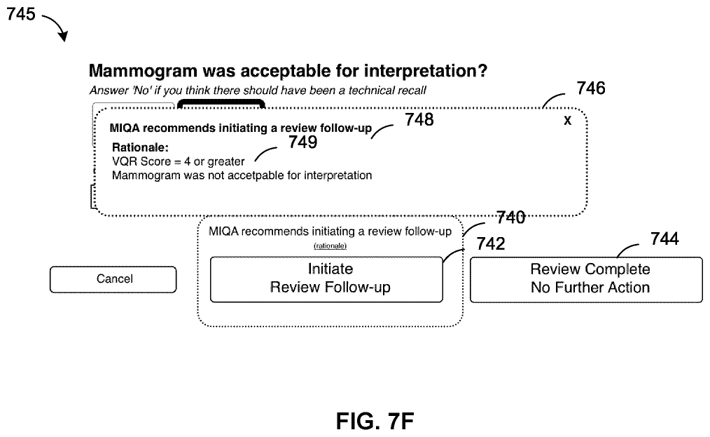

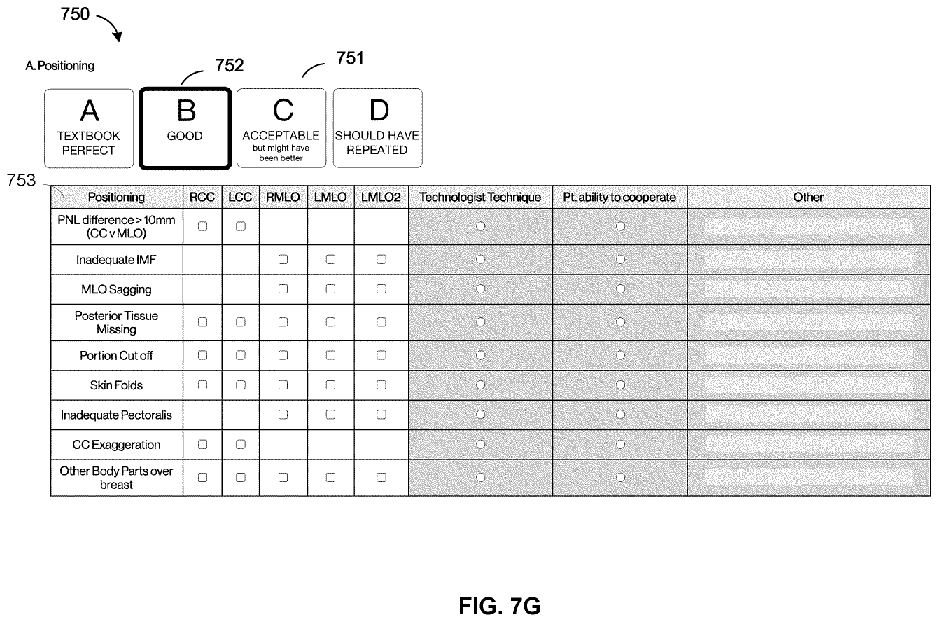

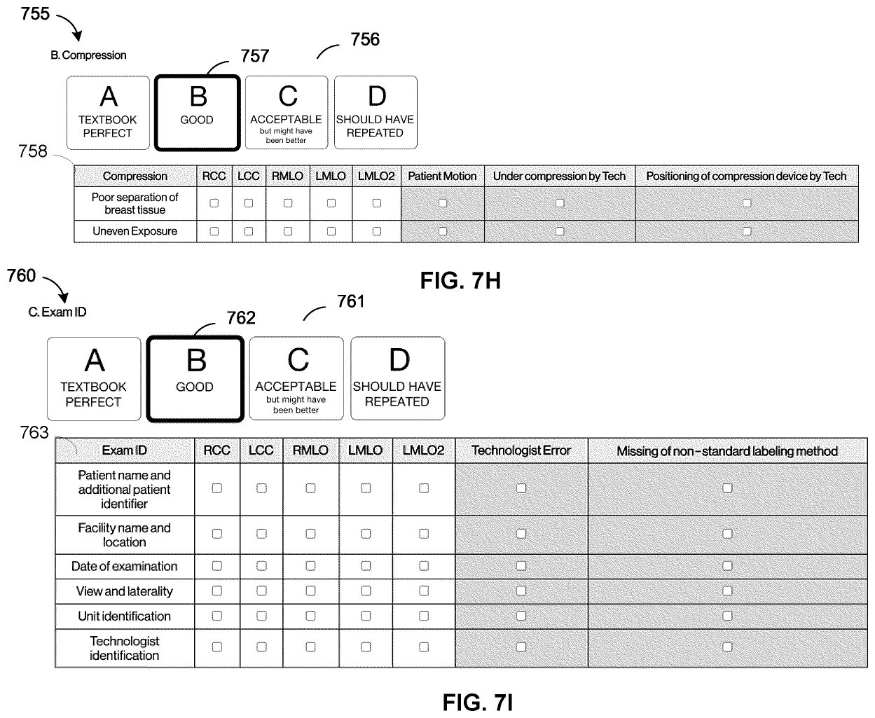

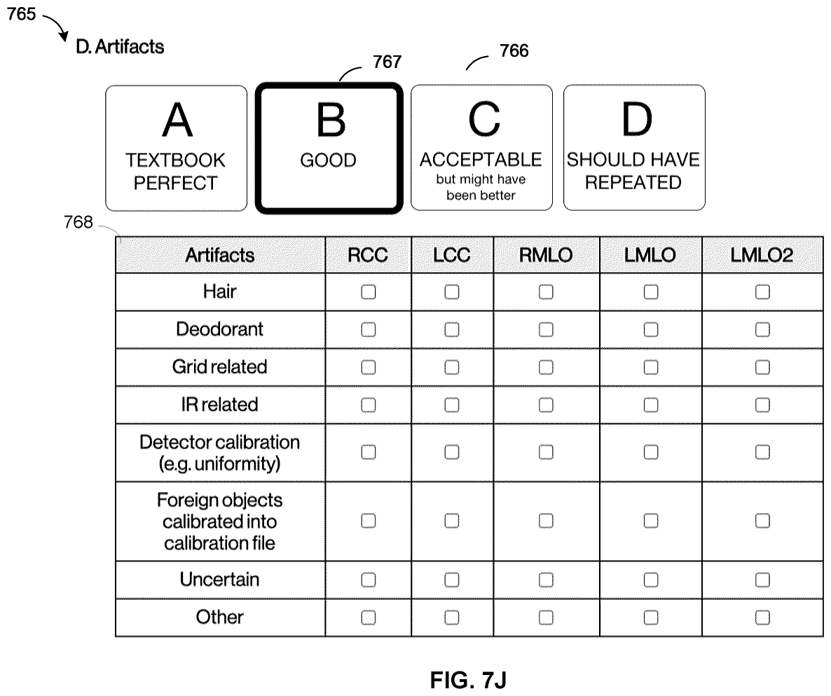

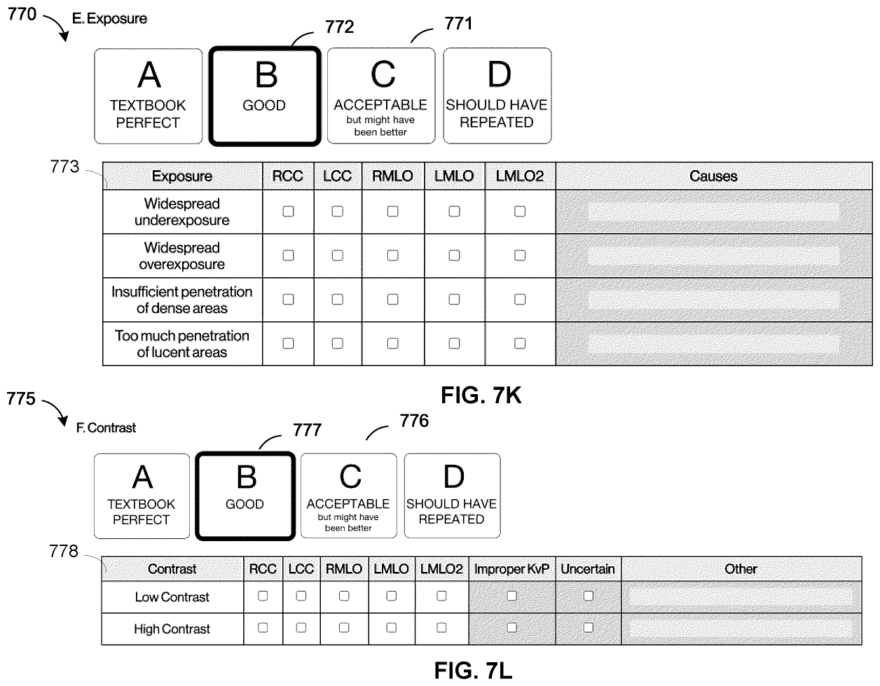

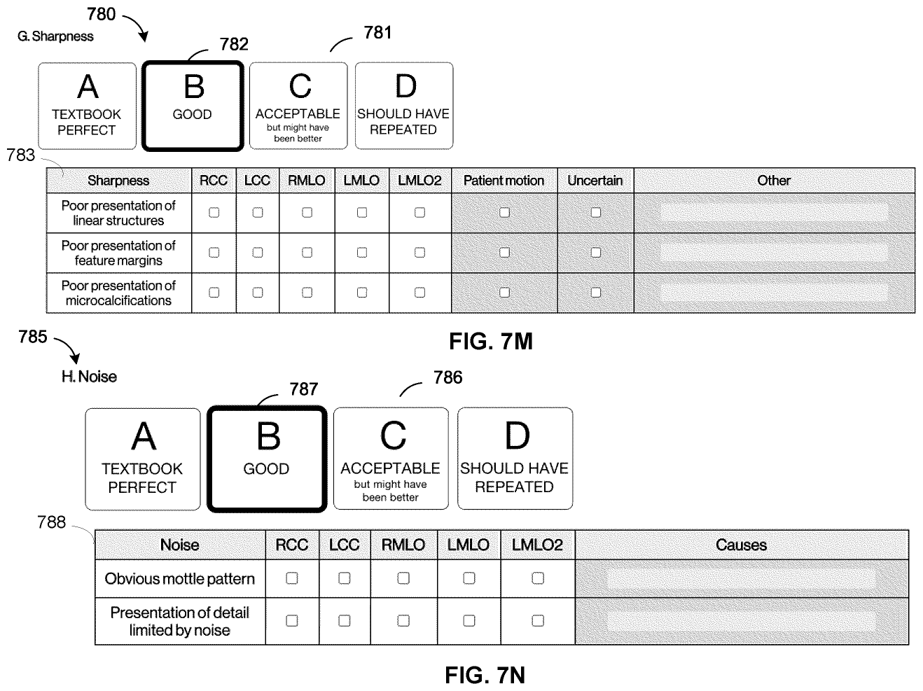

The recommendation module 226 includes software instructions that, when executed by the processor unit 202, configure the processor unit 202 to generate certain recommendations depending on which functions are being provided by the MIQA application 220. For example, the recommendation module 226 can include software instructions for providing recommendations to a reviewer, such as an IP, that VQR should be performed on an image study, an example of which is described with respect to method 400 and FIGS. 4A to 4E. Alternatively, or in addition thereto, the recommendation module 226 can also include software instructions fora reviewer follow-up, an example of which is described with respect to method 700 and FIGS. 7A to 7N.

The I/O module 228 includes software instructions that, when executed by the processor unit 202, configure the processor unit 202 to store information in the databases 230 and/or data files 232 or retrieve data from the databases 230 and/or data files 232. For example, any input data that is received through one of the GUIs can be stored by the I/O module 228. In addition, any image quality data that is required for display on a GUI may be obtained from the databases 230 using the I/O module 228 or any operational parameters that are needed for provision of any of the functions provided by the MIQA application 220 may be obtained from the data files 232 using the I/O module.

The databases 230 may be used to store a plurality of image quality data that correspond to various medical images and/or image studies that are stored on the PACS 102. The databases 230 can also store certain image metadata that may be used in obtaining the image quality data. In some embodiments, the databases 230 may also be used to store other measures that are obtained from the medical images and the metadata such as breast density and/or breast cancer risk prediction. The breast density may be determined according to various techniques such as, but not limited to, techniques described in U.S. Pat. No. 9,895,121, which is hereby incorporated by reference in its entirety or techniques that are described in PCT application publication WO 2020/102914. The breast cancer risk prediction can be determined according to various techniques such as, but not limited to, techniques described in by Abdolell et al. (Abdolell 2020) and/or Yala et al. (Yala 2019), both of which are incorporated herein by reference in their entirety.

The data files 232 may be used to store predictive models that are used to determine the image quality data and other parameter values and control settings that are used to determine the image quality data as explained in PCT application publication WO 2020/102914. The data files 232 may also be used to store temporary data which is used during the operation of the MIQA application 220, the image quality analysis module 222, the GUI module 224 and/or the recommendation module 226. The data files 232 may also be used to store copies of the medical images for presentation alongside the quality results. In some cases, lower-fidelity, compressed versions of the medical images, that are still clearly recognizable when compared to the originals, may be used for this purpose.

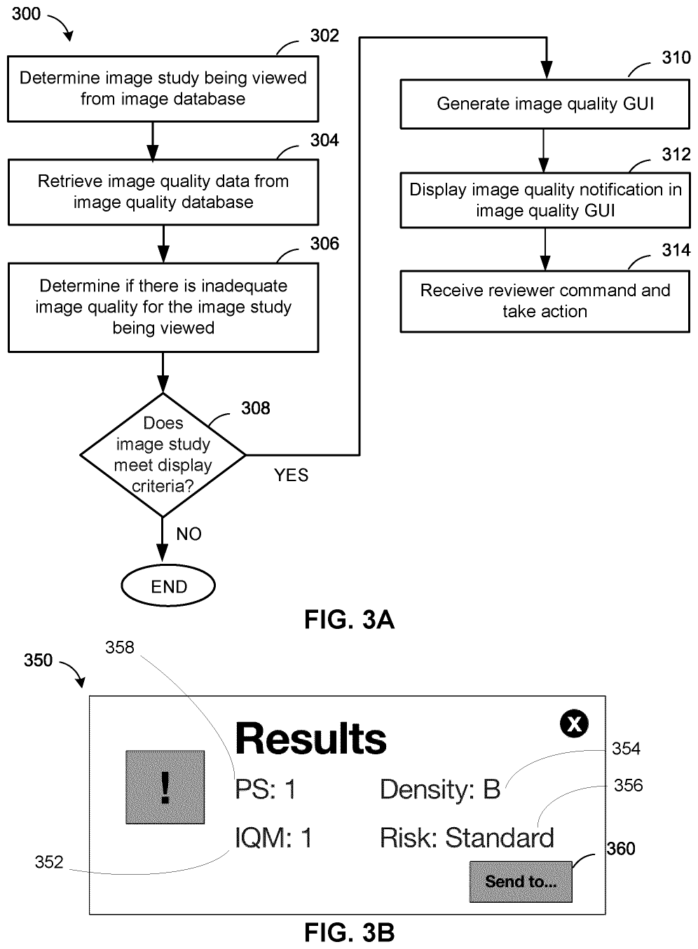

Referring now to FIG. 3A, shown therein is a flow chart of an example embodiment of a method 300 for notifying an IP when the image quality assessment, via the image quality data, for an image study that is being viewed has inadequate image quality. In particular, the method 300 triggers the automatic display of a pop-up window (see FIG. 3B for an example), or another electronic message, including at least one measure related to the image study that is being viewed by the IP at the workstation 112a or another computing device. This can be done automatically without the need for the IP to send an electronic query to the MIQA server 200 to fetch the image quality data. Accordingly, the method 300 allows for a context sensitive review of at least one image or an image study, where associated quality data is automatically provided to the reviewer. While the method 300 is described with respect to when an IP is viewing an image study, it can be performed when any reviewer is reviewing an image study. The method 300 may be performed by a processor of the processor unit 202 of the MIQA server 200 of FIG. 2.

Referring now to the example embodiment shown in FIGS. 3A-3B, method 300 begins with act 302 where it is determined, through context-sharing for example, that an image or an image study is being retrieved from an image database (e.g., the PACS 102) for viewing on one of the workstations 112a to 112n or another computing device that can access the medical imaging system 100. For example, the MIQA server 200 can receive an indication that an image study is being retrieved for viewing at a workstation (which can be considered to be a computing device) by a user (e.g., IP or other reviewer) and an image study ID for the image study. At act 304, the method 300 involves retrieving image quality data from a database, e.g., an image quality database, such as one of databases 230 or another data store, where the image quality data that was determined for the image study being viewed is stored. The image quality data that is retrieved may be an image quality metric that summarizes or provides an overall indication of the image quality of the various images in the image study. An example of such an image quality metric is an image quality index. In alternative embodiments, other image quality data can be provided such as any of the image quality errors shown in FIGS. 7E-7M such as compression pressure, posterior tissue missing or IMF missing, or other individual image quality measures. This may be configurable by the end-users according to whether these measures warrant particular attention, so that feedback can be given immediately, or if extra caution should be exercised during interpretation.

At act 306, it is determined if there is inadequate or poor image quality for the image study being viewed, which means that a correct diagnosis may not be determined from the image study. This determination may be done by comparing the image quality index to an image quality criterion, which might be a threshold, for example. At act 308, if the image quality is adequate so that the image study does not require automatic triggering of a notification, then the method 300 ends. Alternatively, if at act 308, the image quality is inadequate then the method 300 proceeds to acts 310 and 312 where an image quality GUI, in the form of a pop-up notification in this example, such as window 350 shown in FIG. 3B for example, is generated and displayed along with at least some of the image quality data and optionally other measures, at the display of the workstation to the IP.

The measures in the GUI window 350 are meant to provide a quick summary of one or more measures of the image study so that is easy for the IP to quickly review. The measures include an indication of image quality of the image study that is being viewed as well as one or more optional additional measures that can aid the IP in interpreting the images in the image study including making a diagnosis. In one example embodiment, the pop-up window 350 may be used to display an image quality index 352, and additional measures including a breast density index 354, a cancer risk index 356, and an overall priority score 358. If the reviewer is interested in reviewing any of the additional measures in more detail this may be done by accessing further data such as breast density data and/or cancer risk index data can be retrieved from a database such as database 230, for example. The MIQA application 220 can determine the priority score, which may be based on a combination of image quality data and other measures, in one example embodiment. In other embodiments, the additional measures can include any combination of the breast density index 354, the cancer risk index 356, and the overall priority score 358. Alternatively, in other embodiments, the additional measures are optional and may not be shown.

The method 300 then receives a command from the IP for performing a follow-up task and takes the corresponding action at act 314. This follow-up action can be electronically recorded and linked with the image study and the image quality data by updating the database 230 used by the MIQA server 200. This is beneficial since conventionally the IP typically writes a note on paper when there is a quality issue that needs to be discussed with the MIT or another reviewer. However, with conventional workflows it is easy for this note to get lost or the IP may forget to send it to the MIT or other reviewer, or for there to be no follow-up undertaken and no way for the IP to be completely certain and aware in a timely manner that follow-up was undertaken and confirmed by a fully traceable audit trail, since the IP's job is very fast-paced and there is a large volume of image studies to review. If the image study is not flagged for poor image quality or this assessment gets lost, then it is assumed that the image study has good image quality which will lead to errors as explained previously. Furthermore, according to conventional practice, the IP assesses the image quality themselves which is somewhat subjective and biased, whereas the image quality data and comparison with image quality criteria that is performed by method 300 provides a more rigorous, standardized way for the IP to decide whether an image study has inadequate image quality and then take follow-up actions which are electronically documented and easily accessible by anyone who can access the MIQA server 200 or other elements of the medical imaging system 100.

The action taken at act 314 is based on a command selected by the IP by pushing the input button 360 which may then open a drop-down window/menu that lists the follow-up tasks. The selected follow-up task is determined based on which one is selected by the IP. The command from the IP is based on reviewing the image quality data and optionally the additional measures. The follow up tasks may include one or more of:
displaying an enhanced view of the image quality data for the image study being reviewed where the enhanced view provides more image quality data that can be reviewed (an example of an enhanced view is shown in FIG. 4B);
using the MIQA application 220 to schedule a follow-up VQR of the image study or more generally for the MIT;
sending an electronic message with a reference to the image study (e.g., the image study ID) to the RIS and/or worklist services software 106 for electronic documentation and creation of a report of the review of the image study including the image quality, optionally the breast density and optionally the cancer risk;
sending an image quality report to the PACS 102 to store that report for presentation and archival alongside the original images of the image study, so that the image quality report can be consistently viewed in line (i.e., at the same time) with the image study images;
sending an electronic notification message to the worklist services software 106 that the image study has inadequate image quality, so that the image study may be prioritized for review, and/or so that the image study's descriptive information in the worklist indicates that the image study may have inadequate image quality;
sending an electronic notification message to the RIS and/or the worklist services software 106, such that another follow-up action may be generated such as to recall a patient (from whom the image study was obtained) and perform further imaging using X-ray or another imaging modality such as ultrasound or MRI; sending an electronic notification message to the Reporting services software 106 to electronically document and include in a report that the image quality for the image study that was viewed is inadequate; and
sending an electronic request message to another user of the MIQA application 220, such as another IP, a LIP or a QC manager, for example, that they should review the image study to provide a second assessment and determine whether they agree with the assessment made by the IP who initially reviewed the image study.

In an alternative embodiment, instead of comparing the image quality data to a criterion, the notification can be automatically displayed whenever an IP, or another reviewer, is viewing an image study on a workstation or other computing device.

Referring now to FIG. 4A, shown therein is a flow chart of an example embodiment of a method 400 for providing image quality data when an IP or another reviewer is reviewing a selected image study. To facilitate review of image studies, upon displaying the image quality data to the IP, the method 400 can prompt the IP for input on whether the IP wants to send the image study for VQR. The method 400 provides this functionality using a single-click, automated process. As with method 300, method 400 can determine when the IP is reviewing an image study and the ID of the image study, through context sharing, and retrieves and displays a more thorough image quality assessment using image quality data that was previously determined for the image study. This allows the IP to review the more detailed image quality data while interpreting the images of the image study in order to make a more detailed interpretation and diagnosis. Conventionally no image quality data is displayed when an IP, or another reviewer, retrieves and displays an image study at a workstation. The method 400 may be performed by a processor of the processor unit 202 in FIG. 2.

In another aspect, in at least one embodiment, the method 400 can also provide a recommendation to the IP, or other reviewer, when an automated recommendation process determines that the image study should most likely be sent to VQR. However, in this example embodiment, the final decision resides with the IP on whether to send the image study to VQR. Accordingly, method 400 allows for the interpretation process to be done in a more standardized way and also electronically captures the image studies that are determined by the IP to require a VQR. A VQR is a thorough quality assessment, that is completed by a qualified reviewer, who reviews various quality criteria (e.g., but not limited to, positioning, compression, exam ID, artifacts, exposure, contrast, sharpness, and/or noise) and decides whether the image study is acceptable for interpretation. While image quality data can be automatically generated using AI machine learning technology, at least one example of which is described herein, visual quality assessments are conventionally performed by human reviewers as part of standard practice.

Method 400 provides useful functionality in assisting the IP to more efficiently and accurately perform their tasks during interpretation of an image study. Conventionally, during interpretation of an image study, an IP has two main tasks: (1) find abnormalities (e.g., cancer) and (2) determine if the medical images in the image study are of sufficient quality to complete task 1 and decide if the medical images should undergo a VQR. Conventionally IPs have assistance from computer aided detection (CAD) software for the first task but conventionally there are no assistive software tools for the second task.

In another aspect, in at least some embodiments, the method 400 enables the IP or another reviewer to modify the previously determined image quality data that is currently being displayed. By allowing IPs and other qualified reviewers to modify the image quality data, which are initially determined using machine learning technology, the aggregate image quality data that is presented for an image study is more accurate, and any modifications can be sent back to the machine learning technology to improve the algorithms that are used for automatically generating the image quality data.

In another aspect, in at least some embodiments, the method 400 can also provide additional measures that were determined for the image study which can aid the IP in interpreting the images of the image study and generate a report for the image study. The additional measures include a breast density data, cancer risk data, priority data, or any combination thereof.

Referring now to the example embodiment shown in FIGS. 4A-4F, method 400 begins at act 402 where it is determined, through context sharing, that a given image study is being retrieved from the PACS 102 for viewing at a workstation or other computing device that can access the medical imaging system 100. The ID of the image study is determined. At act 404, the image study ID is used to retrieve image quality data that corresponds to the given image study from the database 230 or a corresponding data store. In this example embodiment, additional measures are also retrieved that correspond to the given image study. The additional measures include breast density data, cancer risk data and priority data.

In an alternative embodiment, when the image quality data and/or additional measures for the image study that is being viewed have not already been computed, then the image quality data and/or additional measures can be determined by the image quality analysis module 222. As previously described, techniques for determining the various types of image quality data and additional measures are described in PCT application publication WO 2020/102914.

At act 406 an image quality GUI is generated and at act 408, the image quality GUI along with the retrieved or recently generated image quality data, and optionally the retrieved additional measures, are displayed at the workstation/computing device.

An example image quality GUI 430 is shown in FIG. 4B. the GUI 430 shows an identifier 432 that includes data on the patient and the image study for which the image quality data was determined. The identifier 432 can be cross-referenced with an identifier associated with the mammograms being displayed by the image viewing software 107 at the workstation to ensure that the correct image quality data is being displayed. The GUI 430 includes a subwindow (e.g., a region) having a plurality of image quality data and also includes images 434 that are part of the image study that is being viewed at the workstation using the image viewing software 107.

In this example embodiment, the image quality data includes quality data for a plurality of image parameter feature scores 436 which include positioning metrics, breast related metrics, imaging acquisition parameters, and/or DICOM metadata parameter values for the different views of the image study, which in this case are the RCC, LCC, RMLO, RMLO2, LMLO, and LMLO2 views. In this example, there are extra views for two images for the RMLO and the LMLO. In other embodiments, other types of image parameter feature scores and views may be shown. The image parameter feature scores 436 can include a variety of different metrics and is not limited to what is shown in FIG. 4B. In this example, the image parameter feature scores 436 include positioning metrics such as: PNL Difference >10 mm, CC Exaggeration, portion cut-off, skin folds, Posterior Tissue Missing, Inadequate Pec Muscle Length, Pec Muscle Concave, IMF Missing, IMF Inadequate, MLO Sagging, MLO too high on IR, Sharpness, Contrast, Exposure, Noise, Artifacts, and Nipple in Profile. In this example, the breast related metrics include MLO Angle, Breast Volume, and Breast Area. In the GUI, an error symbol (e.g., the X's) indicate that the view contains an error for that metric while a pass symbol (e.g., the -'s) indicate that the view does not contain an error for that metric. Therefore, the more error symbols that are displayed provides a quick visual indication that there are more image quality problems with the image study. Conversely, the more pass symbols that are displayed provides a quick visual indication that there are less image quality problems with the image study. Examples of DICOM metadata parameter values, although not shown in FIG. 4C may include age, sex, weight, imaging machine model, and manufacturer. Further, in this example, the imaging acquisition parameters include radiation dose, compression pressure and compression force. In other embodiments, there may be image quality data for a different combination of positioning metrics, a different combination of breast-anatomy related metrics, a different combination of imaging acquisition parameters, and a different combination of DICOM metadata parameters. In other embodiments one or more of the positioning metrics, breast-anatomy related metrics, imaging acquisition parameters, and DICOM metadata parameters may not be shown.

The image quality data also includes an overall image quality metric 437 which is an image quality index for all of the images in the image study. This image quality metric 437 may be determined as explained previously herein and described in further detail in PCT application publication WO 2020/102914. In this example, the image quality metric has been stratified across an ordinal scale including the values 1, 2, 3 and 4 with the value 4 indicating the lowest overall image quality and the value 1 indicating the highest overall image quality. The image quality metric 437 provides a quick visual indication on the quality of the images in the image study.

For this example embodiment, in addition to the image quality data, data for the additional measures include breast density data, which is shown in a subwindow 438, cancer risk data which is shown in a subwindow 440, and priority score data (i.e., a priority score) which is shown in a subwindow 442. In other embodiments, these additional measures may not be displayed or only one or two of the additional measures may be displayed.

The breast density data shown in this example embodiment of the GUI 430 includes various information on the breast density of each of the patient's breasts that are shown in the image study. In this example, the breast density data includes a percentage breast density for the right and left breasts which is on a normalized scale of 0 to 100 based on a representative population with higher values indicating higher breast density, which may be associated with a higher cancer risk. The breast density data may include an overall breast density value, representative of the density of both breasts, which in this example is 6%. In addition, in this example, the breast density data includes an index value for the combined overall density of the left and right breasts that is on an indexed scale of A, B, C and D with A being the lowest breast density and D being the highest breast density relative to the representative population for the patient for whom the imaging study was performed. In this example, the breast density index value is an A which is shown as a large letter and this category is also highlighted or shaded in the indexed scale. Another index scale can be used in other embodiments. The normalized numeric breast density score and the indexed A, B, C, D density score is derived from all images in the image study. For example, the left and right numbers can be the average percent density (0-100%) for all left breast and all right breast images, respectively, in the image study. The overall breast density is meant to provide an image study level summary statistic (score) that the IP or other reviewer can see at a quick glance. In other embodiments, the breast density data may include one or two of the indices/scores that are shown in FIG. 4B. The breast density data can be determined after an image study is acquired.

The cancer risk data shown in this example embodiment of the GUI 430 includes a risk score (e.g., 22) which is on a scale determined from data from a large sample or population of breast screening eligible women. An indication of the scale (e.g., 0 to 100) is shown in the subwindow 440 as well as a stratification/classification of risk categories on the scale with lower numbers indicated a standard risk (i.e., lower risk) and higher numbers indicating a priority risk (i.e., higher risk). The risk categories can be based on a cancer risk threshold determined from optimally separating women with breast cancer from those with no breast cancer based on this measure. In some cases, the cancer risk threshold can be user adjustable. The category to which the cancer risk score belongs is also highlighted/shaded in the indexed scale. The cancer risk data can also include a message describing the meaning of the risk levels. In this example, the message states “Patients in the ‘PRIORITY’ category may be considered for supplemental imaging or additional risk assessment”. In other embodiments, the cancer risk data may include just a score or just the risk category.

The priority score data shown in this example embodiment of the GUI 430 includes an indexed scale of values for the priority score that goes from P8 (a low priority score) to P1 (a high priority score). In this example, the priority score is determined as the index value P7, which is not shaded in the indexed scale of P8 to P1. Also, to make it easier for an IP or other reviewer to understand the priority score, the terms “Lower Priority” and “Higher Priority” are placed at opposite ends of the indexed scale. Along with the priority score, a text message may also be displayed. In this example, the message reads “Based on the risk, density and quality assessments associated with this study, the patient has a priority score of P7”.

An example of a technique that can be used to determine the priority score is shown in FIG. 4C in which the cancer risk, breast density and image quality is combined to determine the priority level according to a decision tree structure 450. The cancer risk level is an important part of the priority score, so it is considered at the first level 452 of the tree structure 450 and is stratified between a standard risk score and a priority risk score based on comparing the priority score value to a priority score threshold. The breast density score is at the second level 454 of the tree structure 450 and is stratified between being high density or low density based on comparing the breast density value to a breast density threshold. The image quality is at a third level 456 of the tree structure and is stratified between high quality and poor quality based on comparing an overall image quality value for the image study, such as the image quality metric shown in FIG. 4B for example, to an image quality threshold. The cancer risk threshold, breast density threshold and image quality threshold can be determined by assessing cancer risk values, breast density values and image quality values for a large sample or population of breast screening eligible women with composed of those with breast cancer and those with no breast cancer and selecting each of the thresholds such that together they result in a pattern whereby the increasingly higher priority score (i.e. closer to P1) identifies groups of women with increasingly higher cancer rates.

An example that shows how the decision tree structure 450 functions to provide a priority index value assumes that the cancer risk index is standard risk, the breast density index is high density and the image quality index is poor quality. In this case, the priority index value is P5, which is a borderline score between low risk and high risk on the priority index scale. The priority index score may be used as an indicator of image study complexity for workload management through worklist filtering/sorting (e.g., for the worklist services software component 106). For example, the priority index score can allow an organization to automatically assign more complex image studies (e.g., image studies having a priority score indicating a higher risk) to radiologists with more experience by updating their image study worklist file with the image study ID of the complex image studies, or to move the most complex cases (e.g., priority index scores P1, P2 and P3) to the top of the image study worklist file for certain radiologists.

Referring back to FIG. 4A, in one example embodiment the method 400 can include acts 410 and 412 where an image quality recommendation is generated, and the image quality recommendation is displayed, respectively, in the GUI. For example, referring to FIG. 4B, a text indicator 446 can be displayed when the method 400 recommends VQR. The VQR recommendation can be determined by comparing the image quality metric to an image quality threshold such as 2, for example. Therefore, when the image quality metric is higher than 2 (indicating poorer than acceptable image quality) then VQR is recommended and when the image quality metric is 2 or lower (indicating greater than acceptable image quality) then VQR is not recommended. The VQR recommendation may be implemented by the recommendation module 226. The image quality threshold may be set to a default value and/or it may be user specified.

If the IP or reviewer agrees, then they can select button 448 to send the image study to VQR. If the IP or reviewer selects the button 448 then the method 400 receives the reviewer's command to send the image study to VQR at act 414 and takes the corresponding action. For example, in this case, the MIQA application 220 can electronically document that the image study is to be sent to VQR and update the database 230 to reflect this as well as communicate with the worklist services software 106 to add the image study to a VQR worklist and/or communicate with the RIS and reporting services software 108 to include the IP's assessment that the image study be sent to VQR.

In another example embodiment, act 412 can also include the generation and display of a pop-up message, such as window 460 as shown in FIG. 4D. The window 460 can be displayed to show a first message 462 that the MIQA application 220 recommends that the image study be sent to VQR and a second message 464 providing the rationale for the VQR recommendation. In this case, the rationale for the recommendation is that the image quality metric is 4, which indicates poor quality.

For either of the embodiments shown in FIGS. 4C and 4D, if the IP agrees, a command is received to send the image quality to VQR, then the method 400 can include updating the GUI 430 as shown in FIG. 4E with the text indicator 449 to indicate that the MIQA application 220 has taken action to electronically record the IP's decision and notify the appropriate software of the medical imaging system 100 as described above.

Alternatively, if the method 400 does not recommend sending the image study to VQR then no messages to this effect are displayed as shown for GUI 470. Rather, as shown in FIG. 4F, the method 400 can provide the IP with the ability to initiate VQR by displaying the text indicator 472 (e.g., “Initiate VQR”) and displaying the input button 474. If the IP decides to send the image study for VQR and selects the button 474, then the method 400 receives the IP's command at act 414 and takes the corresponding action. For example, the MIQA application 220 can take the actions that were previously described and the method 400 can display the GUI 430 shown in FIG. 4E to confirm that the image study has been selected for VQR.

In at least one embodiment, the GUI 470 also includes an I/O field 433 that displays text indicating whether any comments have been made by a reviewer regarding the quality of the image study. If a reviewer wishes to enter comments, then they may click on/select the I/O field 433. When the method 400 receives an indication that the reviewer clicked on (i.e., selects) the I/O field 433, which may be referred to as an image quality text feedback command, the method 400 involves opening a subwindow with a free-text box (both not shown) where the reviewer can enter/type in comments. The comments may include previous entries by the MIT such as, but not limited to, remarks about the patient habitus (i.e., the patient's physique), and remarks about other factors that might make the patient particularly difficult to position for a mammogram. These comments from the MIT can provide some insights to the reviewer as to the possible reasons for certain image positioning errors in the image study. The reviewer can make any comments they wish to make which may be remarks that label the image study as being a good quality image study which can later be provided to quality inspectors in a report when they are doing a mammography facility inspection, and/or other labels that can be used to indicate other potentially useful information about the patient or about how to manage the image study.

In an alternative embodiment, rather than generate a text box in which the reviewer is free to enter any text they want, when the user selects the I/O field 433, then a drop-down menu can be displayed which provides standardized feedback options from which the reviewer can make one or more selections. The standardized feedback options can be a list of feedback items where one or more of the feedback items can be similar to the feedback discussed above for the “free-text feedback entry” embodiment. By providing standardized feedback items for the reviewer to choose from, this allows for the feedback to be more consistent, and faster for the reviewer to enter compared to typing the feedback in a free-text box.

In at least one embodiment, as shown for the GUIs 430 and 470 in FIGS. 4B, and 4D-4F, the IP or other reviewer has the ability to edit the image quality data that is displayed by selecting an edit function by selecting the edit button 444. The IP may wish to do this if they strongly disagree with the displayed image quality data and if the IP has been given clearance to modify the image quality data. For example, the error and pass symbols that are displayed on the GUI 430 may be editable when the reviewer selects the edit button whereby when the reviewer then clicks or selects one or more of the image quality symbols, the processor receives this user selection, and flips the image quality symbol so that if the displayed image quality symbol was originally an error symbol then it is changed to and displayed as a pass symbol or if the displayed image quality symbol was originally a pass symbol then it is changed to and displayed as an error symbol. After the IP has finished editing the displayed image quality data, the IP can select the edit button 444 again to disable editing. At that point the MIQA application 220 can electronically record the edited image quality data in one of the databases 230 in order to update the image quality data for the image study being review. The edited image quality data may be used for training the algorithms that are used for generating the image quality data. The edited image quality data may also be used as the basis for any VQR for this image study, and for any reports or computations that incorporate image quality results from this image study. The edited quality data may also be incorporated into any visualizations of image quality.

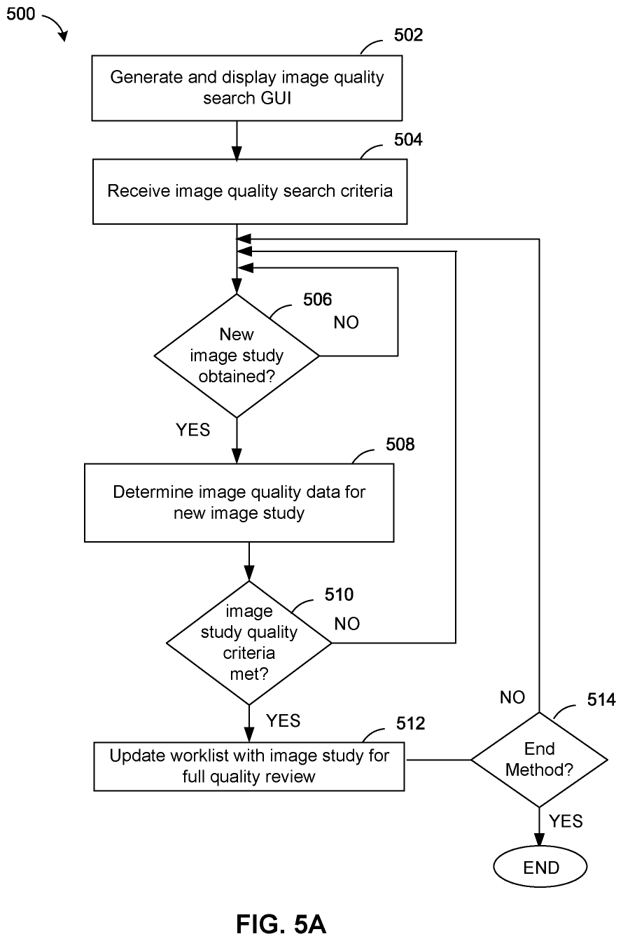

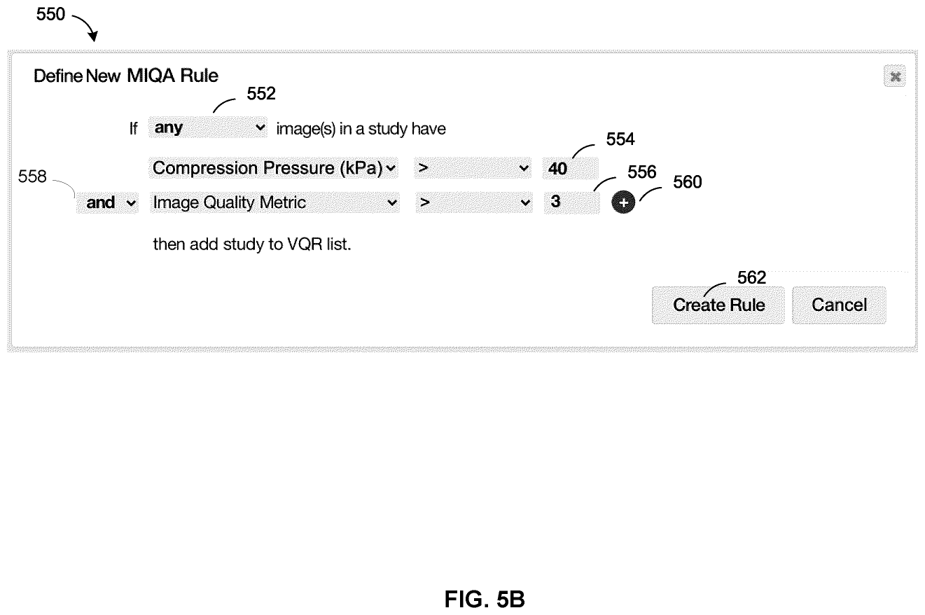

Referring now to FIG. 5A, shown therein is a flow chart of an example embodiment of a method 500 for automatically identifying image studies for image quality review (e.g., added to a VQR list). The method 500 may be performed by a processor of the processor unit 202 in FIG. 2. Method 500 allows for a user, such as a QC manager or other user for example, to automatically identify image studies to be added to the VQR list for a visual quality assessment, based on predetermined VQR criteria. This may be done because the QC manager or other user believes that the image studies that are captured by the VQR criteria are at a higher risk of having poor image quality. Method 500 is advantageous in that it allows for image studies to be automatically identified for VQR without having to rely on input from the IP. For example, the VQR criteria can be defined such that image studies with known combinations of VQR criteria are identified, that the LIP may want added to the VQR list, but the IP's may not be aware of. Alternatively, method 500 allows for the identification of image studies requiring VQR to be done in a more repeatable standardized fashion thereby reducing bias and subjectivity.

Method 500 it typically used to assess all image studies as they are acquired. Alternatively, in another embodiment, method 500 can be used to assess all previously obtained image studies to detect if any review is necessary.

Method 500 begins at act 502 where an image quality search UI is generated and displayed. An example is GUI 550 which is shown in FIG. 5B. The GUI 550 provides an interface for a user to define the VQR criteria that are used to automatically identify image studies that require VQR. In this example, the GUI 500 includes an input option, such as a drop-down menu 552, for allowing the user to select one or more of views (e.g., CC view, etc.) or a portion of the views in an image study to use to make the VQR determination. As an example, the options of the drop-down menu 552 may be: any images, all images or no images. The GUI 550 displays a series of input options, such as drop-down menus and text boxes that are arranged in a row format to allow the user to specify the VQR criteria that are applied to certain images of the new image study by defining and combining, via at least one logical operator 558, at least two VQR criteria where each VQR criteria involves an image parameter feature that is selected by the user and then compared to a threshold value that is selected by the user to perform the automated searching of image studies that have poor image quality.

In this example, the first VQR criteria 554 defines that the image parameter feature “Compression Pressure” is greater than 40 kPa and the second VQR criteria defines that the Image quality metric is greater than 3. The logical operator 558 is set to the logical “and” operator, which means that when both the first and second VQR criteria are true for any image in an image study then that image study is flagged for addition to a VQR list. A software program from the medical imaging system 100, such as the reporting services software 110 or the RIS and worklist services software 106 can receive an electronic message indicating that the image study is to undergo VQR. The user may add further VQR criteria by selecting the add button 560. Once the user is satisfied with the automated VQR criteria the user can select the create rule 562. The created VQR criteria can then be stored, such as in the data files 232, or another file structure.

It should be noted that many other types of parameters may be used in the VQR criteria including, for example, any combination of: (a) image quality metric score, (b) breast density score, (c) cancer risk score, (d) priority score, (e) positioning metrics including one or more of: PNL Difference >10 mm, CC exaggeration, portion cut-off, skin folds, posterior tissue missing, inadequate pec muscle length, pec muscle concave, IMF missing, IMF inadequate, MLO sagging, and/or MLO too high on IR, (f) image features including one or more of sharpness, contrast, exposure, noise, artifacts, nipple in profile and/or MLO angle, (g) imaging acquisition parameters including one or more of radiation dose, compression pressure and/or compression force, (h) breast related metrics including breast volume and/or breast area and (i) DICOM metadata parameters including one or more of age, sex, weight, and/or imaging machine ID. In other embodiments there may be other parameters that can be used in the VQR criteria.

Referring back to FIG. 5A, once the VQR criteria for the image quality search is defined, the method 500 involves receiving at least one VQR criterion from the or a user defined combination, via at least one logical operator, of at least two VQR criteria where each VQR criteria involves an image parameter feature that is selected by the user, a comparison operator (e.g., >, <, =, etc.) and a threshold value that is selected by the user and storing these user selections for the VQR criteria at act 504.

The method 500 then proceeds to act 506 where it monitors the activity over the network 110 to determine when a new image study has been obtained/acquired by an MIT. Once this occurs, the method 500 proceeds to act 508 where the method 500 determines the image quality data, which may be done using the image quality analysis module 222. At act 510, the image quality data are used to determine when the VQR criteria are met. If VQR criteria is satisfied, then the method 500 proceeds to act 512 where the method 500 may update the VQR worklist with the image study ID for the new image study. If the determination at act 510 is not true, then the method 500 moves to act 506 where it monitors the network 110 for the acquisition of the next image study. After act 512, the method 500 proceeds to act 514 where it is determined whether the method 500 should continue to monitor the network 110 for the acquisition of a new image study in which case the method 500 proceeds to act 506. Alternatively, at act 514 it may be determined that no further image studies will be analyzed to determine whether they should be sent for VQR in which case the method 500 ends.

In at least one embodiment, the method 500 is modified to keep track when a number of image studies that were acquired by a given MIT that meet the VQR criteria over a certain time period, such as, but not limited to a period of one, two or three weeks; one, two, or three months; or some other time period. This may enable for VQR review for the given MIT to be electronically noted and possibly subsequent remedial action may be determined based on the nature of the image quality issues, which may be based on a pattern of observed image quality problems, e.g., repeated instances of a specific type of error are observed.

In at least one embodiment, the method 500 is modified to keep track when a number of image studies that were acquired by the same MIT are compared to VQR criteria to determine whether an overall image study quality, across all of the image studies, for the MIT drops below the MIT's typical image quality levels over a certain time period. This may trigger a review to determine whether there are any anomalies in the quality assessment or the acquisition environment.

In at least one embodiment, the VQR can be electronically documented to be performed on one or more of the image studies that were part of the set of the image studies that triggered the alert. For example, if the VQR was triggered by having at least 10 image studies with an MLO sagging problem, then it may be desired to review a sample of at least 3 of those 10 image studies but not necessarily all 10 image studies as this allows for this review to be more feasible since it requires less review time to review 3 rather than 10 image studies.

Either of the above quality reviews are difficult to do without an image quality review system that can automatically comprehensively assess image quality for all image studies. Triggering a VQR based on a single image study may be valuable for a severe case (e.g., where the image quality is very poor), but it may also be valuable to trigger a VQR for problematic trends or patterns determined based on the image quality of several image studies.

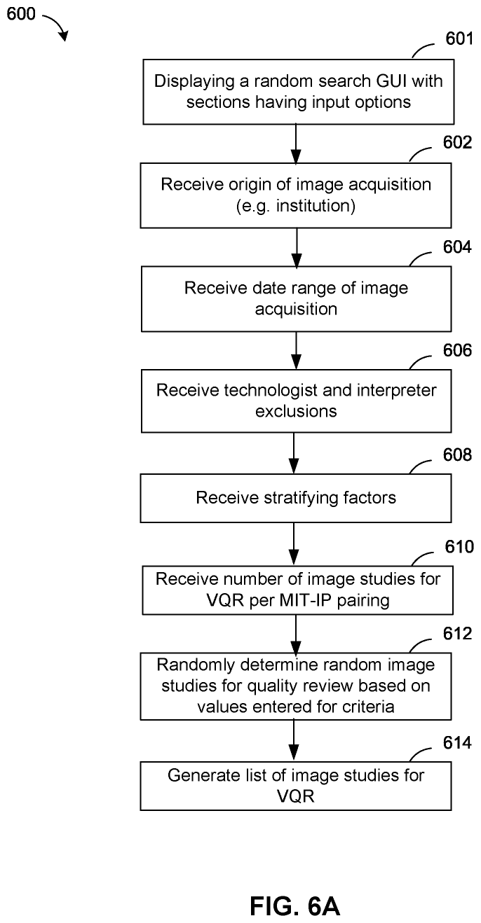

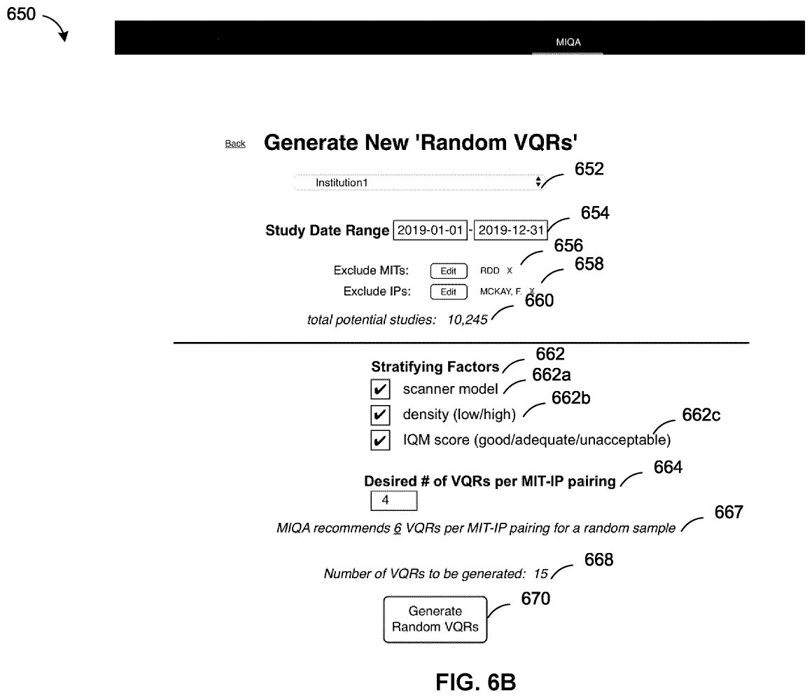

Referring now to FIG. 6A, shown therein is a flow chart of an example embodiment of a method 600 for randomly generating a list of image studies for image quality review (e.g., VQR). The method 600 may be performed by a processor of the processor unit 202 in FIG. 2. The method 600 cannot be performed manually as it is not possible to select a true random set of image studies from very large sets of image studies on a manual basis.

Random sampling of image studies for VQR is important since it enables reduction of potential biases in studies selected for VQR that may be introduced by one or more factors such as scanner model, breast density, and image quality metric, amongst a plurality of other factors. Random sampling of image studies from blocks, which are defined by combinations of these factors within unique MIT-IP pairing pairs, to reduce the chance that the image studies that are randomly selected are unbalanced on any of these factors and achieves better generalization of results from the VQR process. An added aspect of the random sampling process is to specify the desired number of VQRs per MIT-IP pairing, which is the pairing of an MIT with an IP indicating the MIT that acquired the image study and the IP that reviewed the quality of the image study. The reason for this is so that the actual random sample of image studies that are determined is a manageable size for the reviewer to feasibly review.

To ensure true randomization a random seed may be generated by software applications that include, but are not limited to, database software and functions provided by programming languages that are used to implement the MIQA application 220 so that the studies from within the blocks are randomly selected. The random seed can be set in a plurality of ways including, but not limited to, using the date and time setting from the clock of the local computer system on which the MIQA application 220 is installed at the time that the set of image studies are identified for VQR, for example. The random sampling may be triggered through an interface (e.g., as shown in FIG. 6B) or software (such as a web application) that are displayed to the user. Random sampling can also be algorithmically performed on demand by specifying blocks and the desired number of VQRs per MIT-IP pairing as described above. In at least one embodiment, the random sampling of image studies may also allow for the exclusion of certain MITs or IPs who may no longer be active.

In the example embodiment shown in FIG. 6A, the method 600 displays a random search GUI to a user to select criteria for generating the random list of image studies for VQR. An example of a GUI that may be used is GUI 650 shown in FIG. 6B. Accordingly, the method 600 generally includes step 601 which involves displaying the GUI 650 with sections having input options to allow the user to specify search criteria. For example, step 601 may include displaying the GUI 650 with a first section with at least one input option to allow the user to select one or more initial search criteria; displaying the GUI 650 with a second section with at least one second input option for allowing the user to select one or more stratifying factors; and displaying the GUI 650 with a third section with a third input option to allow the user to specify a desired number of images studies for VQR for a given pairing of a MIT and IP in a random sample. The method 650 further generally includes steps 602 to 610 for receiving the user selections for the input options; step 612 for generating a random list of image studies for VQR using the user selections; and step 614 for storing the random list of the image studies for VQR.

In at least one embodiment, the method 600 may optionally include displaying a recommended number of image studies for VQR fora given pairing of a MIT and IP in the random sample in order to allow for a more effective review (e.g., provide an adequate sample size for the number of image studies for VQR) of the given pair of MIT and IP from an image quality perspective. This allows the user to reconsider and change the desired number of images studies for VQR for a given pairing of a MIT and IP. The recommended number of image studies for each MTI-IP pair is determined by finding the number of images studies that will result in a more balanced box size.

Additionally, or in alternative thereto, in at least one embodiment, the method 600 may optionally include displaying the number of image studies for VQR based on the user selections. This allows the user to reconsider the user selection for at least one of the input options so that the number of image studies for VQR is an acceptable number that can be reviewed given the resources that are available for performing the review.

The GUI 650 includes presenting at least one first input option by providing an institution drop down menu 652 where the user can select the institution from which the image studies are to be randomly sampled. Optionally, the institution drop-down menu 652 can also specify a particular department as an institution may have many departments or it might be a particular institution within a region (i.e., state, province or nationwide). The selection by the user is received at act 602 of method 600. Optionally, the GUI 650 also includes date range selection input boxes 654 as part of at least one first input option that the user can use to specify that the image studies are to be randomly selected from image studies acquired between the starting and ending dates specified by the user in the input boxes 654. The starting and ending dates for the date range of image study acquisition are received by the method 600 at act 604.

Optionally, the GUI 650 can also include text boxes 656 and 658 as part of at least one first input option where the user can specify that image studies acquired by one or more MIT selections and/or interpreted by one or more IP selections, respectively, are not to be part of the random selection of image studies. The user can repeatedly select the respective edit buttons to keep adding the names of the MITs and/or IPs that are to be excluded for the study. These exclusions may be made since some MITs and IPs are no longer with the institution are do not need to be assessed. Alternatively, in another embodiment, the text boxes 656 and 658 may be used by the user to provide one or more IP selections and/or one or more MIT selections to include in the random selection of image studies.

At act 606 the exclusion data for the MITs and IPs for which image studies are excluded in generating the random list of image studies is received by the method 600. At this point, the method 600 can determine a potential number of image studies, based on the criteria provided thus far by the user in the first input options, from which the image studies for VQR can be randomly sampled and may display this number at text message 660 of the GUI 650.

The GUI 650 also provides one or more second input options 662 for the user to select from in order to stratify the random selection of image studies to reduce bias in the selected random sample, as described above. For example, the GUI 650 can provide one or more of input checkboxes 662a, 662b, and/or 662c that the user can select to stratify based on scanner model, breast density value, and/or image quality metric score, respectively. In other embodiments, there can be various combinations of the input checkboxes 662a, 662b and 662c. The selection by the user is received at act 608 of the method 600. In at least one embodiment, the GUI 650 may include further input options to allow for further stratification. For example, the GUI 650 may include for input options to allow the user to specify large breasts versus small breasts, a number of image studies for large breasts and/or a number of image studies for small breasts to include in the random sampling.

The GUI 650 may also include an input text box 664 for allowing the user to specify the number of image studies that are randomly selected for VQR from the blocks within the unique MIT-IP pairings. This allows the user to obtain a smaller randomly selected subset of the image studies that were randomly selected for VQR within each unique MIT-IP pairing so that the MIQA application 220 can generate a number of VQRs that the reviewers can feasibly review. The selection by the user is received at act 610 of the method 600.

The GUI 650 may also include a text message 667 in which the user is provided with a recommendation of the number of image studies per MIT-IP pairing that should be selected by the user to achieve a balanced random sample with at least one image study selected from each block. The text message 667 may be generated and displayed to the user at act 612 of the method 600. At this point the user may go back and change the entry at input text box 664 if they think that the recommendation shown in text message 667 is acceptable.

The GUI 650 may also include a text message 668 which indicates the total number of image studies that will be selected for VQR based on all of the inputs provided by the user thus far. The text message 668 may be generated and displayed to the user at act 612 of the method 600. If the user thinks that this number of image studies that will be selected is not acceptable then they can change one or more of the inputs that they have provided until the text message 668 indicates a number of VQRs that the user believes is acceptable. In this context, “acceptable” means if the number of VQRs is very high and the resources required to review all the image studies is very time/resource consuming, then the user may decide to settle on a lower number that they can actually manage to review. Alternatively, if the number is very low the user may want more image studies reviewed to provide sufficient data to confidently assess quality. Alternatively, or in addition thereto, regional or organizational accreditation/quality standards may be used to indicate the minimum or desired number of VQRs (i.e., the number of image studies to go under VQR) that are required.

The GUI 650 includes input button 670 which the user can select when they are satisfied with all of the inputs they have provided and the number of random image studies that will be selected for VQR. At this point, at acts 612 and 614 the method 600 receives the command from the user, proceeds to randomly determine the image studies for VQR based on the input values entered for the various criteria shown in GUI 650 and then adds the randomly selected image studies to a VQR worklist. This VQR worklist may be conveyed to the worklist services software 106. In some embodiments, the VQR worklist can be saved at the database 230 of the MIQA server 104.

TABLE 4
EXAMPLE OF RANDOM IMAGE STUDY SELECTION FOR VQR

| | | | | S = 3 | D = 2 | Q = 3 | |
|---|---|---|---|---|---|---|---|
| P = I*J | B = S*D*Q | I = 2 | J = 2 | s = 1 to S | d = 1 to D | q = 1 to Q | |
| p = 1 to P | b = 1 to B | i = 1 to I | j = 1 to J | Scanner | Density | IQM | |
| Pair(p) | Block(b) | MIT (i) | IP (j) | Model | H/L | (G/A/U) | n(b) |
| Pair(1) | Block(1) | 1 | 1 | 1 | H | G | n(1) |
| Pair(1) | Block(2) | 1 | 1 | 1 | H | A | n(2) |
| Pair(1) | Block(3) | 1 | 1 | 1 | H | U | n(3) |
| Pair(1) | Block(4) | 1 | 1 | 1 | L | G | n(4) |
| Pair(1) | Block(5) | 1 | 1 | 1 | L | A | n(5) |
| Pair(1) | Block(6) | 1 | 1 | 1 | L | U | n(6) |
| Pair(1) | Block(7) | 1 | 1 | 2 | H | G | n(7) |
| Pair(1) | Block(8) | 1 | 1 | 2 | H | A | n(8) |
| Pair(1) | Block(9) | 1 | 1 | 2 | H | U | n(9) |
| Pair(1) | Block(10) | 1 | 1 | 2 | L | G | n(10) |
| Pair(1) | Block(11) | 1 | 1 | 2 | L | A | n(11) |
| Pair(1) | Block(12) | 1 | 1 | 2 | L | U | n(12) |
| Pair(1) | Block(13) | 1 | 1 | 3 | H | G | n(13) |
| Pair(1) | Block(14) | 1 | 1 | 3 | H | A | n(14) |
| Pair(1) | Block(15) | 1 | 1 | 3 | H | U | n(15) |
| Pair(1) | Block(16) | 1 | 1 | 3 | L | G | n(16) |
| Pair(1) | Block(17) | 1 | 1 | 3 | L | A | n(17) |
| Pair(1) | Block(18) | 1 | 1 | 3 | L | U | n(18) |
| Pair(2) | Block(1) | 1 | 2 | 1 | H | G | n(1) |
| Pair(2) | Block(2) | 1 | 2 | 1 | H | A | n(2) |
| Pair(2) | Block(3) | 1 | 2 | 1 | H | U | n(3) |
| Pair(2) | Block(4) | 1 | 2 | 1 | L | G | n(4) |
| Pair(2) | Block(5) | 1 | 2 | 1 | L | A | n(5) |
| Pair(2) | Block(6) | 1 | 2 | 1 | L | U | n(6) |
| Pair(2) | Block(7) | 1 | 2 | 2 | H | G | n(7) |
| Pair(2) | Block(8) | 1 | 2 | 2 | H | A | n(8) |
| Pair(2) | Block(9) | 1 | 2 | 2 | H | U | n(9) |
| Pair(2) | Block(10) | 1 | 2 | 2 | L | G | n(10) |
| Pair(2) | Block(11) | 1 | 2 | 2 | L | A | n(11) |
| Pair(2) | Block(12) | 1 | 2 | 2 | L | U | n(12) |
| Pair(2) | Block(13) | 1 | 2 | 3 | H | G | n(13) |
| Pair(2) | Block(14) | 1 | 2 | 3 | H | A | n(14) |
| Pair(2) | Block(15) | 1 | 2 | 3 | H | U | n(15) |
| Pair(2) | Block(16) | 1 | 2 | 3 | L | G | n(16) |
| Pair(2) | Block(17) | 1 | 2 | 3 | L | A | n(17) |
| Pair(2) | Block(18) | 1 | 2 | 3 | L | U | n(18) |

An example of how the image studies can be randomly selected is now provided. It should be noted that there may be other techniques that may be used for randomly generating the list of image studies for VQR and this is a non-limiting example.

Consider generating a random sample of M image studies for VQR for each MIT-IP pairing Pair(p) (i.e., MIT-IP pairing) where p=1 to P and P=I*J, where P is the number of unique MIT-IP pairings, I is the number of MITs, J is the number of IPs, S is the number of scanner vendor models (e.g., S=3; V1M1/V1M2/V2M1 where the first two characters indicate the vendor and the last two indicate the imaging machine model), D is the number of breast density categories (e.g., D=2; high/low) and Q is the number of IQM score categories (e.g., Q=3; Good/Adequate/Unacceptable). From input text box 664 of the GUI 650, the user specifies the desired number of m VQRs (image studies) to randomly select from each MIT-IP pairing Pair(p) from the database 230 used by the MIQA application 220.

Within each MIT-IP pairing Pair(p), the method 600 identifies each Block(b) which is defined by a unique combination of Scanner vendor model, Breast Density value, and IQM Score, where b=1 to B, and B=S*D*Q is the number of blocks within each MIT-IP pairing Pair(p) (e.g., 3*2*3=18). The number of image studies in each Block(b) is n(b). Within each MIT-IP pairing Pair(p), the method 600 randomly identifies image studies from the n(b) image studies in each Block B(b) fora total of M (where M=B) image studies per MIT-IP pairing, and recommends a total set of M*P image studies for a representative random sample of image studies for VQR. An example of these calculations is shown in Table 4.

In at least one embodiment, the method 600 may also provide for a user-specified number of M (where M is not equal to B) image studies from input text box 664 to be randomly selected from the set of B image studies per MIT-IP pairing for VQR.

Referring now to FIG. 7A, shown therein is an example embodiment of a method 700 for investigating and recording operator performance during an initial or subsequent VQR of an image study. The method 700 is beneficial in that it provides digitization and visualization of a more thorough image quality assessment for an image study performed by a particular MIT and allows for the assessment to be more standardized. The method 700 also allows the reviewer to perform a thorough image quality assessment since more image quality data is provided and the reviewer has the ability to provide a greater amount of feedback in their assessment. The method 700 may be performed by a processor of the processor unit 202 in FIG. 2.

The method 700 begins at act 702 where a request for VQR of an image study is received along with the ID for the image study. For example, this may be done by sending an electronic message to a user who is a reviewer, such as an IP or another reviewer, where the electronic message includes the request for the reviewer to perform VQR of a first image study which may be identified by an image study ID. The electronic message may be in the form of an email or an updated worklist file. The method 700 proceeds to act 704 where the image quality data that corresponds to the first image study is retrieved from the database 230 by searching on the image quality data associated with the image study ID. At act 706, one or more image quality GUIs are shown along that include at least a portion of the image quality data. The image quality data can be provided for a number of image quality categories 722 which may be related to a combination of breast positioning, image acquisition and/or DICOM metadata. Only one of the image quality categories is shown with reference numerals for ease of illustration. A summary of the image quality data can be provided in a first image quality GUI such as GUI 720 which is shown in FIG. 7B.

In this example embodiment, the summary of the image quality data may include one or more image quality categories, an index of possible scores for the image quality categories and a score value for the image quality categories. In the example shown in FIG. 7B, the image quality categories that are displayed are for positioning, compression, exam ID, artifacts, exposure, contrast, sharpness and noise. In other embodiments, there might be a different combination of image quality categories that are shown including image quality categories that are not shown in FIG. 7B. For each image quality category shown in the GUI 720, an index of possible scores is given, which in this example are scores A, B, C and D, along with a short description of what the score means. In this example, a score of A means textbook perfect image quality, a score of B means good image quality, a score of C means acceptable image quality but might be better and a score of D means inacceptable image quality and the image study should be repeated. The particular score value for each image quality category is highlighted or outlined. This allows the reviewer to quickly scan the image quality summary and get a good sense of the overall image quality such as, for example, when more scores are displayed along the left-hand side rather than the right-hand side of the list of possible scores then this provides a quick visual indication that the image quality of the image study is better and not worse than average, for example. In other embodiments, other indices or scores can be used to represent different levels of image quality and the method 700 is not limited to using the scores A, B, C and D.

The method 700 then includes determining whether the reviewer wishes to examine one or more of the image quality categories in greater detail and possibly receive image quality feedback data from the reviewer for the examined image quality categories. For example, when the reviewer selects any of the image quality categories 722, such as by clicking on the score for a particular image quality category, the method 700 includes receiving the selected image quality category and then displaying a subsequent image quality GUI with more detailed image quality data for the selected image quality category. Examples of these GUIs are shown in FIGS. 7G-7N.

Some of the more detailed image quality data that is displayed is typically prepopulated based on the image quality data that has been retrieved for the image study undergoing VQR. However, in at least one embodiment, some of the image quality data for a particular image quality category may be blank/empty so that the reviewer can enter their assessment of the image quality for that particular image quality category. The user supplied image quality data can then be stored in a database or file along with the other image quality data that corresponds to the image study undergoing VQR.

In addition, or in at least one other embodiment, the reviewer can review the more detailed image quality data that is automatically prepopulated and displayed and then provide feedback by making changes to this prepopulated image data that the reviewer does not agree with. For example, the reviewer feedback can be from the reviewer picking a different score (e.g., if A was selected, they may decide B was more appropriate and pick it) when the reviewer is viewing a GUI for a particular image quality category.

In any of these embodiments, any image quality feedback data from the user that is received by the processor is then stored in the database 230. The image quality feedback data may also be communicated to other elements of the medical image system 100 such as the reporting services software 108, for example.

Furthermore, in at least one embodiment, the image quality feedback data provided by the user may be used for future Image Quality Parameter modelling. The image quality feedback data may include expert assessment of a broad range of IQPs that span positioning errors and/or non-positioning errors such as, but not limited to, one or more of poor compression, presence of specific artifact types such as hair or others, underexposure, overexposure, high or low contrast, poor sharpness and noise patterns, for example. In at least one embodiment, the methods described previously herein and in PCT application publication WO 2020/102914 may be used for training to obtain a more effective classifier to identify images that contain the artifacts and conditions whose presence is indicated by the image quality feedback data collected through the VQR process.

Accordingly, act 708 of method 700 involves receiving and storing image quality feedback data from the reviewer which may continue until the reviewer completes the VQR of the image study. For example, although not shown, the GUI 720 can include an input button that the reviewer selects when they are finished examining and possibly editing any desired image quality parameter features 722.

For example, referring now to FIG. 7G, shown therein is image quality GUI 750, which provides more detailed image quality data for the “positioning” image quality category. The GUI 750 displays the image quality category (i.e., positioning) and optionally includes a list of the possible scores 751 and the selected score 752 similar to what was shown in the quality summary GUI 720. The GUI 750 also includes a table 753 with more granular image quality detail for a plurality of image quality parameter features that relate to the image quality parameter, e.g., the “positioning” image quality category in this case, for one or more images of the image study. The first column lists the different image quality parameter features, which in this example are PNL difference >10 mm (CC v MLO), Inadequate IMF, MLO Sagging, Posterior Tissue Missing, Portion Cutoff, Skin Folds, Inadequate Pectoralis, CC Exaggeration and Other Body parts over breast. In other embodiments, other image quality parameter features can be shown here, or a different combination of image quality parameter features can be shown here. The table 753 includes columns for at least one of the images in the image study that is undergoing VQR and input fields for allowing the reviewer to enter feedback on or more images of the image study. For example, the input fields may be a checkbox that is placed in each row for a particular image where the image quality is placed in each row for a particular image where the image quality parameter feature is applicable, as some are only relevant for CC or MLO images. These image quality parameter features from the displayed images of the image study can then be assessed by the reviewer by entering checkmarks where the reviewer thinks that those particular aspects were present in the image study. In an alternative embodiment, these checkboxes may be prepopulated with checkmarks and displayed based on the automated image quality data results that has been retrieved for the image study undergoing VQR. These prepopulated checkmarks may be modified by the reviewer.

In at least one embodiment, as shown in FIG. 7G, for every row that receives a checkmark from the reviewer, the table 753 may also provide a means for the reviewer to record, based on their review, whether the deficiency was the result of technologist technique or the patient's ability to cooperate by selecting the radio button in either of these two columns.

In at least one embodiment, as shown in FIG. 7G, the table 753 may also include a final column that is labelled “Other” which comprises text boxes to allow the reviewer to enter feedback on each of the image quality parameter features that are being assessed that are related to positioning. Although not shown in FIG. 7G, a “save” input button can be added so that all of the entries made by the reviewer are received by the processor and saved to the database 230.

Referring now to FIG. 7H, shown therein is image quality GUI 755 which provides more detailed image quality data for the “compression” image quality parameter category. The GUI 755 displays the image quality category (i.e., compression) and optionally includes a list of the possible scores 756 and the selected score 757 similar to what was shown in the image quality summary GUI 720. The GUI 755 also includes a table 758 with more granular image quality detail for a plurality of image quality parameter features related to the “compression” image quality category. The first column lists the different image quality parameter features, which in this example are Poor separation of breast tissue and Uneven exposure. In other embodiments, other image quality parameter features can be shown here. The table 758 includes columns for at least one of the images in the image study that is undergoing VQR with checkboxes (i.e., input fields for allowing the reviewer to enter feedback for one or more images of the image study) as was described for GUI 750. In at least one embodiment, the table 758 may also include columns for other aspects to consider for compression including one or more of “patient motion”, “Under compression by Tech” and/or “Positioning of compression device by Tech”, for example. These can be assessed by the reviewer by entering checkmarks where the reviewer thinks that those particular aspects were present in the image study. The data entered by the reviewer is received by the processor. As with GUI 750, although not shown in FIG. 7H, a “save” input button can be added so that the processor can save all of the entries made by the reviewer to the database 230.

Referring now to FIG. 7I, shown therein is image quality GUI 760 which provides more detailed image quality data for the “exam ID” image quality parameter category. The GUI 760 displays the image quality category (i.e., exam ID) and optionally includes a list of the possible scores 761 and the selected score 762 similar to what was shown in the image quality summary GUI 720. The GUI 760 also includes a table 763 with more granular image quality detail for a plurality of image quality parameter features related to the “exam ID” image quality category. The first column lists the different image quality parameter features, which in this example are Patient name and additional patient identifier, facility name and location, date of examination, view and laterality, unit identification and technologist identification. In other embodiments, other image quality parameter features can be shown here, or a sub-combination of the image quality parameter features currently shown in FIG. 7I may be displayed in the table 763. The table 763 includes columns for at least one of the images in the image study that is undergoing VQR with checkboxes (input fields for allowing the reviewer to enter feedback for one or more images of the image study) as was described for GUI 750. The reviewer can click the checkboxes where the image quality parameter features were included in each of the views in these columns. In at least one embodiment, the table 763 may also include columns to allow the reviewed to consider and record other aspects for exam ID including “technologist error”, and/or “Missing of non-standard labelling method”, for example. These can be assessed by the reviewer by entering checkmarks where the reviewer thinks that those particular aspects were present in the image study. The data entered by the reviewer is received by the processor. As with GUI 750, although not shown in FIG. 7I, a “save” input button can be added so that the processor can save all of the entries made by the reviewer to the database 230.

Referring now to FIG. 7J, shown therein is image quality GUI 765 which provides more detailed image quality data for the “artifacts” image quality parameter category. The GUI 765 displays the image quality category (i.e., artifacts) and optionally includes a list of the possible scores 766 and the selected score 767 similar to what was shown in the image quality summary GUI 720. The GUI 765 also includes a table 768 with more granular image quality detail for a plurality of image quality parameter features related to the “artifacts” image quality category. The first column lists the different image quality parameter features, which in this example are hair, deodorant, grid related, IR related, detector calibration, foreign objects calibrated into calibration file, uncertain and other. In other embodiments, other image quality parameter features can be shown here, or different combinations of the image quality parameter features currently shown in FIG. 7J may be displayed. The table 768 includes columns for at least one of the images in the image study that is undergoing VQR with checkboxes (input fields for allowing the reviewer to enter feedback for one or more images of the image study) as was described for GUI 750. The reviewer can click the checkboxes where the image quality parameter features were included in each of the views in these columns. The data entered by the reviewer is received by the processor. As with GUI 750, although not shown in FIG. 7J, a “save” input button can be added so that the processor can save all of the entries made by the reviewer to the database 230.

Referring now to FIG. 7K, shown therein is image quality GUI 770 which provides more detailed image quality data for the “exposure” image quality parameter category. The GUI 770 displays the image quality category (i.e., exposure) and optionally includes a list of the possible scores 771 and the selected score 772 similar to what was shown in the image quality summary GUI 720. The GUI 770 also includes a table 773 with more granular image quality detail for a plurality of image quality parameter features related to the “exposure” image quality category. The first column lists the different image quality parameter features, which in this example are widespread underexposure, widespread overexposure, insufficient penetration of dense areas, and too much penetration of lucent areas. In other embodiments, other image quality parameter features can be shown here, or different combinations of the image quality parameter features currently shown in FIG. 7K may be displayed. The table 773 also includes columns for at least one of the images in the image study that is undergoing VQR with checkboxes (input fields for allowing the reviewer to enter feedback for one or more images of the image study) as was described for GUI 750. The reviewer can click the checkboxes where the image quality parameter features were included in each of the views in these columns. In at least one embodiment, the table 773 may also include a column labelled “causes” with text boxes for each of the image quality parameter features. The reviewer can enter text feedback on what they think was the cause of certain issues with the image quality parameter features listed in table 773. The data entered by the reviewer is received by the processor. As with GUI 750, although not shown in FIG. 7K, a “save” input button can be added so that the processor can save all of the entries made by the reviewer to the database 230.

Referring now to FIG. 7L, shown therein is image quality GUI 775 which provides more detailed image quality data for the “contrast” image quality parameter category. The GUI 775 displays the image quality category (i.e., contrast) and optionally includes a list of the possible scores 776 and the selected score 777 similar to what was shown in the image quality summary GUI 720. The GUI 775 also includes a table 778 with more granular image quality detail for a plurality of image quality parameter features related to the “contrast” image quality category. The first column lists the different image quality parameter features, which in this example are low contrast and high contrast. In other embodiments, other image quality parameter features can be shown here, or different combinations of the image quality parameter features currently shown in FIG. 7L may be displayed. The table 778 also includes columns for at least one of the images in the image study that is undergoing VQR with checkboxes (input fields for allowing the reviewer to enter feedback for one or more images of the image study) as was described for GUI 750. The reviewer can click the checkboxes where the image quality parameter features were included in each of the views in these columns. In at least one embodiment, the table 778 may also include columns labelled “Improper KvP” and/or “Uncertain” which the reviewer can check if they think that Improper KvP lead to poor image quality or if they are not certain why there is poor image quality for the image quality parameter features listed along the rows of table 778, for example. In at least one embodiment, the table 778 may also include a column labelled “Other” with text boxes for each of the image quality parameter features. The reviewer can enter text feedback or any other feedback that they have for the image quality parameter features listed in this table 778. The data entered by the reviewer is received by the processor. As with GUI 750, although not shown in FIG. 7L, a “save” input button can be added so that the processor can save all of the entries made by the reviewer to the database 230.

Referring now to FIG. 7M, shown therein is image quality GUI 780 which provides more detailed image quality data for the “sharpness” image quality parameter category. The GUI 780 displays the image quality category (i.e., sharpness) and optionally includes a list of the possible scores 781 and the selected score 782 similar to what was shown in the image quality summary GUI 720. The GUI 780 also includes a table 783 with more granular image quality detail for a plurality of image quality parameter features related to the “sharpness” image quality category. The first column lists the different image quality parameter features, which in this example are poor presentation of linear structures, poor presentation of feature margins and poor presentation of microcalcifications. In other embodiments, other image quality parameter features can be shown here, or different combinations of the image quality parameter features currently shown in FIG. 7M may be displayed. The table 783 includes columns for at least one of the images in the image study that is undergoing VQR with checkboxes (input fields for allowing the reviewer to enter feedback for one or more images of the image study) as was described for GUI 750. The reviewer can click the checkboxes where the image quality parameter features were included in each of the views in these columns. In at least one embodiment, the table 783 may also include columns labelled “Patient Motion” and/or “Uncertain” which the reviewer can check if they think that patient motion leads to poor image quality or if they are not certain why there is poor image quality for the image quality parameter features listed along the rows of table 783, for example. The table 783 also includes a column labelled “Other” with text boxes for each of the image quality parameter features. The reviewer can enter text feedback for any other feedback that they have for the image quality parameter features listed in this table 783. The data entered by the reviewer is received by the processor. As with GUI 750, although not shown in FIG. 7M, a “save” input button can be added so that the processor can save all of the entries made by the reviewer to the database 230.

Referring now to FIG. 7N, shown therein is image quality GUI 785 which provides more detailed image quality data for the “noise” image quality parameter category. The GUI 785 displays the image quality category (i.e., noise) and optionally includes a list of the possible scores 786 and the selected score 787 similar to what was shown in the image quality summary GUI 720. The GUI 785 also includes a table 788 with more granular image quality detail for a plurality of image quality parameter features related to the “noise” image quality category. The first column lists the different image quality parameter features, which in this example are obvious mottle pattern and presentation of detail limited by noise. In other embodiments, other image quality parameter features can be shown here, or different combinations of the image quality parameter features currently shown in FIG. 7N may be displayed. The table 788 includes columns for at least one of the images in the image study that is undergoing VQR with checkboxes (input fields for allowing the reviewer to enter feedback for one or more images of the image study) as was described for GUI 750. The reviewer can click the checkboxes where the image quality parameter features were included in each of the views in these columns. In at least one embodiment, the table 788 may also include a column labelled “Causes” where the reviewer can enter text feedback for any other feedback that they have for the image quality parameter features listed in this table 788. The data entered by the reviewer is received by the processor. As with GUI 750, although not shown in FIG. 7N, a “save” input button can be added so that the processor can save all of the entries made by the reviewer to the database 230.

Referring back to FIG. 7A, once the reviewer is finished with assessing the desired image quality categories that were summarized in GUI 720, the method 700 proceeds to act 710 where the data files 230 are queried to determine if the VQR is an initial VQR or a subsequent VQR that is associated with an active follow-up. If it is not an initial VQR, then the method 700 ends. If it is an initial VQR, the reviewer is asked whether they think the image study (which can also be referred to as a mammogram) is acceptable for interpretation. FIG. 7D provides an example GUI 734 which shows text 736 to pose the question to the reviewer and also has input buttons 738 which allows the reviewer to provide a Yes or No answer to the question by clicking one of the input buttons 738. The reviewer may also provide comments in text input field 740 related to the answer that the reviewer selected. The GUI 734 also provides input buttons 742 and 744 which the reviewer may select in order to initiate a review follow-up or indicate that the review is complete, and no follow-up is needed, respectively. The reviewer's selection or lack of selection of input buttons 742 and 744 as well as other image quality feedback data that is received by the processor from the reviewer may be recorded by the processor in the database 230. In alternative embodiments, determining whether or not a VQR is in an initial or subsequent state may happen at other stages of the method 700.

At act 712, the method 700 runs an algorithm to determine whether or not to recommend that the image study is acceptable for interpretation. The generation of this automated recommendation may be performed by the recommendation module 226 and is based on the image quality data so that the recommendation indicates whether the image study has an overall level of image quality that is acceptable for interpreting the image study to provide an accurate diagnosis. The automated recommendation may then be displayed to the reviewer. For example, one algorithm that can be used in this case to generate the automated recommendation is to sum up the scores across all of the image quality categories, weight the summed scores, total the weighted summed scores and then compare the total weighted summed scores, which may be called a VQR score, to a VQR threshold in order to determining whether initiation of a review follow-up is suitable based on the comparison which indicates whether or not the image study is acceptable for interpretation. The scores may be based on the stored image quality data, or it may be based on scores that were edited by the reviewer.

For example, referring to FIG. 7C, table 730 shows that a value of 1 is provided under the score column for a score that was given to each of the image quality categories and then each of the scores is weighted by row since a different weight may be applied to a different image quality category and then summed (see the row labelled “TOTAL”). Another set of weights (see the row labelled “WEIGHTING”) may then be applied to the summed scores to obtain a subtotal for each score, where scores that indicate poorer quality can be weighted more heavily. In this example, the positioning image quality category may be given a weight of 4 while the other image quality categories are each given a weight of 1/7. The image quality scores may be weighted so that the score A is given a weight of ¼, the score B is given a weight of 0.5, the score C is given a weight of 1 and the score D is given a weight of 2; however, other weights can be used in other embodiments. The subtotals are then added to generate a VQR score, which in this example is 4.43. This value can then be compared to a VQR threshold and if the VQR score is greater than the threshold then the image study is indicated as not being acceptable for interpretation.

In alternative embodiments, the VQR score may be determined differently. For example, other techniques that may be used include, but are not limited to, a weighted score with logistic regression, modeling acceptable vs not acceptable as a function of the 8 image quality categories (i.e., categories A to H scoring) or more parameters. Alternatively, a prognostic index may be used to determine the VQR Score. The prognostic index may be a weighted score derived from regression coefficients. Alternatively, machine learning or classification algorithms that generate a predicted probability score for VQR may be used.

Referring again to FIG. 7A, at act 714, the method 700 determines whether to suggest a follow-up recommendation. This may be determined by comparing the VQR score to a VQR threshold. In this example embodiment, the VQR is 4.43 but it can be different in other embodiments. The VQR threshold can be predefined, but it may also be user adjustable or algorithmically determined in some embodiments.

For example, the VQR threshold may be algorithmically determined (e.g., statistically determined) to find the VQR threshold that best identifies those patients who are likely to have a repeat or recall for further medical imaging to be performed on them due to inadequate image quality from at least one image study that was performed on the patient. For example, assuming a statistical model y=x, y and x data may be collected from patients across one or more departments or institutions to develop, for example, a classification tree (e.g., using a Classification and Regression Tree (CART) algorithm), where the value of y is defined as a technical recall (TR) or no technical recall (No TR) (i.e., the technical recall and no technical recall classes), and x is the computed VQR threshold. The first split of the classification tree provides the cut point on the value for the VQ threshold that optimally separates those patients with ‘TR’ from those with ‘No TR’. A classifier built in this manner, can then be deployed for use in the medical imaging system 100 so that it receives y and x values from new mammograms and generates a TR or No TR classification for that image study. The CART algorithm is just an example of one statistical method that can be used. In other embodiments, logistic regression or other Maximum Likelihood modeling strategies may be used to develop the classifier and the Area Under the ROC curve (AUROC), also known as the C index, may be used to select the optimal point on the ROC curve for the VQR threshold. Examples of using the AUROC to identify the operating point on the ROC curve that is optimal are described in published PCT patent application WO 2020/102914.

The VQR may be defined so that a higher VQR score means that the image quality of the image study is poor based on the scoring system that was selected in this example embodiment. Therefore, if the VQR score is higher than the VQR threshold then the method 700 recommends, via a GUI or pop-up message, that the image study is not acceptable for interpretation (meaning that the image quality of the image study may not be sufficient enough to make a correct diagnosis from reviewing the images of the image study) and there should be a follow-up action. In this case the method 700 proceeds to act 716. Otherwise, if the VQR score is lower than the VQR threshold implying acceptable image quality for the image study then the method 700 ends.

At act 716 of the method 700, the follow-up recommendation and rationale are displayed to the reviewer via a GUI 734 (e.g., see FIG. 7D) or a pop-up message or other electronic notifier including an email and/or text message. However, if it is determined that the follow-up recommendation should be suggested but the reviewer has already selected the answer NO, a modified GUI 734′ (see FIG. 7E) may be displayed in which the reviewer's selection of the input buttons 738 is shown with a highlighted input button 739 and the automated recommendation is shown as text message 742′ above input button 742 that allows the reviewer to change their mind and select the initiation of review follow-up. In this example, the text message 742′ includes the text “MIQA recommends initiating a review follow-up”. Another input button 744 is provided for the reviewer to select if the reviewer still thinks that a review follow-up is not needed.

Alternatively, if the reviewer has not yet selected one of the input buttons 738 then a first pop-up window such as window 745 shown in FIG. 7F can be displayed. The recommendation is shown in area 740 of window 734 and there is an input button 742 that the reviewer can select if they agree with the recommendation. In at least one embodiment, the recommendation rationale may also be displayed to the reviewer using a second pop-up window, such as window 746 in FIG. 7F, in which the recommendation is provided using text 748 and the rationale is shown using text 749. If the reviewer does not agree with the recommendation, then they can select another input button 744 to indicate that the review is complete, and no further action is needed.

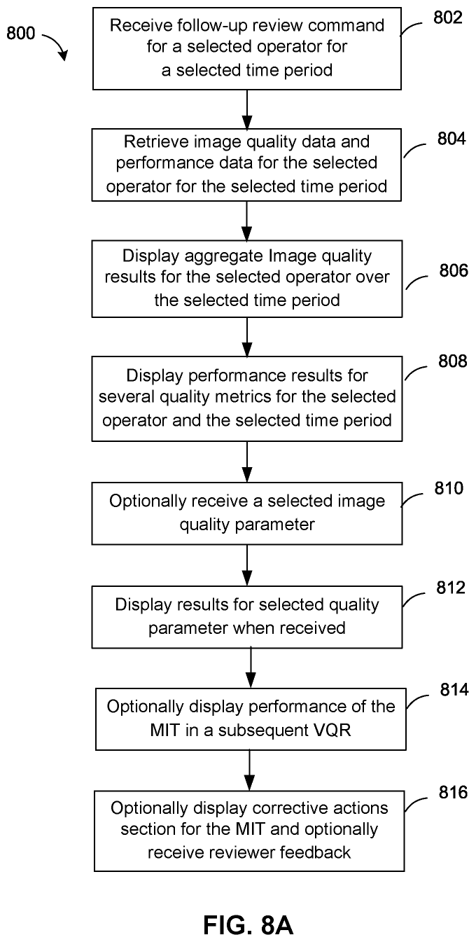

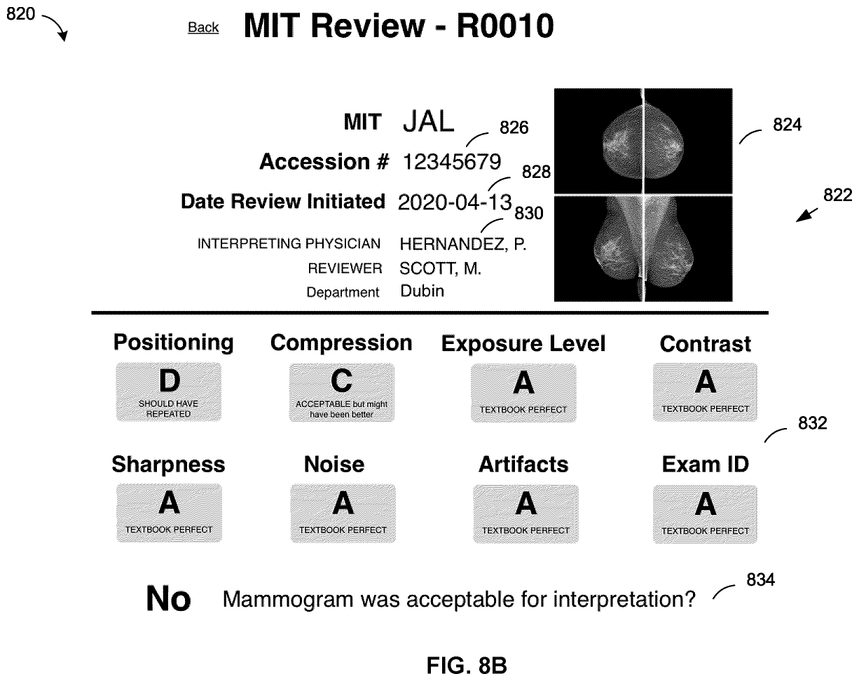

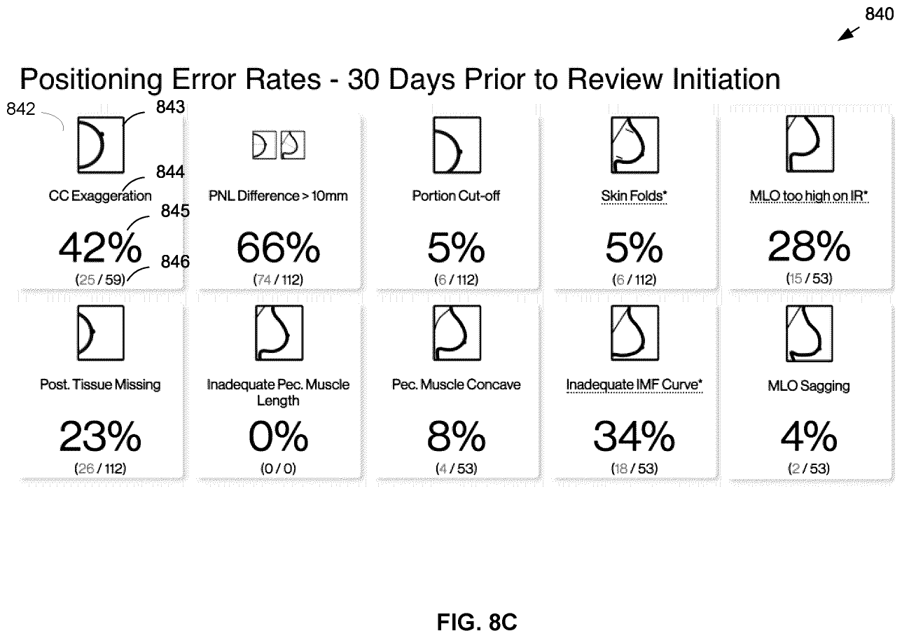

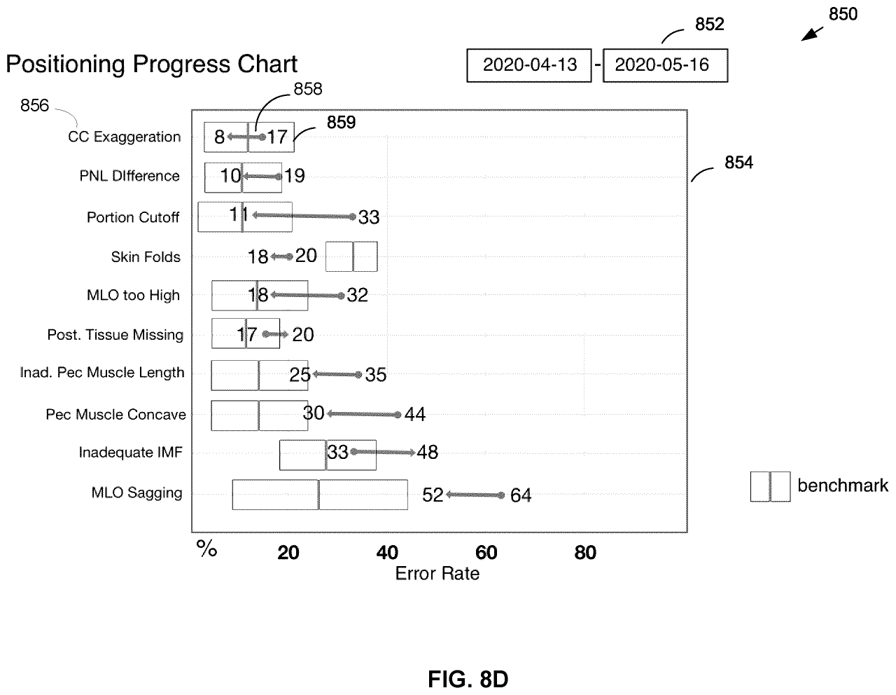

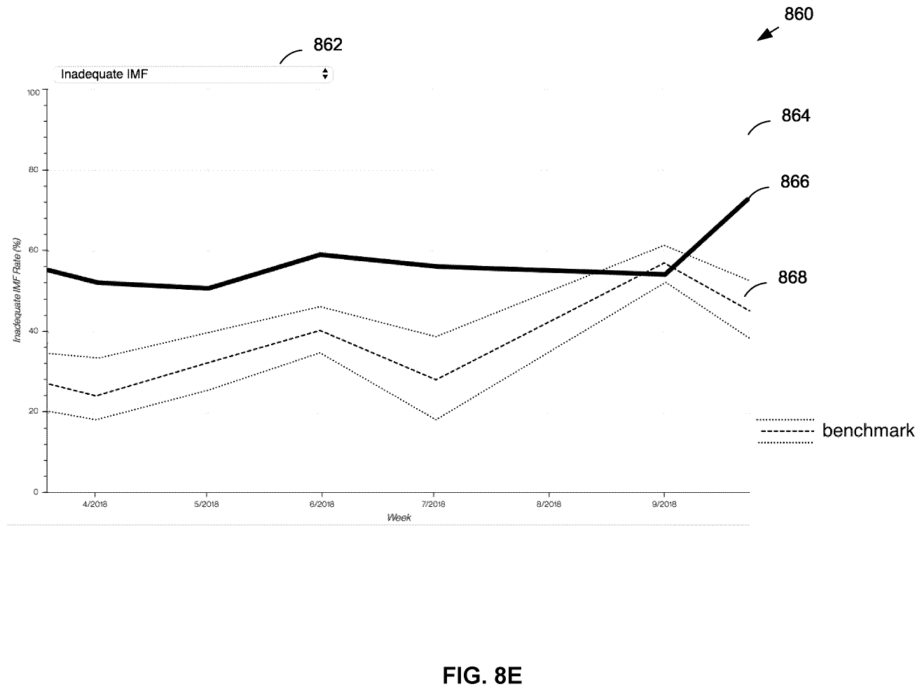

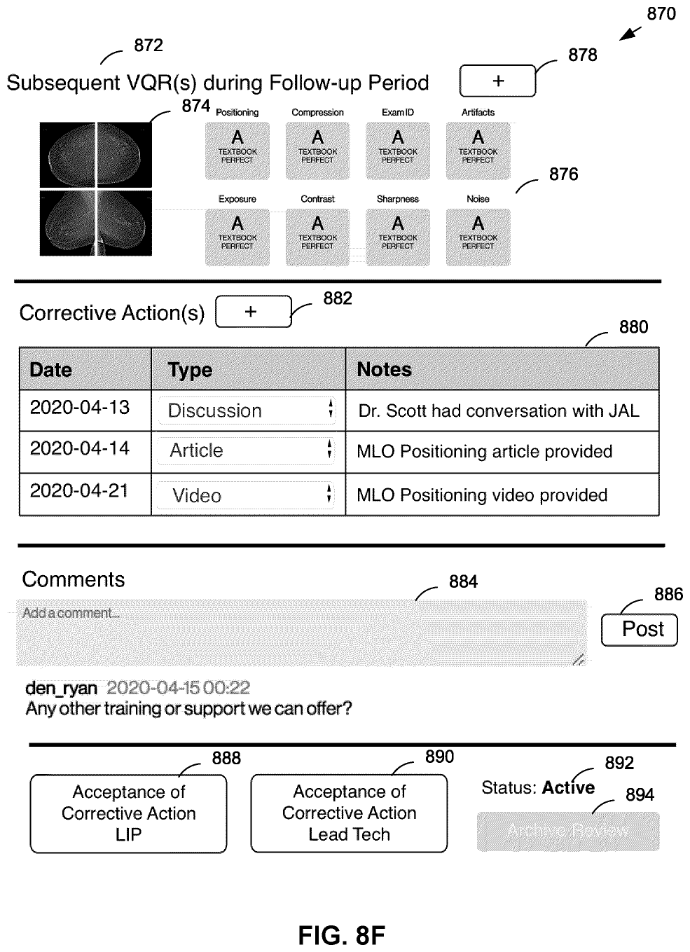

Referring now to FIG. 8A, shown therein is a flow chart for an example embodiment of a method 800 for electronically reviewing image quality of image studies acquired by a Medical Imaging Technologist (MIT) using the medical imaging system 100. The method 800 may include displaying MIT-specific quality performance metrics along with benchmarks, optionally show results from subsequent VQR(s), and optionally show documented corrective actions. Method 800 may be performed when a reviewer decides to perform follow-up on a particular MIT, perhaps after performing a VQR of an image study that was acquired by the MIT. The quality performance metrics may include some image quality parameter features. The benchmarks may be organization-wide or regional such as based on MIT performance for MITs in a given province, a given state or nation-wide.

In at least one embodiment, the method 800 may allow the reviewer to assign corrective action(s) to the MIT.

In at least one embodiment, the method 800 may allow the reviewer to perform subsequent follow-ups on the MIT and link the MIT's performance in a subsequent follow-up to the MIT's performance in a previous follow-up.

In at least one embodiment, the method 800 may display the performance graphically over time to make it easier to assess the MIT's performance for various image quality parameters features.

In at least one embodiment, the method 800 may provide all of the aforementioned functions. Method 800 may be performed by a processor of the processor unit 202 in FIG. 2.

The method 800 begins at act 802 which involves receiving an electronic request, such as a follow-up review command from a reviewer to perform a review on a selected operator (i.e., MIT) for a selected time period is received. At act 804, the method 800 retrieves image quality data that corresponds to the image quality data for at least one image study performed by the MIT and retrieving performance data for the MIT for the selected time period from at least one database, such as the database 230. The performance data is based on errors identified in the image quality data of the images reviewed by the MIT, which can be determined by the MIQA server 200 (e.g., via the image quality analysis module 222 by implementing techniques described in PCT application publication WO 2020/102914).

At act 806, the method 800 includes generating an MIT review GUI that includes at least a portion of the image quality data and the MIT performance data. At least some of the image quality data and the performance data for the selected MIT over the selected time period are then displayed on the MIT review GUI, such as GUI 820. Portions of the GUI 820 are shown as GUI portions 822, 840, 850, 860 and 870 in FIGS. 8B to 8F, respectively. Alternatively, in another embodiment, the GUI portions 822, 840, 850, 860 and 870 may be displayed as separate GUI windows. For ease of illustration, the description will refer to GUI portions 822, 840, 850, 860 and 870.

Referring again to act 806, as an example, the initial VQR results as well as the aggregate image quality results for the selected MIT over the selected time period may be displayed. For example, a first GUI portion 822 of GUI 820 in FIG. 8B includes an image region 824 where images are shown of the image study reviewed during the initial VQR review for a particular MIT. The GUI portion 822 may display one or more of an identifier 826 for identifying the MIT including a code “JAL” and an accession number, a date identifier 828 for the date on which the review was initiated, and an identifier 830 indicating the IP who interpreted the image study, the reviewer and the department of the institution where the image study was acquired. The GUI portion 822 may also have an image quality summary section 832 that provides VQR results for one or more image quality categories for the initial VQR of the image study. In this example, the image quality categories are positioning, compression, exposure level, contrast, sharpness, noise, artifacts and exam ID and the scores can be shown as various values like A, B, C or D as was previously described for FIGS. 7A-7N. In other embodiments other image quality categories may be displayed or a different combination of the image quality categories show in FIG. 8B may be displayed. The GUI portion 822 may optionally also include text message 834 which displays an assessment of whether the image study was acceptable for interpretation, which in this example is negative, since a positioning score of “D” alone is enough to do a review in most cases.

Referring back again to FIG. 8A, the method 800 then proceeds to act 808 where the performance results for the selected MIT over the selected time period are displayed for one of the image quality categories shown in GUI portion 822. An example of this is provided by GUI portions 840 and 850 shown in FIGS. 8C and 8D. The performance results shown in GUI portions 840 and 850 will change based on which one of the image quality categories of GUI portion 821 are selected. In this example, the reviewer selected the positioning image quality category.

Referring now to FIG. 8C, in GUI portion 840, one or more image quality parameters are displayed for the selected image quality category (i.e., the positioning error category) for a selected time period, such as 30 days (or another time period), prior to the initiation of the review. Using the “CC exaggeration” image quality parameter as an example, the GUI 840 provides subwindows 842 to display data about the performance of the MIT for the different image quality parameters that are used in the selected image quality category, a graphical representation 843 to explain what the image quality parameter represents in an image, a text field 844 to display the name of the image quality parameter, a percentage indicator 845 to indicate the percentage of images of all of the images that were acquired by the MIT during the selected time period that satisfy a particular operating point (i.e. are unacceptable) for the image quality parameter, and an identifier 846 showing the number of images that were unacceptable for this image quality parameter over the total number of images that were assessed. The particular operating point is set when the image quality data is generated as is explained in PCT application publication WO 2020/102914. In other embodiments, a given subwindow 842 may not show each of elements 843 to 846. The benefit of showing the image quality parameters for the MIT in FIG. 8C is that a reviewer can quickly see issues that the MIT may be having in acquiring medical images based on which image quality parameters are problematic for the MIT. For example, a glance view of the image quality aspects shown in the GUI portion 840 may indicate one or more aspects of the positioning error that have the highest incidence rate (e.g., shown by reference number 845) and these can be referred to as the ‘top errors’. Count of images assessed within the time period is part of the visualization (as shown by reference number 846). For example, the data highlighted by reference numeral 846 allows the viewer can easily observe the number of images assessed to compute the incidence rate of 845. This may be important to know, because a high incidence rate is not meaningful if the number of images assessed was very low. (e.g., if a MIT only worked for a few days in the period of evaluation, any statistics for a small number of image studies would not be meaningful). If the number of images assessed is suitably large (e.g., >50), then the incidence rate becomes more meaningful.

Referring now to FIG. 8D, in GUI portion 850, a progress chart 854 for one or more image quality parameters for a selected time period, that the reviewer may select via input fields 852, is displayed to statistically show a change in MIT performance for at least one of the image quality parameters, such as positioning errors, for example. This may be done for all of the various image quality parameters of FIG. 8C to show how they vary during the selected time period for the MIT. The change is displayed with an extent to visually show if there is a big or small change in MIT performance and directionality to show if there is an improvement or worsening of the MIT performance. Other statistical metrics of variation other than the minimum/maximum for the selected time period can be shown in other embodiments. For example, the standard deviation can be computed and 2*sigma of the daily or weekly error rates over a particular period can be displayed in a chart. Again using the CC Exaggeration image quality parameter 856 as an example, an identifier 858 is displayed to show how the MIT has been operating with upper and lower numbers showing the start and end points of the error over the time period for the MIT and an arrow showing that the error is decreasing (i.e., MIT performance is improving) when the arrow points to the left or that the error is increasing (i.e., MIT performance is getting worse) when the arrow points to the right. The arrows can also be given different colors to quickly visually indicate that the error is improving (e.g., the arrow can have the color blue or green) or that the error is getting worse (e.g., the arrow can have the color yellow or red). The progress chart 854 allows the reviewer to also quickly determine which positioning errors are more problematic for the MIT based on which positioning errors are closer to the right side of the progress chart 854.

In at least one embodiment, a benchmark indicator 859 may also be displayed using a rectangle with an interior vertical line showing the mean and the left and right sides of the rectangle showing the upper and lower confidence levels for other MITs. This allows the reviewer to more quickly visualize the performance of the MIT versus the benchmark that is based on other MITs. The benchmark may be determined at the clinic, hospital, regional (i.e., provincial or state) health system or at the national level. In an alternative embodiment, the reviewer can override the defaults to set user-defined values for the mean and confidence limits. For example, the reviewer may set values for one or more benchmarks based on a panel of experts, a delphi panel discussion or a quality committee determining what values would be acceptable for the one or more benchmarks.

Referring back again to FIG. 8A, the method 800 then proceeds to act 810 where the method 800 involves receiving a selected image quality parameter, and act 812 where the method 800 involves generating a second performance graph for the MIT performance for the selected image quality parameter; displaying the MIT performance for the selected image quality parameter and displaying performance benchmark data for the selected image quality parameter in the MIT review GUI to the reviewer. An example of this is GUI portion 860 shown in FIG. 8E which displays the error rate for a selected image quality parameter over the selected time period. The GUI portion 860 has a drop-down menu 862 from which the reviewer can select the image quality parameter for which the MIT performance is shown in the graph 864. In this example, the reviewer selected the inadequate IMF image quality parameter, which is then displayed on the label of the y-axis of the graph 864. The solid line 866 in the graph 864 shows the actual performance of the MIT for this image quality parameter while the dashed lines show how the benchmark 868 for this image quality parameter varies over the selected time period. The benchmark 868 is shown with the middle dashed line indicating the mean and the upper and lower dashed lines showing the upper and lower confidence limits. The benchmark 868 can be computed from the institutional data or from a combination of data across institutions locally, regionally or nationally. The GUI 860 enables the reviewer to see how the performance of the MIT is trending across time and whether the performance is getting better or worse for a selected image quality parameter and how the MIT's performance compares to the benchmark over time.

Referring back to FIG. 8A, the method 800 can optionally include act 814 for generating and displaying another GUI portion displaying performance of the MIT for a subsequent VQR in the MIT review GUI, an example of which is shown as GUI portion 870 in FIG. 8F. The GUI portion 870 includes a first region 872 that shows the performance of the MIT in the subsequent VQR. Images 874 for the image study that was reviewed for the subsequent VQR may be displayed along with image quality data 876 showing scores for different image quality categories for the VQR. The GUI 820 may also include an “add” button 878 which is a subsequent review input option that the reviewer may select in case the reviewer wants to add another subsequent VQR for the review of the MIT. The GUI 820 may be saved as a report for the performance of the MIT for a given time period.

Referring back to FIG. 8A, in at least one embodiment, the method 800 may also include act 816 where the GUI portion 870 is displayed to include an optional corrective actions section with a table 880 showing the corrective actions that have been recommended to the MIT and additional data on whether the recommended corrective actions were taken for the MIT. The first column of table 880 indicates the date of the corrective action, the second column indicates the type of corrective action (e.g., a discussion, article, video, course, etc.) and the third column indicates notes to provide further details regarding the corrective action. The GUI 820 includes a corrective action input option (e.g., the “add” button 882) to allow the review to add input details for at least one new corrective action for the MIT to perform the reviewer thinks that performing these one or more new corrective actions will help the image quality performance of the MIT to improve. The reviewer can select the “add” button 882 a desired number of times which will allow the reviewer to add a corresponding desired number of rows to the table 880 and input details for the new corrective action(s) in these new rows. Any added new corrective actions are then saved.

In at least one embodiment, the GUI portion 870 may also include a comment text box 884 to allow the reviewer to add comments related to the progress or challenges of the MIT, or comments related to how any of the corrective actions were received by the MIT (e.g., what the MIT's thoughts were for performing the corrective actions) and/or how the corrective actions were performed by the MIT, or any other comments and post them to the follow-up review or save this feedback data when the reviewer selects the button 886.

In at least one embodiment, a “review report” GUI may be accessible by the MIT, whose performance was just assessed, where the review report GUI shows the table 880 and the comment box 884. The MIT's interaction with this GUI can be tracked by the MIQA server 200 in terms of recording the MIT's actions for completing the recommended corrective actions. For example, a review flag can be set that confirms that the MIT has viewed the review report GUI. In at least one embodiment, the amount of time (i.e., MIT review time) that the MIT spent reviewing the review report GUI can also be recorded. The review flag and optionally the MIT review time can be recorded in the database 230 and then shown in the GUI 820 during a subsequent review. In at least one embodiment, the review report GUI may also include links (e.g., hyperlinks) to facilitate the MIT performing the recommended corrective action. For example, the link may be to a video, an article, other electronic material or to send an electronic message to another more experienced MIT to setup a meeting to receive mentoring. The actions of the MIT in terms of selecting the link and taking the recommended corrective action can also be recorded in the database 230 and then shown in the GUI 820 during a subsequent review. This allows the activity of the MIT in reviewing their review report and taking any recommended corrective actions to be digitized and tracked, which ensures that patterns for any review and performance of remedial actions by an MIT are recorded so that it is readily known which MITs have engaged in the assigned recommended corrective actions.

In at least one embodiment, the MIQA server 200 may automatically track and report on the activities completed by the MIT, for activities related to the recommended corrective actions. This may include performing reviews of additional studies that are accessed by the MIQA server 200, (e.g., identified by the reviewer as relevant to the issue at hand), reading or monitoring related instructional material available directly to (or through links) the MIQA server 200, or self-reviewing all newly acquired studies by the MIT for some remedial period.

In at least one embodiment, the GUI portion 870 may also include input buttons 888 and/or 890 that may be selected by the reviewer to finalize the follow-up review. The LIP can select the input button 888 once they have agreed with the performance improvement of the MIT based on the applied corrective action(s) 880 and the image quality performance data shown in GUI portions 840, 850 and 860. The Lead tech or QC Manager can select the input button 890 once they have agreed with the performance improvement of the MIT based on the applied corrective action(s) 880 and the image quality performance data shown in GUI portions 840, 850, and 860.

In at least one embodiment, the GUI portion 870 may also include a status identifier 892 to indicate whether the review of the MIT is still active, has been completed, or is archived. Until the LIP and QC Manager select the input buttons 888 and 890 the status is active. Once the LIP and QC Manager select the input buttons 888 and 890 the status is changed to complete. After a period of time the reviewer may decide to hide older reviews, in which case then they can select the input button 894 to archive the review. The data related to the review is then stored by the MIQA application 220 in the database 230.

It should be noted that in at least one embodiment, one or more of the GUIs described herein are output on a display device. Alternatively, in at least one embodiment, one or more of the GUIs described herein are output on a printed report and/or stored on a storage device. Alternatively, in at least one embodiment, one or more of the GUIs described herein are output on a display device, a printed report and/or stored on a storage device.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

### REFERENCES

Abdolell M, Payne J, Caines J, Tsuruda K, Barnes P, Talbot P, Tong O, Brown P, Rivers-Bowerman M, Iles S (2020). Assessing breast cancer risk within the general screening population. Developing a breast cancer risk model to identify higher risk women at mammographic screening. Eur Radiol. 2020 May. https://doi.org/10.1007/s00330-020-06901-x
Ekpo EU., et al. (2014). Optimisation of direct digital chest radiography using Cu filtration. Radiography, 20, pp. 346-50.
Kang, B. S., Park, S. C., Decision Support Systems 29 (2000) 59-72.
Kourti, T., Macgregor, J. F., Multivariate SPC methods for process and product monitoring, J. Qual. Technol. 28 (4). (1996). 409-428.
Guertin, M H., et al. (2018) Mammography Clinical Image Quality and the False Positive Rate in a Canadian Breast Cancer Screening Program. Canadian Association of Radiologists Journal, Volume 69, Issue 2, pp. 169-175.
Mason, R. L., Champ, C. W., Tracy, N. D., Wierda, S. J., Young, J. C., Assessment of multivariate process control techniques, J. Qual. Technol. 29 (2). (1997). 140-143.
Montgomery, D. C., 2005. Introduction to statistical quality control. Hoboken, N. J.: John Wiley.
Oakland, J. S., 2007. Statistical process control. Amsterdam: Elsevier Butterworth-Heinemann.
O'Leary, D. and Rainford, L. (2011). Can radiation dose in mammography be further reduced by increasing the image quality?. Breast Cancer Research, 13(Suppl. 1), pp. S1-S7.
Rauscher, G., et al. (2013). Mammogram image quality as a potential contributor to disparities in breast cancer stage at diagnosis: an observational study. BMC Cancer, 13(208), published 2013 Apr. 26. doi:10.1186/1471-2407-13-208.
Taplin, S., et al. (2002). Screening Mammography: Clinical Image Quality and the Risk of Interval Breast Cancer. American Journal of Roentgenology, 178(4), pp. 797-803.
Teyarachakul S. et al., European Journal of Operational Research 178 (2007) 472-481.
Yala A, Lehman C, Schuster T, Portnoi T, Barzilay R. A Deep Learning Mammography-based Model for Improved Breast Cancer Risk Prediction. Radiology. 2019; 292(1):60-66. doi:10.1148/radio1.2019182716

1. A computer-implemented method for performing image quality review of medical images, wherein the method is performed by a processor and the method comprises:
receiving an indication that an image study is being retrieved for viewing at a computer device by a user and an image study ID for the image study;
retrieving image quality data that corresponds to the image study based on the image study ID, the image quality data being retrieved from a database;
generating an image quality Graphical User Interface (GUI); and
displaying the image quality GUI along with at least some of the image quality data at the computing device.

2. The method of claim 1, wherein the image quality GUI that is displayed comprises a window that includes an image quality metric that summarizes the image quality of the image study.

3. The method of claim 2, wherein the method further comprises displaying additional measures in the image quality GUI where the additional measures are related to one or more images of the image study and comprise: breast density data, cancer risk data, a priority score or any combination thereof.

4. The method of claim 2 or claim 3, wherein the image quality GUI is generated and displayed when the image quality metric indicates the image study is inadequate for making a correct diagnosis by comparing the image quality criterion an image quality criterion.

5. The method of claim 4, wherein the method further includes presenting the user with a list of follow-up tasks including any combination of: (a) displaying an enhanced view of the image quality data for the image study; (b) scheduling a follow-up visual quality review of the image study; (c) sending an electronic message with the image study ID for electronic documentation and creation of a report of a review of the image study; (d) sending an electronic notification message to prioritize the image study for review; (e) sending an electronic notification message to perform a follow-up action on a patient from whom the image study was obtained; and (f) sending an electronic request message to another user to review the image study to provide a second assessment.

6. The method of claim 1, wherein the image quality GUI that is displayed comprises a subwindow having a plurality of image quality data for different images of the image study.

7. The method of claim 6, wherein the subwindow further includes images of the image study.

8. The method of claim 6 or claim 7, wherein the image quality data shown in the subwindow comprises names of image parameter feature scores and scores or image quality symbols for the image parameter feature scores.

9. The method of claim 8, wherein the image quality symbols comprise error symbols or pass symbols and when the user selects an edit function in the image quality GUI the method further includes displaying an opposite image quality symbol for any image quality symbols selected by the user.

10. The method of any one of claims 6 to 9, wherein the method comprises displaying an input button in the image quality GUI for allowing the user to select that the image study is to be sent for Visual Quality Review (VQR) and the method comprises flagging the image study for VQR upon receipt of the input button being selected by the user.

11. The method of any one of claims 6 to 9, wherein the method comprises generating a Visual Quality Review (VQR) recommendation and displaying the VQR recommendation at the computing device.

12. The method of claim 11, wherein the method comprises generating the VQR recommendation by comparing the image quality metric to an image quality threshold.

13. The method of claim 11 or claim 12, wherein upon receiving a command from the user to send the image study to VQR, the method comprises electronically documenting that the image study is to be sent for VQR.

14. The method of claim 11 or claim 12, wherein upon receiving a command from the user to send the image study to VQR, the method comprises updating the image quality GUI to display that the image study is to be sent for VQR.

15. The method of any one of claims 1 and 6 to 14, wherein the method further comprises displaying a subwindow that includes breast density data in the image quality GUI.

16. The method of any one of claims 1 and 6 to 15, wherein the method further comprises displaying a another subwindow that includes cancer risk data in the image quality GUI.

17. The method of any one of claims 1 and 6 to 16, wherein the method further comprises displaying a an additional subwindow that includes priority score data in the image quality GUI.

18. The method of claim 3, wherein the method further comprises generating the priority score using the image quality data, the breast density data and the cancer risk data.

19. The method of claim 18, wherein the priority score is generated using a decision tree having a first level where the cancer risk data is stratified between a standard risk score and a priority risk score based on comparing a priority score value to a priority score threshold, a second level where the breast density data is stratified between high density or low density based on comparing a breast density value to a breast density threshold and a third level where the image quality data is stratified between high quality and poor quality based on comparing an overall image quality value for the image study to an image quality threshold.

20. An electronic device for providing image quality review of medical images in a medical imaging system, wherein the electronic device comprises:
a memory unit that includes software instructions for visualizing image quality data;
a network unit for communicating with other devices and software programs in the medical imaging system; and
a processor unit in communication with the memory unit and the network unit, the processor unit having at least one processor that is configured to:
receive an indication that an image study is being retrieved for viewing at a computing device by a user and an image study ID for the image study where the computing device is electronically connected to the medical imaging system;
retrieve image quality data that corresponds to the image study based on the image study ID, the image quality data being retrieved from a database;
generate an image quality Graphical User Interface (GUI); and
display the image quality GUI along with at least some of the image quality data at the computing device.

21. The electronic device of claim 11, wherein the at least one processor unit is further configured to perform the method as defined in any one of claims 2 to 19.

22. A computer readable medium comprising software instructions, which when executed by an electronic device, configure the electronic device to perform the method according to any one of claims 1 to 19.

23. A computer-implemented method for automatically identifying image studies for image quality review using a medical imaging system, wherein the method is performed by a processor and the method comprises:
receiving an indication that a new image study has been acquired and an image study ID for the new image study;
obtaining image quality data for images in the new image study;
determining when the image quality data meets Visual Quality Review (VQR) criteria; and
updating a VQR worklist file to include the image study ID when the image quality data meets the VQR criteria.

24. The method of claim 23, wherein the method comprises:
generating and displaying an image quality search Graphical User Interface (GUI) that provides input fields to allow a user to enter the VQR criteria;
receiving the VQR criteria from the user; and
saving the received VQR criteria.

25. The method of claim 24, wherein the method comprises displaying an input option for allowing the user to select one or more of views or a portion of the images in the new image study the VQR criteria are applied to.

26. The method of claim 24 or claim 25, wherein the method comprises (a) displaying input options to the user to allow the user to specify the VQR criteria that are applied to certain images of the new image study, (b) receiving at least one VQR criterion from the user or a user defined combination, via at least one logical operator, of at least two VQR criteria where each VQR criteria, where each VQR criteria involves an image parameter feature that is selected by the user, a comparison operator that is selected by the user and a threshold value that is selected by the user; and (c) storing these user selections for the VQR criteria.

27. The method of any one of claims 23 to 26, wherein the method comprises keeping track when a number of image studies that were acquired by a given Medical Imaging Technologist (MIT) meet the VQR criteria over a certain time period, VQR review for the given MIT is electronically noted.

28. The method of any one of claims 23 to 26, wherein the method comprises keeping track when a number of image studies that were acquired by a given MIT are flagged based on the VQR criteria and determining whether an overall image study quality, across all of the flagged image studies, for the MIT drops below a typical image quality level for the MIT over a certain time period.

29. An electronic device for automatically identifying image studies for image quality review in a medical imaging system, wherein the electronic device comprises:
a memory unit that includes software instructions for visualizing image quality data;
a network unit for communicating with other devices and software programs in the medical imaging system; and
a processor unit in communication with the memory unit and the network unit, the processor unit having at least one processor that is configured to perform the method according to any one of claims 23 to 28.

30. A computer readable medium comprising software instructions, which when executed by an electronic device, configure the electronic device to perform the method according to any one of claims 23 to 28.

31. A method for randomly generating a list of image studies for Visual Quality Review (VQR) using a medical imaging system, wherein the method is performed by a processor and the method comprises:
displaying a first section for a random search Graphical User Interface (GUI), where the first section includes at least one first input option to allow a user to select one or more initial search criteria;
displaying a second section for the random search GUI where the second section includes at least one second input option for allowing the user to select one or more stratifying factors;
displaying a third section for the random search GUI where the third section includes a third input option to allow the user to specify a desired number of images studies for VQR for a given pairing of a Medical Imaging Technologist (MIT) and an Interpreting Physician (IP) in a random sample where the MIT is a person who acquires the image studies and the IP is a person who reviews image quality of the image studies;
receiving the user selections for the input options;
generating a random list of image studies for VQR using the user selections; and
storing the random list of the image studies for VQR.

32. The method of claim 31, wherein the method further comprises displaying a recommended number of image studies for VQR for the given pairing of the MIT and the IP in the random sample.

33. The method of claim 32, wherein the method further comprises displaying a number of image studies for VQR based on the user selections.

34. The method of any one of claims 30 to 32, wherein the at least one first input option includes an institution, a department for the institution, a date range, one or more MIT selections and/or one or more IP selections.

35. The method of claim 34, wherein the method further comprises displaying a potential number of image studies for VQR based on the user selections to the at least one first input option.

36. The method of any one of claims 30 to 33, wherein the one or more stratifying factors include scanner model, breast density value and/or image quality metric score.

37. An electronic device for randomly generating a list of image studies for Visual Quality Review (VQR) using a medical imaging system, wherein the electronic device comprises:
a memory unit that includes software instructions for visualizing image quality data;
a network unit for communicating with other devices and software programs in the medical imaging system; and
a processor unit in communication with the memory unit and the network unit, the processor unit having at least one processor that is configured to perform the method according to any one of claims 31 to 36.

38. A computer readable medium comprising software instructions, which when executed by an electronic device, configure the electronic device to perform the method according to any one of claims 31 to 36.

39. A method for electronically performing Visual Quality Review (VQR) on at least one image study that is acquired by a Medical Imaging Technologist (MIT) using a medical imaging system, wherein the method is performed by a processor and the method comprises:
sending an electronic request to a reviewer to perform VQR on a first image study;
retrieving image quality data that corresponds to the image study based on the image study ID, the image quality data being retrieved from a database;
generating one or more image quality Graphical User Interfaces (GUIs) that include at least a portion of the image quality data;
displaying the one or more image quality GUIs; and
receiving and storing image quality feedback data from the reviewer.

40. The method of claim 39, wherein the one or more GUIs include a summary of the image quality data including image quality categories, an index of possible scores for the image quality categories and a score value for the image quality categories.

41. The method of claim 39 or claim 40, wherein the one or more GUIs include an image quality category; optionally a list of possible scores for the image quality category and a score value the image quality category; and a list of image quality parameter features for the image quality category.

42. The method of claim 41, wherein the one or more GUIs include input fields for the list of image quality parameter features of the image quality category for one or more images of the first image study.

43. The method of any one of claims 40 to 42, wherein the image quality categories comprise positioning, compression, exam ID, artifacts, exposure, contrast, sharpness, noise or any combination thereof.

44. The method of any one of claims 39 to 43, wherein the method further comprises generating an additional GUI to allow the reviewer to select whether to initiate a review follow-up or to indicate that the VQR is complete, and no further action is needed; and receiving a selection from the reviewer.

45. The method of any one of claims 39 to 44, wherein the method further comprises generating an automated recommendation on whether the first image study had an overall level of image quality that is acceptable for interpreting the image study to provide an accurate diagnosis and displaying the automated recommendation to the reviewer.

46. The method of claim 45, wherein the method further comprises generating the automated recommendation by generating a VQR score, comparing the VQR score to a VQR threshold; and determining whether initiation of a review follow-up is suitable based on the comparison.

47. The method of claim 46, wherein the VQR score is generated based on a weighted sum of image parameter feature scores across image quality categories from the image quality data for the first image study.

48. The method of claim 46 or claim 47, wherein the VQR threshold is a predefined value, a prognostic index based on a weighted score from regression coefficients or is determined using an algorithm where the VQR threshold is selected to identify patients who are likely to have a recall for further medical imaging to be performed on them due to inadequate image quality from at least one image study performed on the patient.

49. The method of claim 48, wherein the algorithm involves applying a statistical model to patient data to generate a classifier that employs a technical recall and no technical recall classes, where the statistical model uses a classification tree, logistic regression or Maximum Likelihood.

50. An electronic device for electronically performing Visual Quality Review (VQR) on at least one image study that is acquired by a Medical Imaging Technologist (MIT) using a medical imaging system, wherein the electronic device comprises:
a memory unit that includes software instructions for visualizing image quality data;
a network unit for communicating with other devices and software programs in the medical imaging system; and
a processor unit in communication with the memory unit and the network unit, the processor unit having at least one processor that is configured to perform the method according to any one of claims 39 to 49.

51. A computer readable medium comprising software instructions, which when executed by an electronic device, configure the electronic device to perform the method according to any one of claims 39 to 49.

52. A method for electronically reviewing image quality of image studies acquired by a Medical Imaging Technologist (MIT) using a medical imaging system, wherein the method is performed by a processor and the method comprises:
receiving an electronic request from a reviewer to perform a review on the MIT for a selected time period;
retrieving image quality data that corresponds to image quality data for at least one image study performed by the MIT and retrieving MIT performance data for the MIT for the selected time period, the image quality data and performance data being retrieved from at least one database;
generating a MIT review Graphical User Interface (GUI) that includes at least a portion of the image quality data and the MIT performance data; and
displaying the MIT review GUI on a computing device used by the reviewer.

53. The method of claim 52, wherein the MIT review GUI includes images from an image study that the MIT performance is being reviewed for, and an image quality summary section that includes Visual Quality Review (VQR) results for one or more image quality categories.

54. The method of claim 53, wherein the MIT review GUI includes an assessment of whether the image study was acceptable for interpretation.

55. The method of any one of claims 52 to 54, wherein the MIT review GUI further includes one or more image quality parameters for a selected image quality category for a selected time period.

56. The method of claim 55, wherein for a given image quality parameter, a text field is shown to display a name of the image quality parameter, a percentage indicator is shown to indicate a percentage of all of images that were acquired by the MIT during the selected time period that satisfy a particular operating point for the image quality parameter, and an identifier is shown to indicate a number of images that were unacceptable for the image quality parameter over the total number of images that were assessed.

57. The method of any one of claims 52 to 56, the MIT review GUI includes a progress chart for one or more image quality parameters to show a change in MIT performance for the one or more image quality parameters.

58. The method of claim 57, wherein the change in MIT performance is displayed with an extent to visually show if there is a big or small change in the MIT performance and a directionality to show if there is an improvement or worsening of the MIT performance.

59. The method of any one of claims 52 to 58, wherein the method further comprises receiving a selected image quality parameter; generating a second performance graph for the MIT performance for the selected image quality parameter; displaying the MIT performance for the selected image quality parameter and displaying performance benchmark data for the selected image quality parameter in the MIT review GUI.

60. The method of any one of claims 52 to 59, wherein the method further comprises displaying performance of the MIT for a subsequent VQR in the MIT review GUI by showing images from another image study that was reviewed for the subsequent VQR along with includes the VQR results for one or more image quality categories for the subsequent VQR.

61. The method of claim 60, wherein the MIT review GUI includes a subsequent review input option to allow the reviewer to add another subsequent VQR for review of the MIT.

62. The method of any one of claims 52 to 61, wherein the method further comprises displaying a corrective actions section showing corrective actions that have been recommended to the MIT and additional data on whether the recommended corrective actions were taken for the MIT.

63. The method of claim 62, wherein the method comprises providing a corrective action input option to allow the reviewer to add input details for at least one new corrective action for the MIT to perform, and saving any added new corrective actions.

64. The method of claim 62 or 63, wherein the method comprises providing comment text box to allow the reviewer to add comments related to progress or challenges of the MIT; or comments related to how any of the corrective actions were received and/or performed by the MIT; and saving any comments entered by the reviewer.

65. The method of any one of claims 62 to 64, wherein the method comprises generating a review report GUI that is accessible by the MIT to provide the MIT with any recommended corrective actions to improve image quality performance; recording interaction of the MIT with the review report GUI and recording behaviour by the MIT in performing any of the recommended corrective actions.

66. An electronic device for electronically reviewing image quality of image studies acquired by a Medical Imaging Technologist (MIT) using a medical imaging system, wherein the electronic device comprises:
a memory unit that includes software instructions for visualizing image quality data;
a network unit for communicating with other devices and software programs in the medical imaging system; and
a processor unit in communication with the memory unit and the network unit, the processor unit having at least one processor that is configured to perform the method according to any one of claims 52 to 65.

67. A computer readable medium comprising software instructions, which when executed by an electronic device, configure the electronic device to perform the method according to any one of claims 52 to 65.
